SEQUENCE LISTING (135 sequences)

SEQ ID NO: 1 - DNA, 118 bp, Homo sapiens
agagctcattcccgagtttccaagaatttgctgaattgcttttctctgttatctgcttctttgggttatcttgacgaatcagattacaacccatctgagtggagccctggcttctcag

SEQ ID NO: 2 - DNA, 405 bp, Homo sapiens
attcccgagtttccaagaatttgctgaattgcttttctctgttatctgcttctttggactagctctgtgcatcacagagtggggcatggtgttccacatgggaaactgttaaaacagaaatcagaggagccatcggtgtcaatacccttcctacaaactgcattattaagaggttcagggagtcttgggcacagaccaagccaggagatggataaaatgttaaaaaatcaagcaacttctgctacttctgaaaaggataatgatgatgaccaaagtgacaagggtacttataccattgagttagagaatcccaacagtgaggaagtggaagcaagaaaaatgattcacaaggttatcttgacgaatcagattacaacccatctgagtggagccctggcttctcag

SEQ ID NO: 3 - DNA, 370 bp, Homo sapiens
cacacaattattggttatgactatttatcacctaagaactaaacagtggtgctttgatttgagtttgtttactcgagaggcagagtatgttttatctcaatgaatgagagttcctgttgcttcacatccttgccagcatttggcgatgtcattgctttggattttagtcattctgatacagatgttccccaacttacaatggtgaagaagtagaactcagagaagttttacttgccaaaagttctgcagcaagtcagtggcaaattcaaataagtgtacactcatgtatacttagctcagtgtttaactcatggaggttatcttgacgaatcagattacaacccatctgagtggagccctggcttctcag

SEQ ID NO: 4 - DNA, 152 bp, Homo sapiens
gtctaagccgatcatttcattaatattaaactccagagctcattcccgagtttccaagaatttgctgaattgcttttctctgttatctgcttctttgggttatcttgacgaatcagattacaacccatctgagtggagccctggcttctcag

SEQ ID NO: 5 - DNA, 394 bp, Homo sapiens
tttgatttgagtttgtttactcgagaggcagagtatgttttatctactagctctgtgcatcacagagtggggcatggtgttccacatgggaaactgttaaaacagaaatcagaggagccatcggtgtcaatacccttcctacaaactgcattattaagaggttcggggagtcttgggcacagaccaagccaggagatggataaaatgttaaaaaaatcaagcaacttctgctacttctgaaaaggataatgatgatgaccaaagtgacaagggtacttataccattgagttagagaatcccaacagtgaggaagtggaagcaagaaaaatgattcacaaggttatcttgacgaatcagattacaacccatctgagtggagccctggcttctcag

SEQ ID NO: 6 - DNA, 453 bp, Homo sapiens
agcactaaggagagttgtaaggagttgcttttggtccttgctgggaaatgctctgaaaactgaaccagctactgaacacaaaggctgacggtgttgtccctcctgactagctctgtgcatcacagagtggggcatggtgttccacatgggaaactgttaaaacagaaatcagaggagccatcggtgtcaatacccttcctacaaactgcattattaagaggttcagggagtcttgggcacagaccaagccaggagatggataaaatgttaaaaaatcaagcaacttctgctacttctgaaaaggataatgatgatgaccaaagtgacaagggtacttataccattgagttagagaatcccaacagtgaggaagtggaagcaagaaaaatgattcacaaggttatcttgacgaatcagattacaacccatctgagtggagccctggcttctcag

SEQ ID NO: 7 - DNA, 905 bp, Homo sapiens
agaggtggagcctacagaggcaggcaggcctccttgagctgtggtgggccgcacccagtttgagcttcctggctgctttgtttacctaatcaagcctgggcaatggcgggtgcccctcccccagcctcgctgctgccttgcagtttgatctcagactgctgtgctagcaatcagcgagactgagccaagatggccaaataggaacagctccggtctacagctcccagcgtgagcgacgcagaagatgggtgatttctgcatttccatctgagtatgctgtctggctagtcctctgggttacgtggaatgtgtttgttatctgcttctatttggaggctggggacctctcaaaggaaacagaccttatcctgacttttaatatatcaatgcaccgatcttggtggatggagaatggaccaggatgtacggtgacgtcagtgacacctgccccagactgggccccagaagaccatcgctacatcacggtctcagggtgtttgctggagtaccagtacatagaagtggctcatagttccctccagattgtcctcgcactggcaggtttcatctacgcctgttatgttgtgaaatgtataactgaagaagaggacagctttgatttcataggtggctttgactcttatggctatcaagggcctcagaagacatctcatttacaactacagcctatgtacatgtcaaaataatacagatgacttcagtatgtcagcccatggacctttcaaagaacttttttcgcagtggcctcctgcatttcatgaagagcaagaagcaactgagtttaaatacatacacgtattaacaaaacaaatgcaaagcctctacatacaacactgacacacacacacacacacacgtgagcacgcacacaccaattccacttg

SEQ ID NO: 8 - DNA, 371 bp, Homo sapiens
acatccaaacaatttctgagaagaagtgcacatggaaattgactgtacactgtctccttggctgtgaactcaagcatgtagttgatttcataggtggctttgactcttatggctatcaagggcctcagaagacatctcatttacaactacagcctatgtacatgtcaaaataatacagatgacttcagtatgtcagcccatggacctttcaaagaacttttttcgcagtggcctcctgcatttcatgaagagcaagaagcaactgagtttaaatacatacacgtattaacaaaacaaatgcaaagcctctacatacaacactgacacacacacacacacacacgtgagcacgcacacaccaattccacttg

SEQ ID NO: 9 - DNA, 194 bp, Homo sapiens
tacagatgacttcagtatgtcagcccatggacctttcaaagaacttttttcgcagtggcctcctgcatttcatgaagagcaagaagcaactgagtttaaatacatacacgtattaacaaaacaaatgcaaagcctctacatacaacactgacacacacacacacacgtgagcacgcacacaccaattccacttg

SEQ ID NO: 10 - DNA, 833 bp, Homo sapiens
tgctagcaatcagcgagactgagccaagatggccaaataggaacagctccggtctacagctcccagcgtgagcgacgcagaagatgggtgatttctgcatttccatctgagtatgctgtctggctagtcctctgggttacgtggaatgtgtttgttatctgcttctatttggaggctggggacctctcaaaggaaacagaccttatcctgacttttaatatatcaatgcaccgatcttggtggatggagaatggaccaggatgtacggtgacgtcagtgacacctgccccagactgggccccagaagaccatcgctacatcacggtctcagggtgtttgctggagtaccagtacatagaagtggctcatagttccctccagattgtcctcgcactggcaggtttcatctacgcctgttatgttgtgaaatgtataactgaagaagaggacagctttgatttcataggtggctttgactcttatggctatcaagggcctcagaagacatctcatttacaactacagcctatgtacatgcaaattcccttggataacaactgataacaaattctatatatcagcatcaagtgatatgctgaattgcaagcgaagatcaaaatagaaagtcaaaataatacagatgacttcagtatgtcagcccatggacctttcaaagaacttttttcgcagtggcctcctgcatttcatgaagagcaagaagcaactgagtttaaatacatacacgtattaacaaaacaaatgcaaagcctctacatacaacactgacacacacacacacacacacgtgagcacgcacacaccaattccacttg

SEQ ID NO: 11 - DNA, 860 bp, Homo sapiens
agactgagccaagatggccaaataggaacagctccggtctacagctcccagcgtgagcgacgcagaagatgggtgatttctgcatttccatctgagtatgctgtctggctagtcctctgggttacgtggaatgtgtttgttatctgcttctatttggaggctggggacctctcaaaggaaacagaccttatcctgacttttaatatatcaatgcaccgatcttggtggatggagaatggaccaggatgtacggtgacgtcagtgacacctgccccagactgggccccagaagaccatcgctacatcacggtctcagggtgtttgctggagtaccagtacatagaagtggctcatagttccctccagattgtcctcgcactggcaggtttcatctacgcctgttatgttgtgaaatgtataactgaagaagaggacagctacgatgcctggacatatgacttccgatccatagagtaaaaaagttgatttcataggtggctttgactcttatggctatcaagggcctcagaagacatctcatttacaactacagcctatgtacatgcaaattcccttggataacaactgataacaaattctatatatcagcatcaagtgatatgctgaattgcaagcgaagatcaaaatagaaagtcaaaataatacagatgacttcagtatgtcagcccatggacctttcaagaacttttttcgcagtggcctcctgcatttcatgaagagcaagaagcaactgagtttaaatacatacacgtattaacaaaacaaatgcaaagcctctacatacaacactgacacacacacacacacacacgtgagcacgcacacaccaattccacttg

SEQ ID NO: 12 - DNA, 783 bp, Homo sapiens
attattatcgtcattcttggtttgtttggaactattcaatatagacctcgttacataacaggatatgctgtctggctagtcctctgggttacgtggaatgtgtttgttatctgcttctatttggaggctggggacctctcaaaggaaacagaccttatcctgacttttaatatatcaatgcaccgatcttggtggatggagaatggaccaggatgtacggtgacgtcagtgacacctgccccagactgggccccagaagaccatcgctacatcacggtctcagggtgtttgctggagtaccagtacatagaagtggctcatagttccctccagattgtcctcgcactggcaggtttcatctacgcctgttatgttgtgaaatgtataactgaagaagaggacagctttgatttcataggtggctttgactcttatggctatcaagggcctcagaagacatctcatttacaactacagcctatgtacatgcaaattcccttggataacaactgataacaaattctatatatcagcatcaagtgatatgctgaattgcgagcgaagatcaaaatagaaagtcaaaataatacagatgacttcagtatgtcagcccatggacctttcaaagaacttttttcgcagtggcctcctgcatttcatgaagagcaagaagcaactgagtttaaatacatacacgtattaacaaaacaaatgcaaagcctctacatacaacactgacacacacacacacacacgtgagcacgcacacaccaattccacttg

SEQ ID NO: 13 - DNA, 530 bp, Homo sapiens
agactgagccaagatggccaaataggaacagctccggtctacagctcccagcgtgagcgacgcagaagatgggtgatttctgcatttccatctgagtatgctgtctggctagtcctctgggttacgtggaatgtgtttgttatctgcttctatttggaggctggggacctctcaaaggaaacagaccttatcctgacttttaatatatcaatgcaccgatcttggtggatggagaatggaccaggatgtacggtgacgtcagtgacacctgccccagactgggcctcagaagacatctcatttacaactacagcctatgtacatgtcaaaataatacagatgacttcagtatgtcagcccatggacctttcaaagaacttttttcgcagtggcctcctgcatttcatgaagagcaagaagcaactgagtttaaatacatacacgtattaacaaaacaaatgcaaagcctctacatacaacactgacacacacacacacacacgtgagcacgcacacaccaattccacttg

SEQ ID NO: 14 - DNA, 381 bp, Homo sapiens
ttgatttcataggtggctttgactcttatggctatcaagggcctcagaagacatctcatttacaactacagcctatgtacatgcaaattcccttggataacaactgataacaaattctatatatcagcatcaagtgatatgctgaattgcaagcgaagatcaaaatagaaagtcaaaataatacagatgacttcagtatgtcagcccatggacctttcaaagaacttttttcgcagtggcctcctgcatttcatgaagagcaagaagcaactgagtttaaatacatacacgtattaacaaaacaaatgcaaagcctctacatacaacactgacacacacacacacacacacacgtgagcacgcacacaccaattccacttg

SEQ ID NO: 15 - DNA, 1052 bp, Homo sapiens
cttcttgttgttaacagtttccggaaagagagtgatcgggatgcaagatttttgagactcactggtctgaaactgcctttcctcttaaatttagttaagtatttccctgggcatggaattcaaaggtggttatgacttttcttcaggattgtgaatgtatttatatcctcccatcttacgttgctattgagaattctgaagttcttctgattcctgattctttgtatgtgtattcctcattccacaccttccccagaatgcatgcagaatttcttcctttctattttcttttctctttttctgaaactcttattattgggatcttctgcctcttggtttagggctctaattttccgacattttctctgctattttttactactttatttttctcctctactttctgagagatttcctcctcttgatcttccaaatcttgtactgaatcttttatttttgttaacatgttcttaatttccaagaactcttttttcttgtcttcggagtttcaacacttattgttgttttgtgcatgtattattttttcttctctctctgaggctatttaggaaattttttattgaagctcctcccccctgcttccttcaagttgcttttatctgcttttttatttgctttttcatgcaataagtttttctcacatgtctggtaactcttggggattaccaaaaactcatagaaaattctgaccatgtgagtaacacttgccaattttgagcttcatgatagaatgatctagctggaccttttgctgcggggaaatcggagtgattactttccacctgcaaagtggaaacaataagaccagttagcggctgctgctttagtcaagcaagagatgaaggtgacttggaatatgctgtacagatggcattaaatgaatatggatctccttttggaaacttttcatctgcatgatttgtaccctgttgaaatgtaaaacgactaatttaaacacttgcggtgactcagctgaaacagcttctaccaggtttgaaatgttctccctcagtggcactttcggaacccag

SEQ ID NO: 16 - DNA, 1076 bp, Homo sapiens
aacagtttccggaaagagagtgatcgggatgcaagatttttgagactcactggtctgaaactgcctttcctcttaaatttagttaagtatttccctgggcatggaattcaaaggtggttatgacttttcttcaggattgtgaatgtatttatatcctcccatcttacgttgctattgagaattctgaagttcttctgattcctgattctttgtatgtgtattcctcattccacaccttccccagaatgcatgcagaatttcttcctttctattttcttttctctttttctgaaactcttattattgggatcttctgcctcttggtttagggctctaattttccgacattttctctgctattttttactactttatttttctcctctactttctgagagatttcctcctcttgatcttccaaatcttgtactgaatcttttatttttgttaacatgttcttaatttccaagaactcttttttcttgtcttcggagtttcaacacttattgttgttttgtgcatgtattattttttcttctctctctgaggctatttaggaaattttttattgaagctcctcccccctgcttccttcaagttgcttttatctgcttttttatttgctttttcatgcaataagtttttctcacatgtctggtaactcttggggattaccaaaaactcatagaaaattctgaccatgtgagtaacacttgccaattttgagcttcatgatagaatgatctagctggaccttttgctgcggggaaatcggagttacttctatcccatcttaatacacttgcactgaagtgattactttccacctgcaaagtggaaacaataagaccagttagcggctgctgctttagtcaagcaagagatgaaggtgacttggaatatgctgtacagatggcattaaatgaatatggatctccttttggaaacttttcatctgcatgatttgtaccctgttgaaatgtaaaacgactaatttaaacacttgcggtgactcagctgaaacagcttctaccaggtttgaaatgttctccctcagtggcactttcggaacccag

SEQ ID NO: 17 - DNA, 222 bp, Homo sapiens
tttccggaaagagagtgatcgggatgcaagatttttgagactcactggtctgaaactgcctttcctcttaaatttagttaagtatttccctgggcatggaattcaaagatttgtaccctgttgaaatgtaaaacgactaatttaaacacttgcggtgacccagctgaaacagcttctaccaggtttgaaatgttctccctcagtggcactttcggaacccag

SEQ ID NO: 18 - DNA, 489 bp, Homo sapiens
gactttggagctctcagctttggagtcagttaagaccagttccttgctgggaagccctaactctggagggacagagacagtgctggccagagttacagaagttgggctgtaaacattggcaactattgacagaagtcaaggtcatggcaagaccatttggcccatgtttgttggggaaggataagcctgcgtttcaaaaagactggtcaactggatttgcattcccagaaaccaggaaatttctgaaactgagttttaaacctggcaactcttttaccatccctttgtatttttgcagttcctccctataccagcttccttacagctgttctgatttcctgtcgtaagattttttcttttcattttctccagggagggtaaatcaatataacctcttctctagagaggaggtggttaggttggtcttaagcagtgttagaagatctattttttttcaaaccaggtgtctgagctgggtgaattccagcc

SEQ ID NO: 19 - DNA, 269 bp, Homo sapiens
attcccagaaaccaggaaatttctgaaactgagttttaaacctggcaactcttttaccatccctttgtatttttgcagttcctccctataccagcttccttacagctgttctgatttcctgtcgtaagattttttcttttcattttctccagggagggtaaatcaatataacctcttctctagagaggaggtggttaggttggtcttaagcagtgttagaagatctattttttttcaaaccaggtgtctgagctgggtgaattccagcc

SEQ ID NO: 20 - DNA, 159 bp, Homo sapiens
aagtaggaagcctgggtacaggcctggcaagcgagaacaatgtgtcactgcaggactttggagctctcagctttggagtcagttaagaccagttccttgctgggaagccctaactctggagggacagagacaggtgtctgagctgggtgaattccagcc

SEQ ID NO: 21 - DNA, 399 bp, Homo sapiens
agagagggggagaggtgggggagaggagcaatggagcagaacctgagaccggagaggcaagccaggaccgctatgatccttcttaaaactagttttgaaaatgttcaaactatgtgttcgcgggtcctcgagcagaaacccaagcaactctgggacgccccccgcacccgcaagaagcgctgcccttattcgaaattccagatccgggaactggagcgagagtttttcttcaacgtgtatatcaacaaagagaagcggctgcagctgtcccggatgctgaacctgacggaccgacaagtgaaaatttggtttcagaacagaaggatgaaagaaaagaaactgagcagagaccggctgcagtatttctcgggaaatcctctgctgtaacctgcagaccgg

SEQ ID NO: 22 - DNA, 291 bp, Homo sapiens
agaggtgggggagaggagcaatggagcagaacctgagaccggagagacgccccccgcacccgcaagaagcgctgcccttattcgaaattccagatccgggaactggagcgagagtttttcttcaacgtgtatatcaacaaagagaagcggctgcagctgtcccggatgctgaacctgacggaccgacaagtgaaaatttggtttcagaacagaaggatgaaagaaaagaaactgagcagagaccggctgcagtatttctcgggaaatcctctgctgtaacctgcagaccgg

SEQ ID NO: 23 - DNA, 381 bp, Homo sapiens
agcttcgcccagaaggtaagtgtccctggacagagggagggcttgagcccgggaaccggagggtcggactgccagtgagaatcgttcagggtgctgtgagagggagacaggaaagaaccatgtggctggatggtaagaaaatataagctgggcgcgatggttctcacctgcaatcccagcactttgggaggccaagacaggaggatcgcttgaggccaggaattcaaggcaacacggtgaccaatgctcaggacttcggggctcaaggagtgctcatatacccagagccagcggacttctcccaggacccacccaagccaagcctgtccagccagcaggcagtgtatggacatgtgcacctgggaactggagacccctaca

SEQ ID NO: 24 - DNA, 333 bp, Homo sapiens
agtgggccgcctgctgctggtgcgcgtgggggtgatcagcttcgcccagaaggtgaccaatgctcaggacttcggggctcaaggagtgctcatatacccagagccagcggacttctcccaggacccacccaagccaagcctgtccagccagcaggcagtgtatggacatgtgagtctggggaggctggtcgtgctgttcccaggtcccgaggccacaccttcttgctcagaatagggctggcatgtttttcgtttttcctggaagcccagctcgcagctttgcgtgggcacccctttccctgcaggtgcacctgggaactggagacccctaca

SEQ ID NO: 25 - DNA, 363 bp, Homo sapiens
gggggtgatcagcttcgcccagaagcactttaggaggccaagacaggaggatcgcttgaggccaggaattcaaggcaacacggtgaccaatgctcaggacttcggggctcaaggagtgctcatatacccagagccagcggacttctcccaggacccacccaagccaagcctgtccagccagcaggcagtgtatggacatgtgagtctggggaggctggtcgtgctgttcccaggtcccgaggccacaccttcttgctcagaatagggctgacatgtttttcgtttttcctggaagcccagctcgcagctttgcgtgggcacccctttccctgcaggtgcacctgggaactggagacccctaca

SEQ ID NO: 26 - DNA, 578 bp, Homo sapiens
cctcttccctccctctcctccgggcgcccagaaaagtccccacctctccccgcttaggcaaaccagccttcgggaacgggtggcaggctcggccgggatggccgctctgactcaggacattcgcgcggcgctctcccgccagaagctggaccacgtgtggaccgacacgcactacgtggggctgcaattcccggatccggctcaccccaacaccctgcactgggtcgatgaggccgggaaggtcggagagcagctgccgctggaggaccctgacgtctactgcccctacagcgccatcggcaacgtcacgggagagctggtgtacgcccactacgggcggcccgaagacctgcaggacctgcgggccaggggcgtggatccagtgggccgcctgctgctggtgcgcgtgggggtgatcagcttcgcccagaaggtgaccaatgctcaggacttcggggctcaaggagtgctcatatacccagagccagcggacttctcccaggacccacccaagccaagcctgtccagccagcaggcagtgtatggacatgtgcacctgggaactggagacccctaca

SEQ ID NO: 27 - DNA, 103 bp, Homo sapiens
tacccagagccagcggacttctcccaggacccacccaagccaagcctgtccagccagcaggcagtgtatggacatgtgcacctgggaactggagacccctaca

SEQ ID NO: 28 - DNA, 292 bp, Homo sapiens
gcctcaccgtcctcgtgtccccagggagagctggtgtacgcccactacgggcggcccgaagacctgcaggacctgcgggccaggggcgtggatccagtgggccgcctgctgctggtgcgcgtgggggtgatcagcttcgcccagaaggtgaccaatgctcaggacttcggggctcaaggagtgctcatatacccagagccagcggacttctcccaggacccacccaagccaagcctgtccagccagcaggcagtgtatggacatgtgcacctgggaactggagacccctaca

SEQ ID NO: 29 - DNA, 77 bp, Homo sapiens
gggccgcctgctgctggtgcgcgtgggggtgatcagcttcgcccagaaggtgcacctgggaactggagacccctaca

SEQ ID NO: 30 - DNA, 194 bp, Homo sapiens
gggccgcctgctgctggtgcgcgtgggggtgatcagcttcgcccagaaggtgaccaatgctcaggacttcggggctcaaggagtgctcatatacccagagccagcggacttctcccaggacccacccaagccaagcctgtccagccagcaggcagtgtatggacatgtgcacctgggaactggagacccctaca

SEQ ID NO: 31 - DNA, 128 bp, Homo sapiens
ggggcgtggatccagtgggccgcctgctgctggtgcgcgtgggggtgatcagcttcgcccagaagctcgcagctttgcgtgggcacccctttccctgcaggtgcacctgggaactggagacccctaca

SEQ ID NO: 32 - DNA, 239 bp, Homo sapiens
tacccagagccagcggacttctcccaggacccacccaagccaagcctgtccagccagcaggcagtgtatggacatgtgagtctggggaggctggtcgtgctgttcccaggtcccgaggccacaccttcttgctcagaatagggctggcatgtttttcgtttttcctggaagcccagctcgcagctttgcgtgggcacccctttccctgcaggtgcacctgggaactggagacccctaca

SEQ ID NO: 33 - DNA, 743 bp, Homo sapiens
agtcatagggaagccatcccagaagccaggtctactcatcaataagcagctgcctgtgcagagtgcaggctgcacctttggacagcctttaaaactgaattctcagaattttagaacaaatttttgtctagaaatgctgactttggttcattaggtagtggtaaaacaggctcccttcgaagctctccttcatcaccttcctaagtgcatgggggaaaatacctagggctcaacagtcttgagaagtgtggaaacattttctttgtgagtgagaacagatcacctagagaaaggaaaccagattcccatcactgcttctgggtatcagatgctagcgctgcactccattttgcaatggcctcccttgctgcagcaaatgcagagttttgcttcaacctgttcagagagatggatgacaatcaaggaaatggaaatgtgttcttttcctctctgagcctcttcgctgccctggccctggtccgcttgggcgctcaagatgactccctctctcagattgataagttgcttcatgttaacactgcctcaggatatggaaactcttctaatagtcagtcagggctccagtctcaactgaaaagagttttttctgatataaatgcatcccacaaggattatgatctcagcattgtgaatgggctttttgctgaaaaagtgtatggctttcataaggactacattgagtgtgccgaaaattatacgatgccaaagtggagcgagttgac

SEQ ID NO: 34 - DNA, 453 bp, Homo sapiens
agactcacccaaccagcggccaacttcctctatgaagacagtaaggctgcactccattttgcaatggcctcccttgctgcagcaaatgcagagttttgcttcaacctgttcagagagatggatgacaatcaaggaaatggaaatgtgttcttttcctctctgagcctcttcgctgccctggccctggtccgcttgggcgctcaagatgactccctctctcagattgataagttgcttcatgttaacactgcctcaggatatggaaactcttctaatagtcagtcagggctccagtctcaactgaaaagagttttttctgatataaatgcatcccacaaggattatgatctcagcattgtgaatgggctttttgctgaaaaagtgtatggctttcataaggactacattgagtgtgccgaaaaattatacgatgccaaagtggagcgagttgac

SEQ ID NO: 35 - DNA, 178 bp, Homo sapiens
tagtcagtcagggctccagtctcaactgaaaagagttttttctgatataaatgcatcccacaaggattatgatctcagcattgtgaatgggctttttgctgaaaaagtgtatggctttcataaggactacatcgagtgtgccgaaaaattatacgatgccaaagtggagcgagttgac

SEQ ID NO: 36 - DNA, 145 bp, Homo sapiens
ctcagggctccgggagcgagatgaagaggaagaggcagcccggggtcggcggcggcgccagcgggaattaaatcgcagaaagtaccaggcactaggtcggcgctgccgggagatcgagcaggtgaacgagcgggtcctgaacagg

SEQ ID NO: 37 - DNA, 382 bp, Homo sapiens
agttttgcgaacggcttaacctacagattgaagaggtcggaagctctgaggcccggggcttccggaggtcgcggagatggaattggagcagagagaagggaccatggcagccgtgggctttgaggagttctcagcgccgccaggctcagagttggcgttgcctcccctatttggtggccacatcctggagagcgagctggagacggaagtggagtttgtgtcaggtggtctgggcggctcagggctccgggagcgagatgaagaggaagaggcagcccggggtcggcggcggcgccagcgggaattaaatcgcagaaagtaccaggcactaggtcggcgctgccgggagatcgagcaggtgaacgagcgggtcctgaacagg

SEQ ID NO: 38 - DNA, 456 bp, Homo sapiens
gtcatctttattttccgagtcatgatcctcgtggtggctgcccaggaagtgtggggtgacgagcaagaggacttcgtctgcaacacactgcaaccgggatgcaaaaatgtgtgctatgaccactttttcccggtgtcccacatccggctgtgggccctccagctgatcttcgtctccaccccagcgctgctggtggccatgcatgtggcctactacaggcacgaaaccactcgcaagttcaggcgaggagagaagaggaatgatttcaaagacatagaggacattaaaaagcagaaggttcggatagaggggtcgctgtggtggacgtacaccagcagcatctttttccgaatcatctttgaagcagcctttatgtatgtgttttacttcctttacaatgggtaccacctgccctgggtgttgaaatgtgggattgacccctgccccaaccttgtt

SEQ ID NO: 39 - DNA, 865 bp, Homo sapiens
agggccgccggccgcagagcagttaaaacgtgcaggcaccagaaggcacttcctgtcggtgaagaagacctgtctccggtgtcacgggcatcctgtgttttgcaaacggggctgacctcccttcctggggagcaggaagggtcagggcgacatcctcactgaccgagcaaagattgacattcgtatcatcactgtgcaccattggcttctaggcactccagtggggtaggagaaggaggtctgaaaccctcgcagagggatcttgccctcattctttgggtctgaaacactggcagtcgttggaaacaggactcagggataaaccagcgcaatggattgggggacgctgcacactttcatcgggggtgtcaacaaacactccaccagcatcgggaaggtgtggatcacagtcatctttattttccgagtcatgatcctcgtggtggctgcccaggaagtgtggggtgacgagcaagaggacttcgtctgcaacacactgcaaccgggatgcaaaaatgtgtgctatgaccactttttcccggtgtcccacatccggctgtgggccctccagctgatcttcgtctccaccccagcgctgctggtggccatgcatgtggcctactacaggcacgaaaccactcgcaagttcaggcgaggagagaagaggaatgatttcaaagacatagaggacattaaaaagcagaaggttcggatagaggggtcgctgtggtggacgtacaccagcagcatctttttccgaatcatctttgaagcagcctttatgtatgtgttttacttcctttacaatgggtaccacctgccctgggtgttgaaatgtgggattgacccctgccccaaccttgtt

SEQ ID NO: 40 - DNA, 741 bp, Homo sapiens
agggccgccggccgcagagcaggcgacatcctcactgaccgagcaaagattgacattcgtatcatcactgtgcaccattggcttctaggcactccagtggggtaggagaaggaggtctgaaaccctcgcagagggatcttgccctcattctttgggtctgaaacactggcagtcgttggaaacaggactcagggataaaccagcgcaatggattgggggacgctgcacactttcatcgggggtgtcaacaaacactccaccagcatcgggaaggtgtggatcacagtcatctttattttccgagtcatgatcctcgtggtggctgcccaggaagtgtggggtgacgagcaagaggacttcgtctgcaacacactgcaaccgggatgcaaaaatgtgtgctatgaccactttttcccggtgtcccacatccgactgtgggccctccagctgatcttcgtctccaccccagcgctgctggtggccatgcatgtggcctactacaggcacgaaaccactcgcaagttcaggcgaggagagaagaggaatgatttcaaagacatagaggacattaaaaagcagaaggttcggatagaggggtcgctgtggtggacgtacaccagcagcatctttttccgaatcatctttgaagcagcctttatgtatgtgttttacttcctttacaatgggtaccacctgccctgggtgttgaaatgtgggattgacccctgccccaaccttgtt

SEQ ID NO: 41 - DNA, 889 bp, Homo sapiens
gcgccagggcgccggccgcagagcagttaaaacgtgcaggcaccagaaggcacttcctgtcggtgaagaagacctgtctccggtgtcacgggcatcctgtgttttgcaaacggggctgacctcccttcctggggagcaggaagggtcaggctactatttttccctcaacaggcgacatcctcactgaccgagcaaagattgacattcgtatcatcactgtgcaccattggcttctaggcactccagtggggtaggagaaggaggtctgaaaccctcgcagagggatcttgccctcattctttgggtctgaaacactggcagtcgttggaaacaggactcagggataaaccagcgcaatggattgggggacgctgcacactttcatcgggggtgtcaacaaacactccaccagcatcgggaaggtgtggatcacagtcatctttattttccgagtcatgatcctcgtggtggctgcccaggaagtgtggggtgacgagcaagaggacttcgtctgcaacacactgcaaccgggatgcaaaaatgtgtgctatgaccactttttcccggtgtcccacatccggctgtgggccctccagctgatcttcgtctccaccccagcgctgctggtggccatgcatgtggcctactacaggcacgaaaccactcgcaagttcaggcgaggagagaagaggaatgatttcaaagacatagaggacattaaaaagcagaagttcggatagaggggtcgctgtggtggacgtacaccagcagcatctttttccgaatcatctttgaagcagcctttatgtatgtgttttacttcctttacaatgggtaccacctgccctgggtgttgaaatgtgggattgacccctgccccaaccttgtt

SEQ ID NO: 42 - DNA, 795 bp, Homo sapiens
tgtctccggtgtcacgggcatcctgtgttttgcaaacggggctgacctcccttcctggggagcaggaagggtcagggcgacatcctcactgaccgagcaaagattgacattcgtatcatcactgtgcaccattggcttctaggcactccagtggggtaggagaaggaggtctgaaaccctcgcagagggatcttgccctcattctttaggtctgaaacactggcagtcgttggaaacaggactcagggataaaccagcgcaatggattgggggacgctgcacactttcatcgggggtgtcaacaaacactccaccagcatcgggaaggtgtggatcacagtcatctttattttccgagtcatgatcctcgtggtggctgcccaggaagtgtggggtgacgagcaagaggacttcgtctgcaacacactgcaaccgggatgcaaaaatgtgtgctatgaccactttttcccggtgtcccacatccggctgtgggccctccagctgatcttcgtctccaccccagcgctgctggtggccatgcatgtggcctactacaggcacgaaaccactcgcaagttcaggcgaggagagaagaggaatgatttcaaagacatagaggacattaaaaagcagaaggttcggatagaggggtcgctgtggtggacgtacaccagcagcatctttttccgaatcatctttgaagcagcctttatgtatgtgttttacttcctttacaatgggtaccacctgccctgggtgttgaaatgtgggattgacccctgccccaaccttgtt

SEQ ID NO: 43 - DNA, 764 bp, Homo sapiens
ggagttaaaggaatatggcaacagctactatttttccctcaacaggcgacatcctcactgaccgagcaaagattgacattcgtatcatcactgtgcaccattggcttctaggcactccagtggggtaggagaaggaggtctgaaaccctcgcagagggatcttgccctcattctttgggtctgaaacactggcagtcgttggaaacaggactcagggataaaccagcgcaatggattgggggacgctgcacactttcatcgggggtgtcaacaaacactccaccagcatcgggaaggtgtggatcacagtcatctttattttccgagtcatgatcctcgtggtggctgcccaggaagtgtggggtgacgagcaagaggacttcgtctgcaacacactgcaaccgggatgcaaaaatgtgtgctatgaccactttttcccggtgtcccacatccggctgtgggccctccagctgatcttcgtctccaccccagcgctgctggtggccatgcatgtggcctactacaggcacgaaaccactcgcaagttcaggcgaggagagaagaggaatgatttcaaagacatagaggacattaaaaagcagaaggttcggatagaggggtcgctgtggtggacgtacaccagcagcatctttttccgaatcatctttgaagcagcctttatgtatgtgttttacttcctttacaatgggtaccacctgccctgggtgttgaaatgtgggattgacccctgccccaaccttgtt

SEQ ID NO: 44 - DNA, 787 bp, Homo sapiens
attctaagcttcaagggtattcctggtaaggaagcattgatgaagaaggattctcttagcaaaagacttagaggcctcagttgtcaggcctctggatttaagctacattttgactctgtacacaaatctagatcattccctcaatttgtaataaaaacaatattatagctgaggtaggtgtcatcatgacttgtgctaatagttttgctgagtacattgtgtgtttataatcagagagaatcaggagaatttgcatattagaagagagagccaaaagtactttctaaatagtgaatggccaagctctccttgttctccccatcctcactccaacacttcataattttctttgggctgagcctctttggggaaaagctatggggaatgtaagtcaagttcattagatggagcatgtgacttgggttttggcagcatggtgttgcaaatctctgggacctacacaggctctgcagagctcaggccttagggtgatagtttgattgaggtgtcagcacagctggtattccattcagtttcacatatacatcactcctgactgggctgggtgggggcagcaggagaggagcatactacactctgactgctatgggcagcctggacaattatctcaccacgctaccttcaggggaactcagagaaaaccaaggaaggatcttgacagatgaccagctgaactcagaagaaaaaaagaagagaagcagcgaagaatagacaaccttcaatagcagccagctgcagctttgggagcgtgtctttgagcggacac

SEQ ID NO: 45 - DNA, 629 bp, Homo sapiens
ggaatctctaaggttcctcccacaggatgcagagcagaagagggtgaggggtgttggagttagctgaagcttgcatggcaagagcaaggccttagtgaatagggatcaccacatgctgtcaggtccccatgcttgaggatggcactttatgccttcttctccaccttccctctagggcaacttcagcagccacatgaggagtccagagtgaacttcatactagagccgagagcctgagaaatcaatatgaattggtgctatcagaaaaggtttatagagaagaaacagtcttctaaggacaagaggagtttcacgttttccctgaaaacaaactcagcaagcatttactatatctgggtgctttaattatcaagaagaaatgtgcagtctctattctaagcttcaagggtattcctggagaggagcatactacactccgactgctatggacaacctggacaattatctcaccacgctaccttcaggggaactcagagaaaaccaaggaaggatcttgacagatgaccagctgaactcagaagaaaaaaagaagagaaagcagcgaaggaataggacaaccttcaatagcagccagctgctggctttggagcgtgtctttgagcggacac

SEQ ID NO: 46 - DNA, 1056 bp, Homo sapiens
atggcaagagcaaggccttagtgaatagggatcaccacatgctgtcaggtccccatgcttgaggatggcactttatgccttcttctccaccttccctctagggcaacttcagcagccacatgaggagtccagagtgaacttcatactagagccgagaaacagtcttctaaggacaagaggagtttcacgttttccctgaaaacaaactcagcaagcatttactatatctgggtgctttaattatcaagaagaaatgtgcagtctctattctaagcttcaagggtattcctggtaaggaagcattgatgaagaaggattctcttagcaaaagacttagaggcctcagttgtcaggcctctggatttaagctacattttgactctgtacacaaatctagatcattccctcaatttgtaataaaaacaatattatagctgaggtaggtgtcatcatgacttgtgctaatagttttgctgagtacattgtgtgtttataatcagagagaatcaggagaatttgcatattagaagagagagccaaaagtactttctaaatagtgaatggccaagctctccttgttctccccatcctcactccaacacttcataattttctttgggctgagcctctttggggaaaagctatggggaatgtaagtcaagttcattagatggagcatgtgacttgggttttggcagcatggtgctgcaaatctctgggacctacacaggctctgcagagctcaggccttagggtgatagtttgattgaggtgtcagcacagctggtattccattcagtttcacatatacatcactcctgactgggctgggtgggggcagcaggagaggagcatactacactctgactgctatggacaacctggacaattatctcaccacgctgccttcaggggaactcagagaaaaccaaggaaggatcttgacagatgaccagctgaactcagaagaaaaaaagaagagaaagcagcgaaggaataggacaaccttcaatagcagccagctgcaggctttggagcgtgtctttgagcggacac

SEQ ID NO: 47 - DNA, 302 bp, Homo sapiens
agggcaacttcagcagccacatgaggagtccagagtgaacttcatactagagccgagaaacagtcttctaaggacaagaggagtttcacgttttccctgaaaacaaactcagcaagcatttactatatctgggtgctttaattatcaagaagaaatgtgcagtctctattctaagcttcaagggtattcctgatgaccagctgaactcagaagaaaaaaaaagaagagaaagcagcgaaggaataggacaaccttcaatagcagccagctgcaggctttggagcgtgtctttgagcggacac

SEQ ID NO: 48 - DNA, 277 bp, Homo sapiens
agctgaagcttgcatggcaagagcaaggccttagtgaatagggatcaccacatgctgtcaggtccccatgcttgaggatggcactttatgccttcttctccaccttccctctagggcaacttcagcagccacatgaggagtccagagtgaacttcatactagagccgagatgaccagctgaactcagaagaaaaaaagaagagaaagcagcgaaggaataggacaaccttcaatagcagccagctgcaggctttggagcgtgtctttgagcggacac

SEQ ID NO: 49 - DNA, 477 bp, Homo sapiens
acatgctgtcaggtccccatgcttgaggatggcactttatgccttcttctccaccttccctctagggcaacttcagcagccacatgaggagtccagagtgaacttcatactagagccgagagcctgagaaatcaatatgaattggtgctatcagaaaaggtttatagagaagatgcagctgatcttacattcttactgagtgtcacagaagtgccaccaaagctgggccctgagaaacagtcttctaaggacaagaggagtttcacgttttccctgaaaacaaactcagcaagcatttactatatctgggtgctttaattatcaagaagaaatgtgcagtctctattctaagcttcaagggtattcctgatgaccagctgaactcagaagaaaaaaagaagagaaagcagcgaaggaataggacaaccttcaatagcagccagctgcaggctttggagcgtgtctttgagcggacac

SEQ ID NO: 50 - DNA, 499 bp, Homo sapiens
gcagacttctgttttgggggcatgaaattcagataattgtgctatcacaagggcttgtattacttagaaagacctgaagtacgagacaaaatgatttggaaaaatgaataggaatgggaggaatgcttccaaatattaaataggaaggagaatgcaagtgcaatgtaactactgaaaggtatttaatcctaggtgtttgaattctagtgctgatcttattgcaaactaattttgctaatagctagtggtataatcagtgatgttggttggaatattttaggcattggtagatctcaaaaattattattaaatttgctccatttccaagatgcagctgatcttacattcttactgagtgtcacagaagtgccaccaaagctgggccctgagatgaccagctgaactcagaagaaaaaaaagaagagaaagcagcgaaggaataggacaaccttcaatagcagccagctgcaggctttggagcgtgtctttgagcggacac

SEQ ID NO: 51 - DNA, 650 bp, Homo sapiens
agtgtctaattggtctcagttaaaacaaagtacagaagctaaggatttttctttttttttttgcagacttctgttttgggggcatgaaattcagataattgtgctatcacaagggcttgtattacttagaaagacctgaagtacgagacaaaatgatttggaaaaatgaataggaatgggaggaatgcttccaaatattaaataggaaggagaatgcaagtgcaatgtaactactgaaagatgcagctgatcttacattcttactgagtgtcacagaagtgccaccaaagctgggccctgagaaacagtcttctaaggacaagaggagtttcacgttttccctgaaaacaaactcagcaagcatttactatatctgggtgctttaattatcaagaagaaatgtgcagtctctattctaagcttcaagggtattcctggagaggagcatactacactctgactgctatggacaacctggacaattatctcaccacgctaccttcaggggaactcagagaaaaccaaggaaggatcttgacagatgaccagctgaactcagaagaaaaaaaagaagagaaagcagcgaaggaataggacaaccttcaatagcagccagctgcaggctttggagcgtgtctttgagcggacac

SEQ ID NO: 52 - DNA, 175 bp, Homo sapiens
tatctgggtgctttaattatcaagaagaaatgtgcagtctctattctaagcttcaagggtattcctgatgaccagctgaactcagaagaaaaaaagaagagaaagcagcgaaggaataggacaaccttcaatagcagccagctgcaggctttggagcgtgtctttgagcggacac

SEQ ID NO: 53 - DNA, 406 bp, Homo sapiens
ctccccggcccaccgccctgagtccagattatctctcctggacagcctcgtccccctacagcacagtgccaccctacagccctgggagctcaggccccgcaaccccaggggtcaacatggccaacagcatcgccagcctccgtctcaaggccaaggagttcagcctgcaccacagccaggtgcctacggtgaactgaagtccagtcccaccaggacccagacgcctccctgggtggacagcaatagaaaagggggcagacgcccaggaagtgaccttctcctggatgagctctcctggcccgtctgtccagcctggactcccgagcccacgaggctgttgaggcccctgcagccgggcccagctcttctgtccttggccaccagagactgcagcccacaacccttg

SEQ ID NO: 54 - DNA, 233 bp, Homo sapiens
agccaggtgcctacggtgaactgaagtccagtcccaccaggacccagacgcctccctggatggacagcaatagaaaagggggcagacgcccaggaagtgaccttctcctggatgagctctcctggcccgtctgtccagcctggactcccgagcccacgaggctgttgaggcccctgcagccgggcccagctcttctgtccttggccaccagagactgcagcccacaacccttg

SEQ ID NO: 55 - DNA, 27 bp, Artificial Sequence
  synthetic oligonucleotide, RAD51L1_ex5_F
cccaccaacatgggaggattagaagga

SEQ ID NO: 56 - DNA, 25 bp, Artificial Sequence
  synthetic oligonucleotide, RAD51L1_ex8_R
agctggagacaccaggtctgcctga

SEQ ID NO: 57 - DNA, 25 bp, Artificial Sequence
  synthetic oliognucleotide, RAD51L1_ex8_nest_R
ctgagaagccagggctccactcaga

SEQ ID NO: 58 - DNA, 27 bp, Artificial Sequence
  synthetic oligonucleotide, NKAIN2_ex8_F
tggctatcaagggcctcagaagacatc

SEQ ID NO: 59 - DNA, 25 bp, Artificial Sequence
  synthetic oligonucleotide, NKAIN2_ex10_R
caggaaatccaagatgggcgtgtcc

SEQ ID NO: 60 - DNA, 25 bp, Artificial Sequence
  synthetic oligonucleotide, NKAIN2_ex10_nest_R
caagtggaattggtgtgtgcgtgct

SEQ ID NO: 61 - DNA, 25 bp, Artificial Sequence
  synthetic oligonucleotide, VNN1_ex5_F
tgcacactgtggaagggcgctatta

SEQ ID NO: 62 - DNA, 27 bp, Artificial Sequence
  synthetic oligonucleotide, VNN1_ex7_R
ggcttcagactaaacaagcgtccgtca

SEQ ID NO: 63 - DNA, 25 bp, Artificial Sequence
  synthetic oligonucleotide, VNN1_ex6_nest_R
ctgggttccgaaagtgccactgagg

SEQ ID NO: 64 - DNA, 26 bp, Artificial Sequence
  synthetic oligonucleotide, C4BPB_ex1_F
ccttgctgggaagccctaactctgga

SEQ ID NO: 65 - DNA, 25 bp, Artificial Sequence
  synthetic oligonucleotide, C4BPB_ex2_R
acgcaaccataagacagcacgcaca

SEQ ID NO: 66 - DNA, 25 bp, Artificial Sequence
  synthetic oligonucleotide, C4BPB_ex2_nest_R
ggctggaattcacccagctcagaca

SEQ ID NO: 67 - DNA, 25 bp, Artificial Sequence
  synthetic oligonucleotide, HOXC11_ex1_F
acaaatcccagctcgtccggttcag

SEQ ID NO: 68 - DNA, 25 bp, Artificial Sequence
  synthetic oligonucleotide, HOXC11_ex2_R
ccctggccacagtccagttttccac

SEQ ID NO: 69 - DNA, 25 bp, Artificial Sequence
  synthetic oligonucleotide, HOXC11_ex2_nest_R
ccggtctgcaggttacagcagagga

SEQ ID NO: 70 - DNA, 25 bp, Artificial Sequence
  synthetic oligonucleotide, TFR2_ex7_F
tcaggacttcggggctcaaggagtg

SEQ ID NO: 71 - DNA, 25 bp, Artificial Sequence
  synthetic oligonucleotide, TFR2_ex8_R
gctgggaaggcctgatgatgcaact

SEQ ID NO: 72 - DNA, 25 bp, Artificial Sequence
  synthetic oligonucleotide, TFR2_ex8_nest_R
tgtaggggtctccagttcccaggtg

SEQ ID NO: 73 - DNA, 25 bp, Artificial Sequence
  synthetic oligonucleotide, SERPINB7_ex3_F
ttgggcgctcaagatgactccctct

SEQ ID NO: 74 - DNA, 25 bp, Artificial Sequence
  synthetic oligonucleotide, SERPINB7_ex5_R
gtcaactcgctccactttggcatcg

SEQ ID NO: 75 - DNA, 26 bp, Artificial Sequence
  synthetic oligonucleotide, SERPINB7_ex6_R
gaaggctgattgccacttgcctttga

SEQ ID NO: 76 - DNA, 25 bp, Artificial Sequence
  synthetic oligonucleotide, TFPT_ex3_F
cacatcctggagagcgagctggaga

SEQ ID NO: 77 - DNA, 25 bp, Artificial Sequence
  synthetic oligonucleotide, TFPT_ex4_R
tcctgctgcagcctccgagttatcc

SEQ ID NO: 78 - DNA, 24 bp, Artificial Sequence
  synthetic oligonucleotide, TFPT_ex4_nest_R
cctgttcaggacccgctcgttcac

SEQ ID NO: 79 - DNA, 25 bp, Artificial Sequence
  synthetic oligonucleotide, GJB6_ex3_F
ttcggatagaggggtcgctgtggtg

SEQ ID NO: 80 - DNA, 25 bp, Artificial Sequence
  synthetic oligonucleotide, GJB6_ex3_R
gcagcatgcaaatcacagacgcaga

SEQ ID NO: 81 - DNA, 25 bp, Artificial Sequence
  synthetic oligonucleotide, GJB6_ex3_nest_R
aacaaggttggggcaggggtcaatc

SEQ ID NO: 82 - DNA, 25 bp, Artificial Sequence
  synthetic oligonucleotide, PRRX1_ex_F
tagacctggaggaagccggggacat

SEQ ID NO: 83 - DNA, 25 bp, Artificial Sequence
  synthetic oligonucleotide, PRRX1_ex_R
taatcggtgggtctcggagcaggac

SEQ ID NO: 84 - DNA, 25 bp, Artificial Sequence
  synthetic oligonucleotide, PRRX1_ex_nest_R
gtgtccgctcaaagacacgctccaa

SEQ ID NO: 85 - DNA, 25 bp, Artificial Sequence
  synthetic oligonucleotide, PRRX1_int1_ex3_R
cccagctttggtggcacttctgtga

SEQ ID NO: 86 - DNA, 28 bp, Artificial Sequence
  synthetic oligonucleotide, PRRX1_int1_ex4_R
tcagggaaaacgtgaaactcctcttgtc

SEQ ID NO: 87 - DNA, 25 bp, Artificial Sequence
  synthetic oligonucleotide, PRRX2_ex3_F
gcccaccgccctgagtccagattat

SEQ ID NO: 88 - DNA, 25 bp, Artificial Sequence
  synthetic oligonucleotide, PRRX2_ex4_R
aggtccttggcaggctcttccacct

SEQ ID NO: 89 - DNA, 25 bp, Artificial Sequence
  synthetic oligonucleotide, PRRX2_ex4_nest_R
caagggttgtgggctgcagtctctg

SEQ ID NO: 90 - DNA, 20 bp, Artificial Sequence
  synthetic oligonucleotide, ABL1_ex2_rev
accctgcggctcaaagtcag

SEQ ID NO: 91 - DNA, 23 bp, Artificial Sequence
  synthetic oligonucleotide, ABL1_ex3_rev
ttccccattgtgattatagccta

SEQ ID NO: 92 - DNA, 20 bp, Artificial Sequence
  synthetic oligonucleotide, BCR_ex1_forw
caacagtccttcgacagcag

SEQ ID NO: 93 - DNA, 21 bp, Artificial Sequence
  synthetic oligonucleotide, BCR_ex13_forw
cagatgctgaccaactcgtgt

SEQ ID NO: 94 - DNA, 20 bp, Artificial Sequence
  synthetic oligonucleotide, BIRC5-6′FAM-F
tctccgcagtttcctcaaat

SEQ ID NO: 95 - DNA, 19 bp, Artificial Sequence
  synthetic oligonucleotide, BIRC5-EX2-L-F
gaggctggcttcatccact

SEQ ID NO: 96 - DNA, 20 bp, Artificial Sequence
  synthetic oligonucleotide, BIRC5-EX1- F
agaactggcccttcttggag

SEQ ID NO: 97 - DNA, 20 bp, Artificial Sequence
  synthetic oligonucleotide, BIRC5-EX2-K-F
gcccagtgtttcttctgctt

SEQ ID NO: 98 - DNA, 20 bp, Artificial Sequence
  synthetic oligonucleotide, BIRC5-ex4_Rev
tctccgcagtttcctcaaat

SEQ ID NO: 99 - DNA, 25 bp, Artificial Sequence
  synthetic oligonucleotide, CST_ex1_F
tgcgggtactaagagccaggcaaca

SEQ ID NO: 100 - DNA, 24 bp, Artificial Sequence
  synthetic oligonucleotide, CST_ex3_R
cgaatggcctggcacagatcccta

SEQ ID NO: 101 - DNA, 27 bp, Artificial Sequence
  synthetic oligonucleotide, CST_ex3_nest_R
tgacacctggatttcaccagggacctt

SEQ ID NO: 102 - DNA, 25 bp, Artificial Sequence
  synthetic oligonucleotide, FZD10_ex1_F
tttatgctgctggtggtggggatca

SEQ ID NO: 103 - DNA, 25 bp, Artificial Sequence
  synthetic oligonucleotide, FZD10_ex1_R
gcgtggtgagttttctggggatgct

SEQ ID NO: 104 - DNA, 25 bp, Artificial Sequence
  synthetic oligonucleotide, FZD10_ex1_nest_R
gccgccaggatcttccagtaatcca

SEQ ID NO: 105 - DNA, 20 bp, Artificial Sequence
  synthetic oligonucleotide, GPR177_ex2_R
ggaggggaatgtgaacagaa

SEQ ID NO: 106 - DNA, 20 bp, Artificial Sequence
  synthetic oligonucleotide, GPR177_ex1_F
tctgctcgtgttccaaatca

SEQ ID NO: 107 - DNA, 25 bp, Artificial Sequence
  synthetic oligonucleotide, HOXB13_ex1_F
cagccagatgtgttgccagggagaa

SEQ ID NO: 108 - DNA, 25 bp, Artificial Sequence
  synthetic oligonucleotide, HOXB13_ex2_R
cttgcgcctcttgtccttggtgatg

SEQ ID NO: 109 - DNA, 28 bp, Artificial Sequence
  synthetic oligonucleotide, HOXB13_ex2_R_alt2
taaggggtagcgctgttcttcaccttgg

SEQ ID NO: 110 - DNA, 20 bp, Artificial Sequence
  synthetic oligonucleotide, Hs.446400_F
cagagctgcatccttatggt

SEQ ID NO: 111 - DNA, 20 bp, Artificial Sequence
  synthetic oligonucleotide, Hs.446400_R
agctgcaagttgttgttcca

SEQ ID NO: 112 - DNA, 22 bp, Artificial Sequence
  synthetic oligonucleotide, MIER1_ex9_F
ccatcagaagactggaaaaagg

SEQ ID NO: 113 - DNA, 22 bp, Artificial Sequence
  synthetic oligonucleotide, MIER1_ex10_R
tgcttctacacccttctcatca

SEQ ID NO: 114 - DNA, 20 bp, Artificial Sequence
  synthetic oligonucleotide, MTHFD2L_ex5_F
gacccaagagtcagcggtat

SEQ ID NO: 115 - DNA, 20 bp, Artificial Sequence
  synthetic oligonucleotide, MTHFD2L_ex7_R
gatcttccagccacaaccac

SEQ ID NO: 116 - DNA, 21 bp, Artificial Sequence
  synthetic oligonucleotide, PBX1_ex3_rev
tgctccactgagttgtctgaa

SEQ ID NO: 117 - DNA, 20 bp, Artificial Sequence
  synthetic oligonucleotide, PBX1_ex5_rev
gggttgctgagatgggaata

SEQ ID NO: 118 - DNA, 20 bp, Artificial Sequence
  synthetic oligonucleotide, RUNX1_ex2_rev
cgtggacgtctctagaagga

SEQ ID NO: 119 - DNA, 19 bp, Artificial Sequence
  synthetic oligonucleotide, TCF3_ex_15_forw
caccctccctgacctgtct

SEQ ID NO: 120 - DNA, 20 bp, Artificial Sequence
  synthetic oligonucleotide, TCF3_ex_17_forw
gtgacatcaacgaggccttt

SEQ ID NO: 121 - DNA, 23 bp, Artificial Sequence
  synthetic oligonucleotide, NUP
aagcagtggtatcaacgcagagt

SEQ ID NO: 122 - DNA, 45 bp, Artificial Sequence
  synthetic oligonucleotide, UPM - Long
ctaatacgactcactatagggcaagcagtggtatcaacgcagagt

SEQ ID NO: 123 - DNA, 22 bp, Artificial Sequence
  synthetic oligonucleotide, UPM - Short
ctaatacgactcactatagggc

SEQ ID NO: 124 - DNA, 18 bp, Artificial Sequence
  synthetic oligonucleotide, USP11_eks6_R
gcctggctgacccttgaa

SEQ ID NO: 125 - DNA, 18 bp, Artificial Sequence
  synthetic oligonucleotide, USP11_ex5_F
gagcggtttctggtggag

SEQ ID NO: 126 - DNA, 26 bp, Artificial Sequence
  synthetic oligonucleotide, WIF1_ex9_F
gaacctgccatgaacccaacaaatgc

SEQ ID NO: 127 - DNA, 25 bp, Artificial Sequence
  synthetic oligonucleotide, WIF1_ex10_R
gccgctcctcggcctttttaagtga

SEQ ID NO: 128 - DNA, 25 bp, Artificial Sequence
  synthetic oligonucleotide, WIF1_ex9_nest_R
atggcaggttccatgtgcaccacag

SEQ ID NO: 129 - DNA, 18 bp, Artificial Sequence
  synthetic oligonucleotide, ZDHHC20_ex1_F
ctggagcgtccgagtcac

SEQ ID NO: 130 - DNA, 22 bp, Artificial Sequence
  synthetic oligonucleotide, ZDHHC20_ex2_R
caacggtctttccattttcttc

SEQ ID NO: 131 - DNA, 789 bp, Homo sapiens
cttcttgttgttaacagtttccggaaagagagtgatcgggatgcaagatttttgagactcactggtctgaaactgcctttcctcttaaatttagttaagtatttccctgggcatggaattcaaaggtggttatgacttttcttcaggattgtgaatgtatttatatcctcccatcttacgttgctattgagaattctgaagttcttctgattcctgattctttgtatgtgtattcctcattccacaccttccccagaatgcatgcagaatttcttcctttctattttcttttctctttttctgaaactcttattattgggatcttctgcctcttggtttagggctctaattttccgacattttctctgctattttttactactttatttttctcctctactttctgagagatttcctcctcttgatcttccaaatcttgtactgaatcttttatttttgttaacatgttcttaatttccaagaactcttttttcttgtcttcggagtttcaacacttattgttgttttgtgcatgtattattttttcttctctctctgaggctatttaggaaattttttattgaagctcctcccccctgcttccttcaagttgcttttatctgcttttttatttgctttttcatgcaataagtttttctcacatgtctggtaactcttggggattaccaaaaactcatagaaaattctgaccatgtgagtaacacttgccaattttgagcttcatgatagaatgatctagctggaccttttgctgcggggaaatcggag

SEQ ID NO: 132 - DNA, 777 bp, Homo sapiens
aacagtttccggaaagagagtgatcgggatgcaagatttttgagactcactggtctgaaactgcctttcctcttaaatttagttaagtatttccctgggcatggaattcaaaggtggttatgacttttcttcaggattgtgaatgtatttatatcctcccatcttacgttgctattgagaattctgaagttcttctgattcctgattctttgtatgtgtattcctcattccacaccttccccagaatgcatgcagaatttcttcctttctattttcttttctctttttctgaaactcttattattgggatcttctgcctcttggtttagggctctaattttccgacattttctctgctattttttactactttatttttctcctctactttctgagagatttcctcctcttgatcttccaaatcttgtactgaatcttttatttttgttaacatgttcttaatttccaagaactcttttttcttgtcttcggagtttcaacacttattgttgttttgtgcatgtattattttttcttctctctctgaggctatttaggaaattttttattgaagctcctcccccctgcttccttcaagttgcttttatctgcttttttatttgctttttcatgcaataagtttttctcacatgtctggtaactcttggggattaccaaaaactcatagaaaattctgaccatgtgagtaacacttgccaattttgagcttcatgatagaatgatctagctggaccttttgctgcggggaaatcggag

SEQ ID NO: 133 - DNA, 108 bp, Homo sapiens
tttccggaaagagagtgatcgggatgcaagatttttgagactcactggtctgaaactgcctttcctcttaaatttagttaagtatttccctgggcatggaattcaaag

SEQ ID NO: 134 - DNA, 149 bp, Homo sapiens
tgattactttccacctgcaaagtggaaacaataagaccagttagcggctgctgctttagtcaagcaagagatgaaggtgacttggaatatgctgtacagatggcattaaatgaatatggatctccttttggaaacttttcatctgcatg

SEQ ID NO: 135 - DNA, 185 bp, Homo sapiens
ttacttctatcccatcttaatacacttgcactgaagtgattactttccacctgcaaagtggaaacaataagaccagttagcggctgctgctttagtcaagcaagagatgaaggtgacttggaatatgctgtacagatggcattaaatgaatatggatctccttttggaaacttttcatctgcatg

(19) United States
(12) Patent Application Publication
(10) Pub. No.: US 2012/0172242 A1
(43) Pub. Date: Jul. 5, 2012

(54) CANCER SPECIFIC TRANSCRIPT VARIANTS

(75) Inventors: Rolf I. Skotheim, Oslo (NO); Guro E. Lind, Oslo (NO); Ragnhild A. Lothe, Oslo (NO); Marthe Eken, Oslo (NO); Gard O.S. Thomassen, Oslo (NO)

(73) Assignee: OSLO UNIVERSITETSSYKEHUS HF, Oslo (NO)

(21) Appl. No.: 13/139,033

(22) PCT Filed: Dec. 17, 2009
(86) PCT No.: PCT/IB09/07996

§ 371 Date: Aug. 15, 2011

(30) Foreign Application Priority Data
Dec. 17, 2008 (EP) 08172043.5

(51) Int. Cl. C40B 30/04; C12Q 1/68

(52) U.S. Cl. 506/9; 435/6.18; 435/6.14

(57) ABSTRACT

The present inventors here present a novel strategy for identification of RNA transcript variants and demonstrate that these can be correlated to disease states in mammals such as cancer. In particular, the transcript variants show prevalence and specificity to cancer, and thus also show clinical applicability in e.g. cancer diagnostics and prognostics, treatment and therapeutics. The present inventors have identified RNA transcript variants of VNN1 that can be used as biomarkers. The RNA transcript variant may also be used as biomarkers for diagnosing, prognosing, and/or monitoring a cancer.

### TECHNICAL FIELD OF THE INVENTION

The present invention relates to the identification of a new group of RNA transcript variants. In particular the present invention relates to RNA transcript variants comprising a 5′ and/or 3′ junction sequence(s) of a 5′ outlier exon, wherein said junction sequence(s) comprises an intron or extra-genic originating expressed sequence. An object of the present invention relates to a method for the detection of an abnormal gene expression of at least one RNA transcript variant of the gene VNN1. Another object of the present invention relates to the use of VNN1 RNA transcript variants as a biomarker. In particular the present invention relates to abnormal gene expressions in and biomarkers of cancer.

### BACKGROUND OF THE INVENTION

Alternative splicing of primary transcripts (pre-mRNAs), alternative promoter usage, and alternative polyadenylation sites are mechanisms giving rise to multiple mRNA transcript variants and subsequently multiple protein isoforms per gene, and adding additional dimensions to the cellular complexity. Alterations of these normal processes are common in cancer cells and result in the production of mRNAs not existing in healthy cells or in the modification of tissue-specific ratios between normal mRNA types. One explanation for these differences is the fundamental difference in expression patterns of known splicing-regulatory genes in cancerous as compared to normal tissues. Individual cancer-specific variants may or may not be functionally important for the cells, but nevertheless, and due to the presence of sequences only present in malignant cells, they have the potential to function as therapeutic targets or as biomarkers for cancer diagnostics and prognostics. This great potential makes discovery and characterisation of novel transcript variants an interesting path towards a better understanding and management of cancer.

Different alternative splicing mechanisms are known. Exons which are either skipped or included in the final mRNA and are flanked by intron sequences on both sides are called cassette exons. Another mechanism of alternative splicing is the use of different 5′ and 3′ splice sites, where the amount of sequence included from a particular exon varies between different transcripts. If a splice site is missed by the splicing machinery, an intron can be retained in the final mRNA and contribute to the coding sequence. Also, some exons are mutually exclusive. This means that in the final processed mRNA, one out of two exons is always present, but never both.

In addition, different splice variants from the same gene may have completely different activities, because whole functional domains may be added or deleted from the protein-coding sequence. An example of such alterations is seen in the anti-apoptotic gene BIRC5. This gene is highly upregulated in various cancers and alternative splicing of its pre-mRNA produces four different mRNAs, which encode four different protein isoforms. One isoform has pro-apoptotic properties and acts like a naturally occurring antagonist of the anti-apoptotic functions of the other isoforms.

When discussing alternative core promoter usage it is important to keep in mind the differences between a transcription start site (TSS) and a core promoter. A gene's TSS is the first nucleotide to be transcribed into a particular RNA. The core promoter, on the other hand, is the genomic region that surrounds a TSS. The length of a core promoter is defined as the segment of DNA required to recruit the transcription initiation complex and initiate transcription, given the appropriate external signals. Alternative TSSs are often used within a core promoter.

Use of alternative core promoters enables diversification of transcriptional regulation within a single gene and thereby plays a significant role in the control of gene expression in various cell lineages, tissue types and developmental stages. The use of different core promoters can lead to two types of protein products, depending on the location of the translational start site relative to the used promoter. If the translational start site exists within the first exon, mRNA isoforms that encode distinct proteins will be produced. On the other hand, if the alternative first exon is non-coding, the alternative transcripts will have heterogeneous 5′ untranslated regions (5′-UTR), which commonly implies different RNA stability, but the encoded proteins are identical. The molecular mechanisms behind the selective use of multiple promoters are not well known, but the use of diverse core promoter structures, variable concentrations of cis-regulatory elements and regional epigenetic mechanisms are thought to be important factors.

Several oncogenes and tumour suppressor genes have multiple promoters and the aberrant use of one promoter over another in some of these genes is directly linked to cancerous cell growth.

The most common method for genome-wide gene expression analysis is by use of DNA microarrays. Here, the expression levels of genes are measured by hybridisation signals to probes targeting predefined sequences. Thus, only exonic sequences known to the existing genome and transcriptome annotation are measured.

High-throughput sequencing of RNA (RNA-seq) is a powerful tool for identification of novel exons in individual samples. However, as of yet, a high cost make it unfeasible to process a large number of samples.

5′ rapid amplification of cDNA ends (5′-RACE) is a method to detect transcript sequences 5′ to a predefined gene-specific primer. In a large-scale effort to detect novel transcript structures, this method alone is in need of a good way to select candidate genes, the position of the RACE-primer, and the relevant samples to perform the RACE-experiments in.

Hence, an improved method for identification of novel RNA transcript variants would be advantageous, and in particular a more efficient and/or reliable method for identification of novel exons and exon-exon junction sequences in cancer samples would be advantageous.

The gene VNN1, encoding the vanin 1 protein, shares extensive sequence similarity with other members of the vanin gene family, which includes secreted and membrane-associated proteins. Detection of VNN1 expression was included in a blood-based biomarker panel for stratifying current risk for colorectal cancer (Marshall et al., Int. J. Cancer, 2009).

### SUMMARY OF THE INVENTION

Thus, an object of the present invention relates to a novel strategy for identification of transcript variants from a biological sample.

In particular, it is an object of the present invention to provide a method for identification of RNA transcript variants comprising a 5′ and/or 3′ junction sequence(s) of a 5′ outlier exon, wherein said junction sequence(s) comprises an intron or extra-genic originating expressed sequence in cancerous samples that solves the above mentioned problems of the prior art with regards to selection of candidate genes, selection of primer positions for RACE-PCR, and selection of the relevant samples with high likelihood of containing a novel transcript variant of the given candidate gene.

One aspect of the present invention relates to a method for the identification novel RNA transcript variant, by obtaining an exon expression profile of a gene in various test sample(s), obtaining a reference exon expression profile the gene in a reference sample, which may be taken from a control population such as a healthy population, identification of at least one 5′ outlier exon, identification of 5′ and/or 3′ junction sequence(s) of said 5′ outlier exon, and identification of RNA transcript variant comprising various parts of junction sequences.

Another aspect of the present invention relates to an RNA transcript variant comprising an 5′ and/or 3′ junction sequence(s) of an 5′ outlier exon, wherein said junction sequence(s) comprises an intron or extra-genic originating expressed sequence.

Yet another aspect of the present invention relates to method for the detection of an abnormal gene expression pattern by identifying the novel RNA transcript variant comprising an 5′ and/or 3′ junction sequence(s) of an 5′ outlier exon and comparing the expression level of such RNA transcript variant with a reference and correlating this to various diseases, such as cancer.

In conclusion, the present inventors here present a novel strategy for identification of these RNA transcript variants and furthermore demonstrate that these can be correlated to disease states in mammals. In particular the transcript variants show prevalence and specificity to cancer, and thus also show clinical applicability in e.g. cancer diagnostics, prognostics, treatment and therapeutics.

In addition, the present invention relates to a method for the detection of abnormal gene expression of VNN1 RNA transcript variants, said method comprising identifying an expression level of at least one RNA transcript variant of said at least one gene obtained from a test subject, comparing the expression level of said at least one RNA transcript variant of said at least one gene with a reference obtained from a reference subject, selecting a desired sensitivity, selecting a desired specificity, and indicating the test subject as likely to have abnormal gene expression, if the expression level of the said at least one RNA transcript variant of said at least one gene in the sample obtained from a test subject is significantly different from the reference, and indicating the test subject as unlikely to have abnormal gene expression, if the expression level of said at least one RNA transcript variant of said at least one gene is equal to the reference.

Another aspect of the present invention the abnormal expression pattern is indicative of cancer or a viral infection or a metabolic disease in the test subject.

Yet another aspect of the present invention relates to the use of at least one RNA transcript variant of VNN1 as a biomarker.

In an aspect of the present invention are these variants biomarkers for cancer.

Another aspect of the present invention relates to said biomarker as a biomarker for diagnosing, prognosing, and/or monitoring a cancer.

### BRIEF DESCRIPTION OF THE FIGURES

FIG. 1:

FIG. 1 shows representative nested RACE results from analysis of PRRX2, RAD51L1, and VNN1. Lanes one, two, and three shows the results from nested RACE for PRRX2, RAD51L1, and VNN1, respectively. Abbreviations: M1 500 base pair size marker; N1, negative control for PRRX1; N2, negative control for RAD51L1; N3, negative control for VNN1; M2, 100 base pair size marker.

FIG. 2:

FIG. 2 shows novel transcript variants of RAD51L1 in a colorectal cancer cell line. (A) Expression levels of the different probesets (often corresponding to the different exons) in RAD51L1 as seen from exon microarray data. Expression levels from the different cell lines are indicated by different shades and the thick lines represent the average for the six cell lines, ten colorectal carcinoma samples, and ten normal samples, respectively. The cell line SW48 deviates from the rest of the cell lines by showing stronger expression signals in the 3′-portion of the gene. (B) An overview of the different transcript variants. The black ruler on top indicates number of base pairs from the start of exon one. All exons are marked with a number. 1-14 indicates known exons or variants hereof, whereas α-η represents novel exons sequenced from SW48. The number of clones found with the same sequence is indicated in brackets after the name of the transcript. The start of every exon is in agreement with the number of base pairs from the start of exon one, but the exon width on the illustration is exaggerated for improved visualisation. Exons are numbered according to their location in the genomic sequence and exact positions of every exon can be found in Appendix II. Location of the nested gene-specific primer (NGSP) is shown by a black arrow. Five different transcripts are known according to Ensembl for RAD51L1. These transcripts have a total of 14 exons.

FIG. 3:

FIG. 3 shows results for NKAIN2. (A) Expression levels of the different exons in NKAIN2 for six cell lines. LS1034 has higher expression of exons eight to ten than the other cell lines. (B) Expression levels of the different exons in NKAIN2 for ten colorectal carcinomas. C1033III has higher expression of exons eight to ten than the other carcinomas. (C) An overview of the different transcript variants. Three different transcript variants are known for NKAIN2 according to Ensembl. Eight new transcripts were found by sequencing of the 5′-RACE products from LS1034 and C1033III and constitute a total of four new exons in introns four, eight, and nine. See legend of FIG. 2 for more detailed explanations.

FIG. 4:

FIG. 4 shows results for VNN1. (A) Expression levels of the different exons in VNN1 for six cell lines. HT29 deviates from the other cell lines by higher expression of exons six and seven. (B) An overview of the different transcript variants. One transcript with seven exons is known for VNN1. Three new transcript variants were found by sequencing of the 5′-RACE products from HT29 and include two new exons inside intron number five. See legend of FIG. 2 for more detailed explanations.

FIG. 5:

FIG. 5 shows results for C4BPB. (A) Expression levels of the different exons in C4BPB for ten colorectal carcinoma samples. C1034III deviates from the rest in exons two to eight. (B) An overview of the different transcript variants. Five transcripts with a total of seven exons are known for C4BPB. Three new transcript variants were found by sequencing of the 5′-RACE products. See legend of FIG. 2 for more detailed explanations.

FIG. 6:

FIG. 6 shows results for HOXC11. (A) Expression levels of the different exons in HOXC11 for ten colorectal carcinoma samples. One sample, C1402III, deviates from the rest in the end of exon one and all of exon two. (B) An overview of the different transcript variants. One transcript with two exons is known for HOXC11. Two new transcript variants were found by sequencing of the 5′-end of the cDNA. See legend of FIG. 2 for more detailed explanations.

FIG. 7:

FIG. 7 shows results for TFR2. (A) Expression levels for the different exons in TFR2 for six cell lines. Two cell lines, SW48 and RKO, deviate from the rest in exons eight to eighteen. (B) An overview of the different transcript variants. One transcript with eighteen exons is known for TFR2. Ten new transcript variants were found by sequencing of the 5′-end of the cDNA. See legend of FIG. 2 for more detailed explanations.

FIG. 8:

FIG. 8 shows results for SERPINB7. (A) Expression levels of the different exons in SERPINB7 for six cell lines. One cell line, LS1034, deviates from the rest in exons five to nine. (B) An overview of the different transcript variants. Two transcripts with a total of nine exons are known for SERPINB7. Three transcript variants were found by sequencing of the 5′-RACE products in LS1034. See legend of FIG. 2 for more detailed explanations.

FIG. 9:

FIG. 9 shows results for TFPT. (A) Expression levels of the different exons in TFPT for six cell lines. One cell line, SW48, deviates from the rest in exons four to seven. (B) An overview of the different transcript variants. Four different transcripts with seven exons are known for TFPT. Two transcript variants were found by sequencing of the 5′-RACE products from SW48. See legend of FIG. 2 for more detailed explanations.

FIG. 10:

FIG. 10 shows results for GJB6. (A) Expression levels of the different exons in GJB6 for six cell lines. One cell lines, HT29, deviates from the others by higher expression of exons five and six. (B) An overview of the different transcript variants. Four different transcripts with a total of six exons are known for GJB6. Six transcript variants were found by sequencing of the 5′-RACE products from HT29. See legend of FIG. 2 for more detailed explanations.

FIG. 11:

FIG. 11 shows results for PRRX1. (A) Expression levels of the different exons in PRRX1 for six cell lines. One cell line, SW48, deviates from the others by higher expression of exons two to five. (B) Overview of the different transcript variants. Two different transcripts with a total of five exons are known for PRRX1. Eight transcript variants were found by sequencing of the 5′-RACE products from SW48. See legend of FIG. 2 for more detailed explanations.

FIG. 12:

FIG. 12 shows results for PRRX2. (A) Expression levels of the different exons in PRRX2 for ten colorectal carcinoma samples. One sample, C1033III, deviates from the others by higher expression of exon number four. (B) An overview of the different transcript variants. One transcript with four exons is known for PRRX2 and two transcript variants were found by sequencing of the 5′-RACE products from C1033III. See legend of FIG. 2 for more detailed explanations.

FIG. 13: According to the latest version of Ensembl (release 56, September 2009), there is only one transcript annotated for VNN1. This variant, ENST00000367928, has seven exons. Three new transcript variants were found by sequencing of the 5′-RACE products from HT29 and include two new exons inside intron number five of ENST00000367928. To distinguish transcripts including the novel exons from the ENST00000367928 transcripts, an RT-PCR assay was developed with two specific forward primers and a common reverse primer. The forward primer targeting ENST00000367928 is specific to the annotated exon 5 and the forward primer targeting the novel transcripts target the common region in exon {alpha}.

FIG. 14: RT-PCR of VNN1 with primers specifically binding to the ENST00000367928 exon 5 to 6 from 8 normal colon mucosa (marked “N”), 105 colorectal cancers, 2 negative controls (marked “Neg”). PCR-product at the expected length was detected for all samples.

FIG. 15: RT-PCR of novel exons within VNN1 with one of the primers specifically binding to the novel exon {alpha} and the exon 6 in ENST00000367928 from 8 normal colon mucosa (marked “N”), 105 colorectal cancers, 2 negative controls (marked “Neg”). PCR-products at the expected lengths according to the 5′RACE experiments, and one additional band, were detected for 87% of the colorectal cancers, but not for any of the normal colon mucosa or the negative controls.

The present invention is described in more detail in the following.

### DETAILED DESCRIPTION OF THE INVENTION

The present invention provides methodology, which is employed in a screening strategy for the identification of transcript variants from a biological sample. The strategy includes the following objectives:
Investigate the expression level of individual exons in candidate genes, or in all genes in the genome, as from automated analyses of exon microarray data.
Investigate the 5′-end of mRNA from individual genes in cell lines and/or tumour samples where exon expression profile in the 3′-end of the gene that is different from that of a reference profile.

The strategy was established for candidate gene selection of genes with outlier expression profiles in colorectal cancer and for genes with known or putative involvement as oncogenic fusion transcripts. For all candidate genes, the expression levels across all exons were investigated, and genes with overexpression selectively from 3′-exons were further analysed for novel upstream sequences.

In total, the exon expression levels for 508 genes were investigated. Eleven of these genes had deviating exon expression profiles indicating qualitative changes in the transcript structure and were therefore further investigated. RNA transcript variants were identified in all of the eleven genes. These included potentially new promoters, novel exons within intron sequences and intron retentions, however, no fusion genes were found.

In conclusion, the present inventors here present methods for identification of RNA transcript variants and furthermore demonstrate that these can be correlated to disease states in mammals. In particular the transcript variants show prevalence and specificity to cancer, and thus also show clinical applicability in e.g. cancer diagnostics, prognostics, treatment and therapeutics.

Thus, one aspect of the present invention relates to a method for the identification of at least one RNA transcript variant, said method comprising obtaining an exon expression profile of a gene of interest in a test sample, obtaining a reference exon expression profile of said gene in a reference sample, identification of at least one 5′ outlier exon, identification of 5′ and/or 3′ junction sequence(s) of said 5′ outlier exon, and identification of at least one RNA transcript variant comprising at least one of said junction sequences.

### Exon Expression Profile

The exon expression profile as used herein refers to the individual expression measurements from two or more exons along a gene of interest. The expression profiles represent the abundance of the individual exons in the pool of RNA transcripts present in a sample. The expression measurements are reported as relative expression as compared to the corresponding exon expression profile of a reference. Such an exon expression profile is obtained from RNA or single/double-stranded cDNA. The profile can be obtained as an average expression from 1 to ˜n number of samples.

Analysis of the exon expression profile turned to be an important step in the process of enriching for genes with alterations in their transcript structures. If every exon in a gene is under the control of the same promoter, it is expected that the exon expression levels to be similar throughout the gene.

If, on the other hand, a gene has a second alternative promoter, the exons downstream of the new promoter/breakpoint will be under the control of a different promoter than the upstream exons. The 5′-portion of the original gene is therefore regulated by one promoter and the 3′-portion by another, leading to different expression of the two parts.

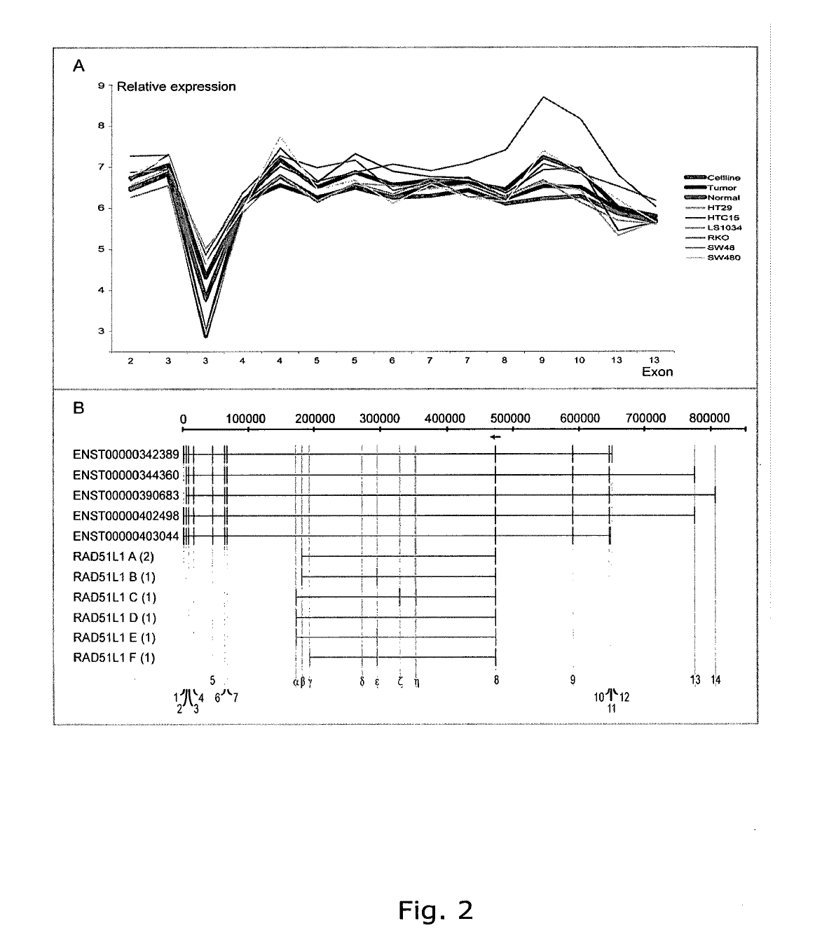

This may give rise to longitudinal exon expression profiles looking like the ones seen in FIG. 2A to FIG. 12A, where exons in the 3′-end of a gene have higher expression than the 5′-exons in certain samples as compared to others.

Thus, an expression profile of a sample as compared to that of a reference can be compared statistically.

The statistical significance may be determined by the standard statistical methodology known by the person skilled in the art.

### Outlier Transcript Profile

An outlier transcript profile refers to a transcript profile, where the relative exon expression profile of the test sample vs. the reference sample is higher in the 3′-portion of the transcript (one or more exons at the 3′-end) as compared to the 5′-end of the transcript (one or more exons at the 5′-end) with statistical significance.

An embodiment of the present invention refers to a an outliner transcript profile, wherein the relative profile of the test sample vs. the reference sample is significantly higher in the 3′-portion of the transcript (one or more exons at the 3′-end) as compared to the 5′-end of the transcript (one or more exons at the 5′-end) with a confidence interval of 50%, such as 75%, such as 90%, such as 95%, such as 99%.

The significance may be determined by the standard statistical methodology known by the person skilled in the art.

### Identification of at Least One 5′ Outlier Exon

Identification of at least one 5′ outlier exon as used herein refers to the identification of at least the first 5′ outlier exon in an exon expression profile. One method for identification of an exon expression profile indicating the existence of such 5′ exon can be through calculation of two probabilities for each exon-exon junction. A first probability is based on a t-test for whether values from all upstream and all downstream exons are likely to belong to different populations [P(transcript)]. A second probability is based on a t-test for whether the values from the immediate up- and downstream exons are likely to belong to different populations [P(exon)]. A Transcript breakpoint score (TBS) is calculated as the product of the two [TBS=P(transcript)*P(exon)].

In statistics is a confidence interval (CI) or confidence bound is an interval estimate of a population parameter. Instead of estimating the parameter by a single value, an interval likely to include the parameter is given. Thus, confidence intervals are used to indicate the reliability of an estimate. How likely the interval is to contain the parameter is determined by the confidence level or confidence coefficient. Increasing the desired confidence level will widen the confidence interval. For example, a CI can be used to describe how reliable survey results are. A 95% confidence interval for the proportion in the whole population having the same intention on the survey date might be 36% to 44%. All other things being equal, a survey result with a small CI is more reliable than a result with a large CI and one of the main things controlling this width in the case of population surveys is the size of the sample questioned. Confidence intervals and interval estimates more generally have applications across the whole range of quantitative studies.

If a statistic is presented with a confidence interval, and is claimed to be statistically significant, the underlying test leading to that claim will have been performed at a significance level of 100% minus the confidence level of the interval.

Accordingly an embodiment of the present invention refers to a method for identification of a 5′ outlier exon of the invention that can be indentified through calculation of two probabilities for each exon-exon junction. One probability is based on a t-test for whether values from all upstream and all downstream exons are likely to belong to different populations [P(transcript)]. A second probability is based on a t-test for whether the values from the immediate up- and downstream exons are likely to belong to different populations [P(exon)]. A Transcript breakpoint score (TBS) is calculated as the product of the two [TBS=P(transcript)*P(exon)] with a confidence interval of 50%, such as 75%, such as 90%, such as 95%, such as 99%.

### Intron or Extra-Genic Originating Expressed Transcripts

Intron or extra-genic originating expressed sequence also referred to as intergenic sequences as used herein refers to novel transcript sequences that have previously been annotated as intronic or intergenic or a sequence that have not been annotated before. That is, Ensembl and RefSeq do not consider these sequences as part of the reference transcripts of the human genome.

### Expressed Transcript

An expressed transcript as used herein refers to a transcript that is encoded by a gene and expressed to form a transcript RNA. This RNA can be coding, or non-coding.

### Junction Sequence

A junction according to the present invention refers to the intersection of genetic elements such as exons and introns. Accordingly, the junction sequence refers to the sequence spanning the flanking sequence of the junction. Thus, the junction sequence of two juxtaposing exons in a mRNA comprises the 3′ flanking sequence of the 5′ exon and the 5′ flanking sequence of the 3′ exon.

Hence, the 5′ junction sequence of a particular exon will contain at least part of the 5′ end of the exon of interest and at least part of the 3′ flanking sequence of the 5′ exon. Similarly will the 3′ junction sequence of an exon contain at least part of the 3′ end of the exon of interest and at least part of 5′ flanking sequence of the 5′ exon.

In an embodiment 5′ and/or the 3′ junction sequences of the present invention are identified by sequencing of a polynucleotide obtained from RACE, one-sided PCR and/or anchored PCR.

In one embodiment the 5′ flanking sequence is less than 15 kb, such as less than 10 kb, for example less than such as 10 kb, for example less than such as 5 kb, for example less than such as 4 kb, for example less than such as 3 kb, for example less than such as 2 kb, for example less than such as 1 kb, for example less than such as 500b.

In one embodiment the 3′ flanking sequence is less than 15 kb, such as less than 10 kb, for example less than such as 10 kb, for example less than such as 5 kb, for example less than such as 4 kb, for example less than such as 3 kb, for example less than such as 2 kb, for example less than such as 1 kb, for example less than such as 500b.

### RNA Transcript Variant

An aspect of the present invention relates to an RNA transcript variant comprising an 5′ and/or 3′ junction sequence(s) of an 5′ outlier exon, wherein said junction sequence(s) comprises an intron or extra-genic originating expressed sequence.

Another aspect of the present invention relates to an isolated RNA transcript variant obtained from a method for the identification of at least one RNA transcript variant, said method comprising obtaining an exon expression profile of a gene of interest in a test sample, obtaining a reference exon expression profile of said gene in a reference sample, identification of at least one 5′ outlier exon, identification of 5′ and/or 3′ junction sequence(s) of said 5′ outlier exon, and identification of at least one RNA transcript variant comprising at least one of said junction sequences.

A transcription start site TSS of a gene is the first nucleotide to be transcribed into a particular RNA. The core promoter, on the other hand, is the genomic region that surrounds a TSS. The length of a core promoter is defined as the segment of DNA required to recruit the transcription initiation complex and initiate transcription, given the appropriate external signals. Alternative TSSs are often used within a core promoter. Thus, the RNA transcripts, which are products of transcriptional initiation from different TTSs, will have different terminal 5′ flanking sequences.

In one embodiment the RNA transcript variant is the transcriptional product of a core promoter. The core promoter may be activated by various stimuli and the aberrant core promoter activity may correlate with clinical conditions such as cancer, viral infections and metabolic conditions.

A 5′ cap structure is found on the 5′ end of an mRNA molecule and consists of a 7-methylguanosine connected to the mRNA via a 5′ to 5′ triphosphate linkage.

In one embodiment the junction is the 5′ to 5′ triphosphate bridge linking the 7-methylguanosine to 5′ end of the RNA transcript variant. Accordingly, in one particular embodiment the junction sequences is the 5′ flanking sequences of the 5′ outlier exon and 7-methylguanosine linked by the 5′ to 5′ triphosphate bridge. This structure is the 5′ capture and the 5′ terminal sequences of the 5′ outlier exon, which identifies the RNA transcript variant of the embodiment.

RNA transcript variant as used herein refers to any RNAs that comprises exons, introns or part hereof originating from the same gene. The RNA transcript variant can arise through alternative or aberrant pre-mRNA processing, alternative or aberrant promoter usage or polyadenylation initiation sites.

This means that one or more exons or exon-junctions are differentially included in the RNA transcript variants of a particular gene. Thus, can the RNA transcript variants be one exon, two exons, three exons, or more exons of a particular gene.

RNA transcript variants can result in polypeptides, but can also be non-coding.

### Expression Level

The expression level of a given genetic element as used herein refers to the absolute or relative amount of RNA corresponding to this genetic element in a given sample. Expressed genes include genes that are transcribed into mRNA and then translated into protein, as well as genes that are transcribed into mRNA, or other types of RNA such as, tRNA, rRNA or other non-coding RNAs, that are not translated into protein. RNA expression is a highly specific process which can be monitored by detecting the absolute or relative RNA levels.

Thus, the expression level refers to the amount of RNA in a sample. The expression level is usually detected using microarrays, northern blotting, RT-PCR, SAGE, RNA-seq, or similar RNA detection methods.

When expression levels of a specific RNA in a test sample is compared to a reference sample they can either be different or equal. However, using today's detection techniques is an exact definition of different or equal result can be difficult because of noise and variations in obtained expression levels from different samples. Hence, the usual method for evaluating whether two or more expression levels are different or equal involves statistics.

Statistics enables evaluation of significantly different expression levels and significantly equal expressions levels. Statistical methods involve applying a function/statistical algorithm to a set of data. Statistical theory defines a statistic as a function of a sample where the function itself is independent of the sample's distribution: the term is used both for the function and for the value of the function on a given sample. Commonly used statistical tests or methods applied to a data set include t-test, f-test or even more advanced test and methods of comparing data. Using such a test or methods enables a conclusion of whether two or more samples are significantly different or significantly equal.

### Abnormal Gene Expression Pattern

The expression of a gene results in at least one RNA transcript. As used herein an abnormal gene expression pattern refers to a significantly different expression level of a gene in a test sample as compared to a reference sample.

An embodiment of the present invention refers to an abnormal gene expression pattern refers to a significantly different expression level of a gene in a test sample as compared to a reference sample with a confidence interval of 50%, such as 75%, such as 90%, such as 95%, such as 99%.

Accordingly, one embodiment relates to a method for the identification of at least one RNA transcript variant, wherein the expression of the 5′ outlier exon is significantly higher than the corresponding 5′ exon of the reference.

Accordingly, one embodiment relates to a method for the identification of at least one RNA transcript variant, wherein the expression of the 5′ outlier exon is significantly lower than the corresponding 5′ exon of the reference.

In a further embodiment the expression level of each of the 3′ exons from said test sample are higher than their corresponding 3′ exons of the reference.

The significance may be determined by the standard statistical methodology known by the person skilled in the art.

### Correlation of Abnormal Gene Expression to a Disease State

Another aspect of the invention relates to method for the detection of an abnormal gene expression pattern, said method comprising identifying an expression level of an RNA transcript variant comprising an 5′ and/or 3′ junction sequence(s) of an 5′ outlier exon, wherein said junction sequence(s) comprises an intron or extra-genic originating expressed sequence in a sample obtained from a test subject, comparing the expression level of said RNA transcript variant with a reference obtained from a reference subject, selecting a desired sensitivity, selecting a desired specificity, and indicating the test subject as likely to have an abnormal gene expression pattern, if the expression level of the RNA transcript variant in the sample obtained from a test subject is significantly different from the reference, and indicating the test subject as unlikely to have an abnormal gene expression pattern, if the expression level of the RNA transcript variant is equal to the reference.

Another aspect of the present invention relates to a method for the detection of an abnormal gene expression of at least one gene, wherein said at least one gene is VNN1, said method comprising identifying an expression level of at least one RNA transcript variant of said at least one gene in a sample obtained from a test subject, comparing the expression level of said at least one RNA transcript variant of said at least one gene with a reference obtained from a reference subject, selecting a desired sensitivity, selecting a desired specificity, indicating the test subject as likely to have abnormal gene expression, if the expression level of said at least one RNA transcript variant of said at least one gene in the sample obtained from a test subject is significantly different from the reference, and indicating the test subject as unlikely to have abnormal gene expression, if the expression level of said at least one RNA transcript variant of said at least one gene is equal to the reference.

In an embodiment relates to the method for the detection of an abnormal gene expression of at least one gene, such as one gene, such as two genes, such as three genes, such as four genes, such as five genes.

Yet another aspect of the present invention relates to a method for the detection of abnormal gene expression of at least one gene, wherein said at least one gene is VNN1, said method comprising the step of determining an expression level of at least one RNA transcript variant of said at least one gene in a sample obtained from a test subject.

Another aspect of the present invention relates to a method for the detection of abnormal gene expression of at least one gene, wherein said at least one gene is VNN1, said method comprising the step of determining an expression level of at least one RNA transcript variant of said at least one gene in a sample obtained from a test subject further comprising the steps of comparing the expression level of said at least one RNA transcript variant of said at least one gene with a reference obtained from a reference subject, selecting a desired sensitivity, selecting a desired specificity, indicating the test subject as likely to have an abnormal gene expression, if the expression level of said at least one RNA transcript variant of said at least one gene in the sample obtained from a test subject is significantly different from the reference, and indicating the test subject as unlikely to have an abnormal gene expression, if the expression level of said at least one RNA transcript variant of said at least one gene is equal to the reference.

In an embodiment of the present invention is the expression level of the at least one RNA transcript variant of the gene in the test subject higher than the reference subject.

In another embodiment of the present invention is the RNA transcript variant selected from the group consisting of VNN1 A (SEQ ID NO: 15), VNN1 B (SEQ ID NO: 16), and VNN1 C (SEQ ID NO: 17).

In another embodiment of the present invention comprises the RNA transcript variant one or more of the exons selected from the group consisting of VNN1α (SEQ ID NO:131), VNN1α′ (SEQ ID NO:132), VNN1α″ (SEQ ID N0133), VNN1β (SEQ ID NO:134), and VNN1β′ (SEQ ID NO:135).

The appearance or increase of a RNA transcript variant in a cell such as a neoplastic cell for example a tumour cell indicates a phenotypic change of the cells present in a sample obtained from said subject compared to a the corresponding cells in a sample from a reference subject.

The appearance or increase of a RNA transcript variant in cells of a sample obtained from neoplastic tissue for example a tumour tissue may therefore be indicative of a gain-of-function of an oncogene involved in the progression of carcinogenesis of the tumour. Accordingly, the RNA transcript variant is a potential candidate biomarker applicable for the diagnosis of the diseased state i.e. cancer.

In additionally embodiment can the RNA transcript variant be used as a biomarker for the progression of the disease state by monitoring of differential expression patterns over time.

Accordingly will the RNA transcript variant be applicable for diagnosis, prognosis and a treatment of clinical conditions or a diseased state.

Thus, in an embodiment is the expression level of an RNA transcript variant in the test subject is significantly higher or lower than the reference subject.

In another embodiment is the expression level of each of the 3′ exons from said test sample higher than their corresponding 3′ exons of the reference.

The significance may be determined by the standard statistical methodology known by the person skilled in the art.

In an embodiment the expression level of an RNA transcript variant is applicable for the diagnosis of a diseased state i.e. cancer, a viral infection or a metabolic disease in the test subject.

In another embodiment of the present invention, the abnormal expression pattern is indicative of cancer or an inflammatory disease or a viral infection or a metabolic disease in the test subject.

In a specific embodiment of the present invention, the cancer is selected from the group consisting of colorectal cancer, prostate cancer, breast cancer, lung cancer, liver cancer, kidney cancer, ovarian cancer, endometrial cancer, pancreatic cancer, brain cancer, testicular cancer, leukemia, lymphoma, sarcoma.

In a specific embodiment of the present invention is the cancer is colorectal cancer or the precursor to cancer is colorectal adenomas.

In an aspect of the present invention relates to the genomic genes that incode the RNA transcript variants of the present invention. The RNA transcript variants can be detected in the genomic DNA using standard DNA assaying techniques that are known in the art.

Thus relates one anspect of the present invention to detection and/or correlation of the genomic DNA encoding the RNA transcript variants of the present invention with cancer or an inflammatory disease or a viral infection or a metabolic disease in the test subject.

One embodiment of the present invention relates to an isolated nucleic acid molecule selected from the group consisting of SEQ ID NO 1, SEQ ID NO 2, SEQ ID NO 3, SEQ ID NO 4, SEQ ID NO 5, SEQ ID NO 6, SEQ ID NO 7, SEQ ID NO 8, SEQ ID NO 9, SEQ ID NO 10, SEQ ID NO 11, SEQ ID NO 12, SEQ ID NO 13, SEQ ID NO 14, SEQ ID NO 15, SEQ ID NO 16, SEQ ID NO 17, SEQ ID NO 18, SEQ ID NO 19, SEQ ID NO 20, SEQ ID NO 21, SEQ ID NO 22, SEQ ID NO 23, SEQ ID NO 24, SEQ ID NO 25, SEQ ID NO 26, SEQ ID NO 27, SEQ ID NO 28, SEQ ID NO 29, SEQ ID NO 30, SEQ ID NO 31, SEQ ID NO 32, SEQ ID NO 33, SEQ ID NO 34, SEQ ID NO 35, SEQ ID NO 36, SEQ ID NO 37, SEQ ID NO 38, SEQ ID NO 39, SEQ ID NO 40, SEQ ID NO 41, SEQ ID NO 42, SEQ ID NO 43, SEQ ID NO 44, SEQ ID NO 45, SEQ ID NO 46, SEQ ID NO 47, SEQ ID NO 48, SEQ ID NO 49, SEQ ID NO 50, SEQ ID NO 51, SEQ ID NO 52, SEQ ID NO 53, SEQ ID NO 54, SEQ ID NO 131, SEQ ID NO 132, SEQ ID NO 133, SEQ ID NO 134, and SEQ ID NO 135, that can be correlated to an abnormal gene expression pattern.

The above sequences are identified using the methodology of the present invention described herein. Thus, these sequences represent RNA transcript variants that are present and/or expressed to a higher level than the reference sample.

Accordingly, an embodiment of the invention relates to a biomarker selected from the group consisting of SEQ ID NO 1, SEQ ID NO 2, SEQ ID NO 3, SEQ ID NO 4, SEQ ID NO 5, SEQ ID NO 6, SEQ ID NO 7, SEQ ID NO 8, SEQ ID NO 9, SEQ ID NO 10, SEQ ID NO 11, SEQ ID NO 12, SEQ ID NO 13, SEQ ID NO 14, SEQ ID NO 15, SEQ ID NO 16, SEQ ID NO 17, SEQ ID NO 18, SEQ ID NO 19, SEQ ID NO 20, SEQ ID NO 21, SEQ ID NO 22, SEQ ID NO 23, SEQ ID NO 24, SEQ ID NO 25, SEQ ID NO 26, SEQ ID NO 27, SEQ ID NO 28, SEQ ID NO 29, SEQ ID NO 30, SEQ ID NO 31, SEQ ID NO 32, SEQ ID NO 33, SEQ ID NO 34, SEQ ID NO 35, SEQ ID NO 36, SEQ ID NO 37, SEQ ID NO 38, SEQ ID NO 39, SEQ ID NO 40, SEQ ID NO 41, SEQ ID NO 42, SEQ ID NO 43, SEQ ID NO 44, SEQ ID NO 45, SEQ ID NO 46, SEQ ID NO 47, SEQ ID NO 48, SEQ ID NO 49, SEQ ID NO 50, SEQ ID NO 51, SEQ ID NO 52, SEQ ID NO 53, SEQ ID NO 54, SEQ ID NO 131, SEQ ID NO 132, SEQ ID NO 133, SEQ ID NO 134, and SEQ ID NO 135.

A biomarker can be a marker for a diseased state i.e. cancer, a viral infection, a metabolic disease or an inflammatory disease in the test subject.

In another embodiment of the present invention, the biomarker is indicative of cancer or a viral infection or a metabolic disease in the test subject.

In a specific embodiment of the present invention, the cancer is selected from group consisting of colorectal cancer, prostate cancer, breast cancer, lung cancer, liver cancer, kidney cancer, ovarian cancer, endometrial cancer, pancreatic cancer, brain cancer, testicular cancer, leukemia, lymphoma, sarcoma.

An aspect of the present invention relates to the use of at least one RNA transcript variant selected from the list consisting of (SEQ ID NO:15), (SEQ ID NO:16), (SEQ ID NO:17), (SEQ ID NO:18), (SEQ ID NO:131), (SEQ ID NO:132), (SEQ ID NO:133), (SEQ ID NO:134), and (SEQ ID NO:135) as a biomarker.

Another aspect of the present invention relates to the use of the biomarker as a biomarker for diagnosing, prognosing, and/or monitoring a cancer.

Another aspect of the present invention relates to the use of the biomarker as a biomarker for diagnosing, prognosing, and/or monitoring a cancer, wherein the cancer is selected from group consisting of colorectal cancer, prostate cancer, breast cancer, lung cancer, liver cancer, kidney cancer, ovarian cancer, endometrial cancer, pancreatic cancer, brain cancer, testicular cancer, leukemia, lymphoma, sarcoma.

A further embodiment of the invention relates to an isolated nucleic acid molecule selected from the group consisting of SEQ ID NO 1, SEQ ID NO 2, SEQ ID NO 3, SEQ ID NO 4, SEQ ID NO 5, SEQ ID NO 6, SEQ ID NO 7, SEQ ID NO 8, SEQ ID NO 9, SEQ ID NO 10, SEQ ID NO 11, SEQ ID NO 12, SEQ ID NO 13, SEQ ID NO 14, SEQ ID NO 15, SEQ ID NO 16, SEQ ID NO 17, SEQ ID NO 18, SEQ ID NO 19, SEQ ID NO 20, SEQ ID NO 21, SEQ ID NO 22, SEQ ID NO 23, SEQ ID NO 24, SEQ ID NO 25, SEQ ID NO 26, SEQ ID NO 27, SEQ ID NO 28, SEQ ID NO 29, SEQ ID NO 30, SEQ ID NO 31, SEQ ID NO 32, SEQ ID NO 33, SEQ ID NO 34, SEQ ID NO 35, SEQ ID NO 36, SEQ ID NO 37, SEQ ID NO 38, SEQ ID NO 39, SEQ ID NO 40, SEQ ID NO 41, SEQ ID NO 42, SEQ ID NO 43, SEQ ID NO 44, SEQ ID NO 45, SEQ ID NO 46, SEQ ID NO 47, SEQ ID NO 48, SEQ ID NO 49, SEQ ID NO 50, SEQ ID NO 51, SEQ ID NO 52, SEQ ID NO 53, SEQ ID NO 54, SEQ ID NO 131, SEQ ID NO 132, SEQ ID NO 133, SEQ ID NO 134, and SEQ ID NO 135, which encodes a polypeptide.

An embodiment of the present invention relates to antibodies raised against the polypeptides of the present invention and use hereof for therapeutic purposes.

A further embodiment the invention relates to an isolated nucleic acid molecule selected from the group consisting of SEQ ID NO 1, SEQ ID NO 2, SEQ ID NO 3, SEQ ID NO 4, SEQ ID NO 5, SEQ ID NO 6, SEQ ID NO 7, SEQ ID NO 8, SEQ ID NO 9, SEQ ID NO 10, SEQ ID NO 11, SEQ ID NO 12, SEQ ID NO 13, SEQ ID NO 14, SEQ ID NO 15, SEQ ID NO 16, SEQ ID NO 17, SEQ ID NO 18, SEQ ID NO 19, SEQ ID NO 20, SEQ ID NO 21, SEQ ID NO 22, SEQ ID NO 23, SEQ ID NO 24, SEQ ID NO 25, SEQ ID NO 26, SEQ ID NO 27, SEQ ID NO 28, SEQ ID NO 29, SEQ ID NO 30, SEQ ID NO 31, SEQ ID NO 32, SEQ ID NO 33, SEQ ID NO 34, SEQ ID NO 35, SEQ ID NO 36, SEQ ID NO 37, SEQ ID NO 38, SEQ ID NO 39, SEQ ID NO 40, SEQ ID NO 41, SEQ ID NO 42, SEQ ID NO 43, SEQ ID NO 44, SEQ ID NO 45, SEQ ID NO 46, SEQ ID NO 47, SEQ ID NO 48, SEQ ID NO 49, SEQ ID NO 50, SEQ ID NO 51, SEQ ID NO 52, SEQ ID NO 53, SEQ ID NO 54, SEQ ID NO 131, SEQ ID NO 132, SEQ ID NO 133, SEQ ID NO 134, and SEQ ID NO 135, which is a non-coding RNA.

In another embodiment the non-coding RNA is selected from the group consisting of pre-miRNA, pri-miRNA, miRNA, snRNA.

In another embodiment, the isolated nucleic acid comprises a sequence sharing at least 90% identity with that set forth in the group consisting of SEQ ID NO 1, SEQ ID NO 2, SEQ ID NO 3, SEQ ID NO 4, SEQ ID NO 5, SEQ ID NO 6, SEQ ID NO 7, SEQ ID NO 8, SEQ ID NO 9, SEQ ID NO 10, SEQ ID NO 11, SEQ ID NO 12, SEQ ID NO 13, SEQ ID NO 14, SEQ ID NO 15, SEQ ID NO 16, SEQ ID NO 17, SEQ ID NO 18, SEQ ID NO 19, SEQ ID NO 20, SEQ ID NO 21, SEQ ID NO 22, SEQ ID NO 23, SEQ ID NO 24, SEQ ID NO 25, SEQ ID NO 26, SEQ ID NO 27, SEQ ID NO 28, SEQ ID NO 29, SEQ ID NO 30, SEQ ID NO 31, SEQ ID NO 32, SEQ ID NO 33, SEQ ID NO 34, SEQ ID NO 35, SEQ ID NO 36, SEQ ID NO 37, SEQ ID NO 38, SEQ ID NO 39, SEQ ID NO 40, SEQ ID NO 41, SEQ ID NO 42, SEQ ID NO 43, SEQ ID NO 44, SEQ ID NO 45, SEQ ID NO 46, SEQ ID NO 47, SEQ ID NO 48, SEQ ID NO 49, SEQ ID NO 50, SEQ ID NO 51, SEQ ID NO 52, SEQ ID NO 53, SEQ ID NO 54, SEQ ID NO 131, SEQ ID NO 132, SEQ ID NO 133, SEQ ID NO 134, and SEQ ID NO 135, such as 90% identity, 91% identity, 92% identity, 93% identity, 94% identity, 95 identity, 96% identity, 97% identity, 98% identity, or 99% identity.

### Sequence Identity

As commonly defined “identity” is here defined as sequence identity between genes or proteins at the nucleotide or amino acid level, respectively.

Thus, in the present context “sequence identity” is a measure of identity between proteins at the amino acid level and a measure of identity between nucleic acids at nucleotide level. The protein sequence identity may be determined by comparing the amino acid sequence in a given position in each sequence when the sequences are aligned. Similarly, the nucleic acid sequence identity may be determined by comparing the nucleotide sequence in a given position in each sequence when the sequences are aligned.

To determine the percent identity of two nucleic acid sequences or of two amino acids, the sequences are aligned for optimal comparison purposes (e.g., gaps may be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment the two sequences are the same length.

One may manually align the sequences and count the number of identical nucleic acids or amino acids. Alternatively, alignment of two sequences for the determination of percent identity may be accomplished using a mathematical algorithm. Such an algorithm is incorporated into the NBLAST and XBLAST programs of (Altschul et al. 1990). BLAST nucleotide searches may be performed with the NBLAST program, score=100, wordlength=12, to obtain nucleotide sequences homologous to a nucleic acid molecules of the invention. BLAST protein searches may be performed with the XBLAST program, score=50, wordlength=3 to obtain amino acid sequences homologous to a protein molecule of the invention. To obtain gapped alignments for comparison purposes, Gapped BLAST may be utilised. Alternatively, PSI-Blast may be used to perform an iterated search which detects distant relationships between molecules. When utilising the NBLAST, XBLAST, and Gapped BLAST programs, the default parameters of the respective programs may be used. See http://www.ncbi.nlm.nih.gov. Alternatively, sequence identity may be calculated after the sequences have been aligned e.g. by the BLAST program in the EMBL database (www.ncbi.nlm.gov/cgi-bin/BLAST). Generally, the default settings with respect to e.g. “scoring matrix” and “gap penalty” may be used for alignment. In the context of the present invention, the BLASTN and PSI BLAST default settings may be advantageous.

The percent identity between two sequences may be determined using techniques similar to those described above, with or without allowing gaps. In calculating percent identity, only exact matches are counted.

### Sensitivity

As used herein the sensitivity refers to the measures of the proportion of actual positives which are correctly identified as such—in analogy with a diagnostic test, i.e. the percentage of sick people who are identified as having the condition.

Usually the sensitivity of a test can be described as the proportion of true positives of the total number with the target disorder. All patients with the target disorder are the sum of (detected) true positives (TP) and (undetected) false negatives (FN).

### Specificity

As used herein the specificity refers to measures of the proportion of negatives which are correctly identified—i.e. the percentage of well people who are identified as not having the condition. The ideal diagnostic test is a test that has 100% specificity, i.e. only detects diseased individuals and therefore no false positive results, and 100% sensitivity, i.e. detects all diseased individuals and therefore no false negative results.

For any test, there is usually a trade-off between each measure. For example in a manufacturing setting in which one is testing for faults, one may be willing to risk discarding functioning components (low specificity), in order to increase the chance of identifying nearly all faulty components (high sensitivity). This trade-off can be represented graphically using a ROC curve.

Selecting a sensitivity and specificity it is possible to obtain the optimal outcome in a detection method. In determining the discriminating value distinguishing subjects or individuals having or developing e.g. colorectal cancer, the person skilled in the art has to predetermine the level of specificity. The ideal diagnostic test is a test that has 100% specificity, i.e. only detects diseased individuals and therefore no false positive results, and 100% sensitivity, i.e. detects all diseased individuals and therefore no false negative results. However, due to biological diversity no method can be expected to have 100% sensitive without including a substantial number of false negative results.

The chosen specificity determines the percentage of false positive cases that can be accepted in a given study/population and by a given institution. By decreasing specificity an increase in sensitivity is achieved. One example is a specificity of 95% which will result in a 5% rate of false positive cases. With a given prevalence of 1% of e.g. colorectal cancer in a screening population, a 95% specificity means that 5 individuals will undergo further physical examination in order to detect one (1) cancer case if the sensitivity of the test is 100%.

The cut-off level could be established using a number of methods, including: percentiles, mean plus or minus standard deviation(s); multiples of median value; patient specific risk or other methods known to those who are skilled in the art.

### Sample

In the present context, the term “sample” relates to any liquid or solid sample collected from an individual to be analyzed. Preferably, the sample is liquefied at the time of assaying.

In another embodiment of the present invention, a minimum of handling steps of the sample is necessary before measuring the expression of a RNA/cDNA. In the present context, the subject “handling steps” relates to any kind of pre-treatment of the liquid sample before or after it has been applied to the assay, kit or method. Pre-treatment procedures includes separation, filtration, dilution, distillation, concentration, inactivation of interfering compounds, centrifugation, heating, fixation, addition of reagents, or chemical treatment.

In accordance with the present invention, the sample to be analyzed is collected from any kind of mammal, including a human being, a pet animal, a zoo animal and a farm animal.

In yet another embodiment of the present invention, the sample is derived from any source such as body fluids.

Preferably, this source is selected from the group consisting of milk, semen, blood, serum, plasma, saliva, faeces, urine, sweat, ocular lens fluid, cerebral spinal fluid, cerebrospinal fluid, ascites fluid, mucous fluid, synovial fluid, peritoneal fluid, vaginal discharge, vaginal secretion, cervical discharge, cervical or vaginal swab material or pleural, amniotic fluid and other secreted fluids, substances, cultured cells, and tissue biopsies from organs such as the brain, heart and intestine.

One embodiment of the present invention relates to a method according to the present invention, wherein said body sample or biological sample is selected from the group consisting of blood, faeces, urine, pleural fluid, oral washings, vaginal washings, cervical washings, cultured cells, tissue biopsies, and follicular fluid.

Another embodiment of the present invention relates to a method according to the present invention, wherein said biological sample is selected from the group consisting of blood, plasma and serum.

In a presently preferred embodiment of the present invention relates to a method according to the present invention, wherein said biological sample is serum.

The sample taken may be dried for transport and future analysis. Thus the method of the present invention includes the analysis of both liquid and dried samples.

### Test Sample

The test sample as used herein refers to a RNA/cDNA sample, and can be of any source.

### Reference

As used herein can a reference refer to a reference sample or a reference subject.

### Reference Sample

The reference sample can consist of one or more RNA/cDNA samples, and can be of any source.

In some embodiments is the reference another gene or an intragenetic reference such as an exon within the gene and/or RNA transcript variant of interest.

In an embodiment of the present invention is the expression of one or more specific exons in the RNA transcript variants used as reference.

In a more specific embodiment of the present invention are these specific exons in the RNA transcript variants exon 1, exon 2, exon 3, exon 4′, exons, exon 6, exon 7 for VNN1.

The genetic boundaries of the exons can be found in the examples and tables of the present application.

In some embodiments the reference sample is from the same species as the comparable test sample. The reference sample can be obtained as an average expression from 1 to ˜n number of samples. The reference sample can also reflect a pool of reference samples.

### Test Subject

As used herein refers a test subject to the subject from which the test sample is obtained.

In accordance with one embodiment of the present invention, the sample to be analyzed may be collected from any kind of mammal, including a human being, a pet animal, a zoo animal and a farm animal.

### Reference Subject

As used herein a reference subject refers to the mammal from which the reference sample is obtained.

The reference subject can be obtained as an average from 1 to ˜n number of subjects or seen as a population.

In accordance with the present invention, the sample to be analyzed is collected from any kind of mammal, including a human being, a pet animal, a zoo animal and a farm animal.

### General

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Materials and Methods

### Colorectal Cell Lines and Tissue Samples.

The project involved analyses of six colon carcinoma cell lines (HT29, HCT15, SW48, SW480, RKO, and LS1034) from which RNA was isolated by Trizol (Invitrogen, Carlsbad, Calif., USA). Ten primary colorectal carcinoma samples and ten normal colorectal samples from cancer patients were also included, from which RNA was isolated by the All prep DNA/RNA mini kit (Qiagen) and the Ribopure™ kit (Applied Biosystems/Ambion, Foster City, Calif., USA).

### Publicly Available Databases

Sequence information about genes and their different transcripts have been investigated using the Ensembl genome browser and all herein described sequences are in compliance with release 50, published July 2008. Sequence specificities, on the other hand, have been assessed by BLAST. These searches were carried out in the human genomic plus transcript database, by use of the nucleotide blast program, and the megablast algorithm.

### Exon Microarray Analysis

The GeneChip® Human Exon 1.0 ST Array (Affymetrix, Santa Clara, Calif., USA) provides genome-wide detection of RNA expression at both gene and exon levels. The microarray has approximately 5.4 million probes grouped into 1.4 million probesets examining more than a million known and predicted exons. The probes are distributed in the different exons along the entire transcript length, and for a gene with ten exons, there are roughly 40 probes matching its sequence. With probes in different exons along the transcript it is possible to monitor the level of expression for each exon compared with the others in the gene and thereby detect different transcript variants created after events such as alternative splicing and alternative promoter usage or poly-adenylation sites.

Ten normal colonic tissue samples, ten colorectal cancer tissue samples and six colorectal cancer cell lines (HT29, HCT15, SW48, SW480, RKO, and LS1034) were analysed. Raw data were imported into the XRAY software (version 2.81; Biotique Systems Inc., Reno, Nev., USA) where quantile normalisation and calculation of probeset expression values were performed and summarized. Only “core” probesets (RefSeq and full-length GenBank mRNAs) were analysed and the expression score for a probeset was defined to be the median of its probe expression scores. For each probeset the log2-ratio of expression level in test samples to that observed in control samples were calculated.

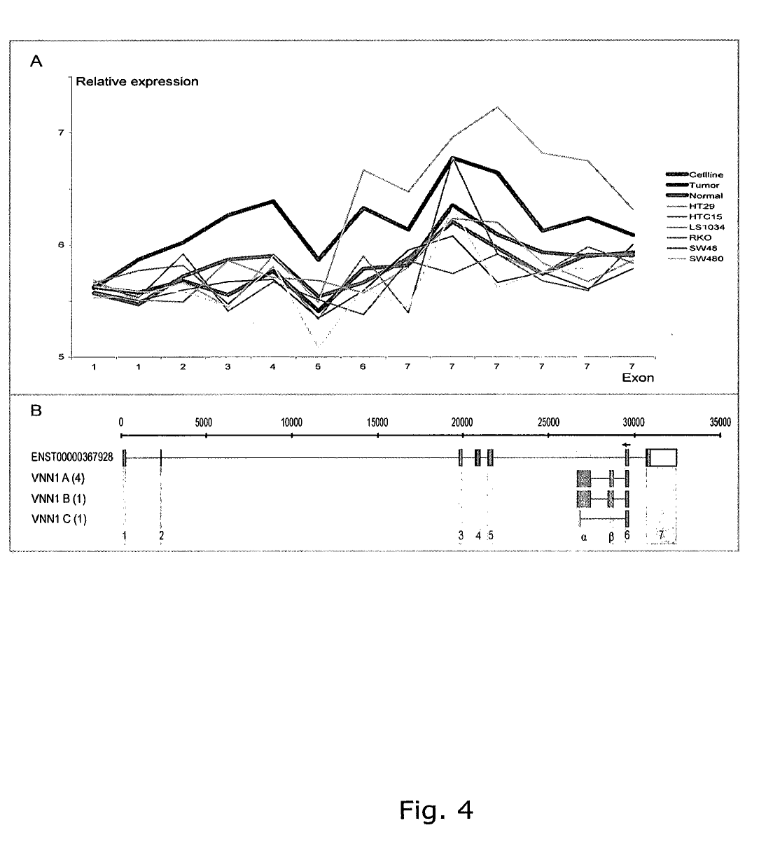

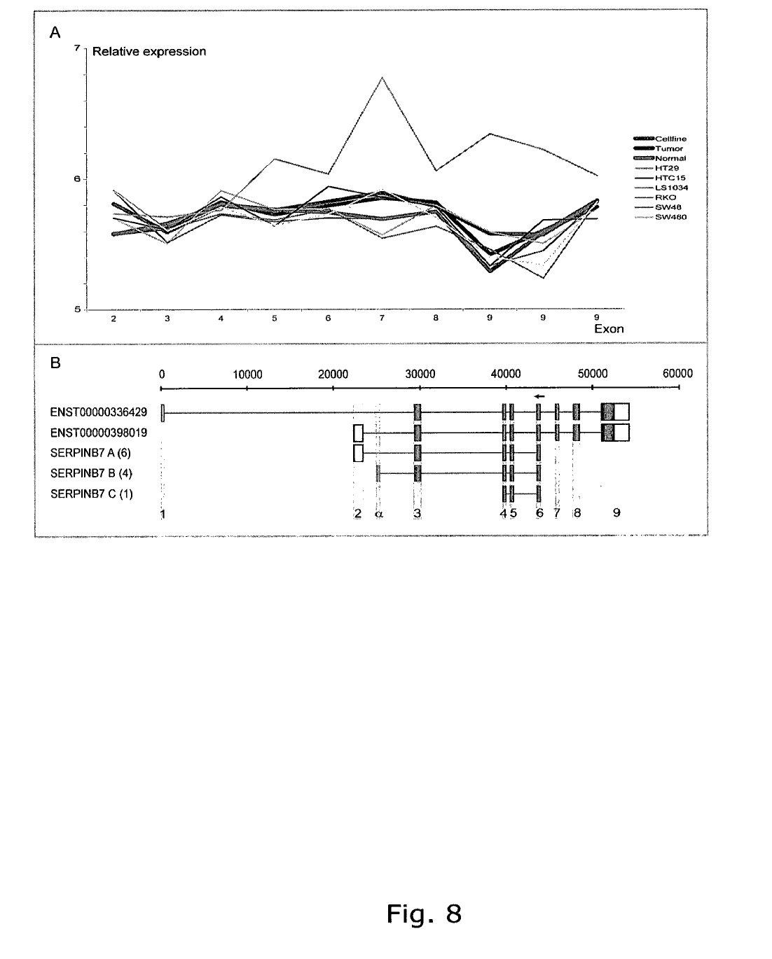

Exon microarray data were investigated from genes resulting from all the three different input strategies (outlier expression profiles, known and putative fusion genes, and ETS family members). The longitudinal exon expression profile along the entire transcript length of each gene was visualized by an in-house created visual basics script, and evaluated manually by looking for profiles where individual samples were overexpressed only in the 3′ part of the transcript compared to the rest of the samples (examples in FIG. 4 and FIG. 8). Genes with this type of profile were investigated further in the laboratory with 5′-RACE, cloning and sequencing.

Rapid Amplification of cDNA Ends

The complete 5′- and 3′-ends of cDNA can be amplified by PCR, using a technique variously called rapid amplification of cDNA ends (RACE), one-sided PCR and anchored PCR. The technique uses PCR to amplify partial cDNAs that represent the region between the 5′- or 3′-end and a single point in an mRNA transcript. The main requirement is that a short stretch of sequence in the mRNA of interest is known. A gene-specific primer (GSP), oriented in the direction of either the 5′- or 3′-end, is designed to anneal in the already known sequence. Extension of the cDNA from the end and back to the known region is achieved by using a primer annealing to the pre-existing poly(A) region (3′-RACE) or to an appended homopolymer tail or linker (5′-RACE).

### 5′-RACE

In this project 5′-RACE was performed using the SMART RACE cDNA Amplification kit (Clontech, Mountain View, Calif., USA). The first-strand synthesis is primed with an oligo-(dT) primer and performed by a Moloney murine leukemia virus reverse transcriptase (MMLV RT) which adds 3-5 residues (predominantly cytosines) upon reaching the 3′-end of the first-strand cDNA. A SMART II A oligo in the reaction mix contains a terminal stretch of G-residues which anneals to this cDNA tail. MMLV RT switches template from the mRNA to the SMART oligo and generates a complete cDNA copy of the mRNA with the additional SMART sequence at the end. MMLV RT's terminal transferase activity is most efficient when the enzyme has reached the end of the RNA-template and the SMART sequence is therefore typically added only to complete first-strand cDNAs.

The 5′-end of the cDNA can then be amplified using a universal primer (UP) which anneals in the SMART sequence and a primer specific for the gene of interest. The GSP must be between 23 and 25 nucleotides long, have a GC-content between 50 and 70 percent, and an annealing temperature above 70° C.

On occasion, a reverse transcription reaction can be non-specifically primed and result in a cDNA containing the SMART sequence at both ends. To reduce the likelihood of such aberrant products, a mixture of long and short UPs (with excess of the short UP) is used. The long UP contains inverted repeat elements. During PCR of a cDNA with SMART sequence in both ends, the long UP will anneal in both ends and the inverted repeats anneal to each other, making a panhandle-like structure. This blocks amplification of such aberrant products because the short UPs are unable to anneal.

Generation of 5′-RACE-ready cDNA was performed using the SMART RACE cDNA amplification kit (Clontech) and PrimeScript reverse transcriptase (Takara Bio Inc., Otsu, Shiga, Japan). One μg total RNA was combined with 2.4 μM oligo-(dT) primer, 2.4 μM SMART II A oligo, and sterile water to a total volume of 5 μl. The reaction mix was first incubated at 70° C. for 2 min to allow the primers to anneal and then on ice for two minutes before adding 1× first-strand buffer, 2 mM dithiothreitol (DTT), 1 mM dNTP, and 200 U PrimeScript reverse transcriptase to a total volume of 10 μl. Elongation of the cDNA at 42° C. for 90 min followed. The first-strand reaction was then diluted in 100 μl Tricine-EDTA buffer and the reaction was stopped by incubation at 72° C. for 7 min.

RACE reactions were performed using the SMART RACE cDNA amplification kit and the Advantage 2 PCR kit (Clontech). 1× Advantage 2 PCR buffer, 0.2 mM dNTP mix, 1× Advantage 2 PCR polymerase mix, 2.5 μl RACE-ready cDNA, 1× Universal primer mix (UPM), 0.2 μM GSP, and PCR-grade water was combined to a final volume of 50 μl. The cycling conditions were as described in Table 1.

Nested RACE was then performed by combining the same reagents as for RACE, but this time with 5 μl diluted RACE product as template and nested primers. The nested RACE was run by 25 cycles of 30 sec at 94° C., 30 sec at 68° C., and 3 min at 72° C.

### Cloning

Cloning and transformation was performed using the TOPO TA Cloning Kit (Invitrogen). This kit takes advantage of topoisomerase I and the fact that it can bind to DNA and cleave the phosphodiester backbone after 5′-CCCTT-3′. The energy from the broken bond is conserved by formation of a covalent bond between the cleaved strand and the topoisomerase I. Before cloning, the vector is cut into linear form, with single 3′ thymidine (T) overhangs. Taq polymerase has a non-template dependent terminal transferase activity, which adds a single deoxyadenosine (A) to the 3′-ends of PCR products. By reversing the cleavage reaction the PCR product with its A-overhang is readily incorporated into the T-overhang containing vector and the topoisomerase is released.

The vector contains the lethal ccdB gene fused to the LacZα gene. Ligation of the PCR product disrupts expression of the ccdB-LacZα gene and allows only positive recombinants to grow. A gene for ampicillin resistance in the vector ensures that only transformed bacteria will grow in the presence of this antibiotic compound.

Four μl PCR product eluted from an agarose gel was mixed with 1 μl salt solution and 1 μl TOPO vector before incubation at room temperature for 30 min. The cloning reaction was then transferred to ice. Two μl of the reaction was transferred to a vial of One Shot TOP10 E. coli and incubated on ice for 5-30 min. The cells were given a heat shock for 30 sec at 42° C. and immediately transferred back to ice. 250 μl of room temperature S.O.C. medium was added and the cells incubated horizontally at 37° C. and 200 rpm for 1 h. After the incubation 50 μl and 75 μl of the transformation mix was spread on pre-warmed selective LB plates containing 100 μg/ml ampicillin. The plates were incubated over night at 37° C.

Individual colonies were picked from selective plates and used to inoculate individual cultures consisting of 5 ml LB-medium and 10 μl ampicillin. The cultures were incubated at 37° C. and 250 rpm over night. Bacterial cells were then harvested by centrifugation and plasmid DNA was purified using the QIAprep Spin Miniprep kit (Qiagen).

### DNA Sequencing

The sequencing reaction was performed in a 96-well Optical Reaction Plate and consisted of purified template DNA (either PCR product eluted from agarose gel or plasmid DNA from Miniprep purification), primer (forward or reverse), BigDye Terminator v3.1 or v1.1 premix (Applied Biosystems), BigDye Sequencing buffer (Applied Biosystems) and Milli-Q water to a total volume of 10 μl. First, the reaction mixes were incubated at 96° C. for 2 min, followed by 25 thermal cycles of 15 sec at 96° C., 5 sec at 50° C., and 4 min at 60° C. The thermal cycling was performed on an MJ Research Cycler (BIO-RAD).

The BigDye Terminator v3.1 premix was used when the fragment to be sequenced were longer than 500 base pairs and the v1.1 for shorter fragments. The premix contains dNTPs and ddNTPs. The different ddNTPs are modified with fluorescent labels which emit light at specific wavelengths when exposed to a laser beam. This makes it possible to visualise the different bases.

### Product Purification

After the sequencing reaction unincorporated dye terminators, salts and other charged molecules must be removed. This was done by using the BigDye Xterminator Purification Kit (Applied Biosystems). Forty-five μl of SAM™ solution and 10 μl of Xterminator™ were added to the sequencing reaction after completion of thermal cycling. The reaction mixes were then vortexed for 30 min and briefly centrifuged in the end.

The SAM solution enhances the performance of the Xterminator solution and stabilises the post-purification reactions. The Xterminator, on the other hand, scavenges unincorporated dye terminators and free salts.

### Capillary Analysis

The 96-well Optical Reaction Plate was sealed with a 3100 Genetic Analyzer Plate Septa (Applied Biosystems), placed in a 96-well Plate Base, and inserted into a fully automated AB 3730 DNA analyser (Applied Biosystems). Inside the analyser the 48-capillary array is filled with POP7 polymer (Applied Biosystems). The samples are then loaded and separated according to size as they migrate through the polymer-filled capillaries. As the fluorescently labelled DNA fragments reach the detection window, a laser beam excites the dye molecules and causes them to fluoresce. The Data Collection software reads and interprets the fluorescence data before displaying them as an electropherogram. The samples were analysed using the software Sequencing Analysis 5.2 (Applied Biosystems), and all electropherograms were read both manually and automatically.

### Example 1

### Identification of Novel Transcripts

Three starting points were used for the candidate gene selection in the hunt for fusion genes; genes with outlier expression profiles, known and putative 3′ fusion gene partners and members of the ETS gene family.

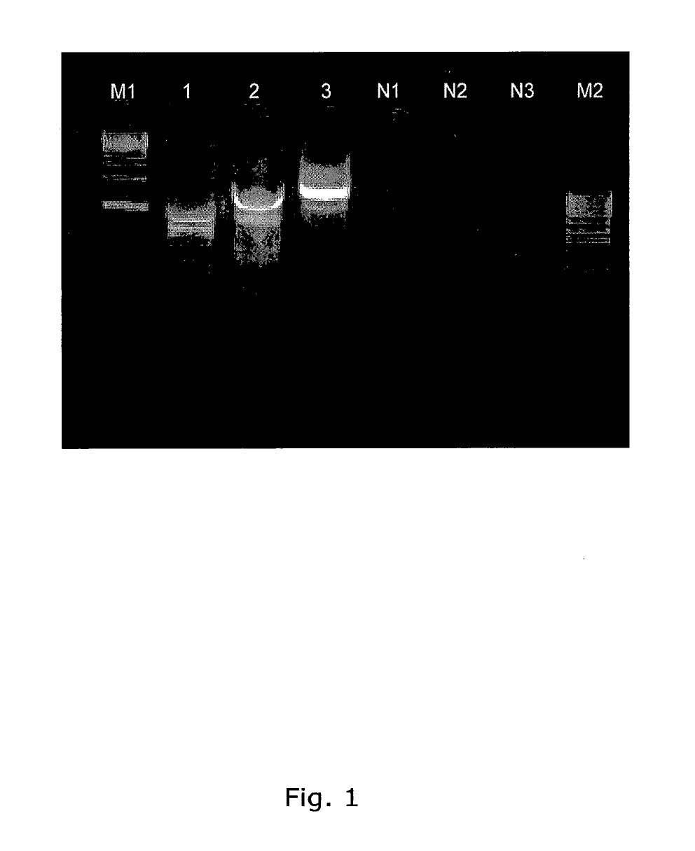

Here, 508 genes (131 outliers, 349 known and putative fusion genes and 28 ETS family members) were investigated with the exon microarray. Eleven genes (RAD51L1, NKAIN2, VNN1, C4BPB, HOXC11, TFR2, SERPINB7, TFPT, GJB6, PRRX1, and PRRX2) had a longitudinal profile along the exons where one or two of the cell lines deviated from the rest only in the 3′-end. Five of these genes (TFR2, SERPINB7, C4BPB, VNN1, and GJB6) had outlier expression profiles in colorectal tissues, and the other six genes (PRRX1, PRRX2, NKAIN2, HOXC11, TFPT, and RAD51L1) are known fusion gene partners. None of the ETS family members and none of the putative fusion genes exhibited the desirable profile. For each of the 11 genes 5′-RACE and nested RACE was performed (see FIG. 1 for representative results). Products were separated with gel electrophoresis, cut and eluted from the gel, cloned, and sequenced. No fusion genes were found from analysis of these genes, but novel transcript variants were found in all of the 11 genes.

The exon expression profile of RAD51L1 in the SW48 cell line deviated from the other cell lines by having higher expression from exon seven and throughout the gene (FIG. 2A). Five transcript variants with a total of 14 exons are known for RAD51L1, but sequencing of the 5′-RACE products from SW48 revealed six novel transcript variants which all included novel exons located inside intron number seven (FIG. 2B). The novel exons are spliced together in different ways to create the different transcripts. See Appendix II for details about each transcript and the different exons. The nucleotide sequences of the novel transcripts were evaluated by use of the Translate tool for translation of nucleotide sequences into protein sequences. This revealed that the transcripts B and F contain open reading frames (i.e., a start codon which is not followed by an immediate in-frame stop codon) of 66 amino acids, and these are thus potentially protein-coding.

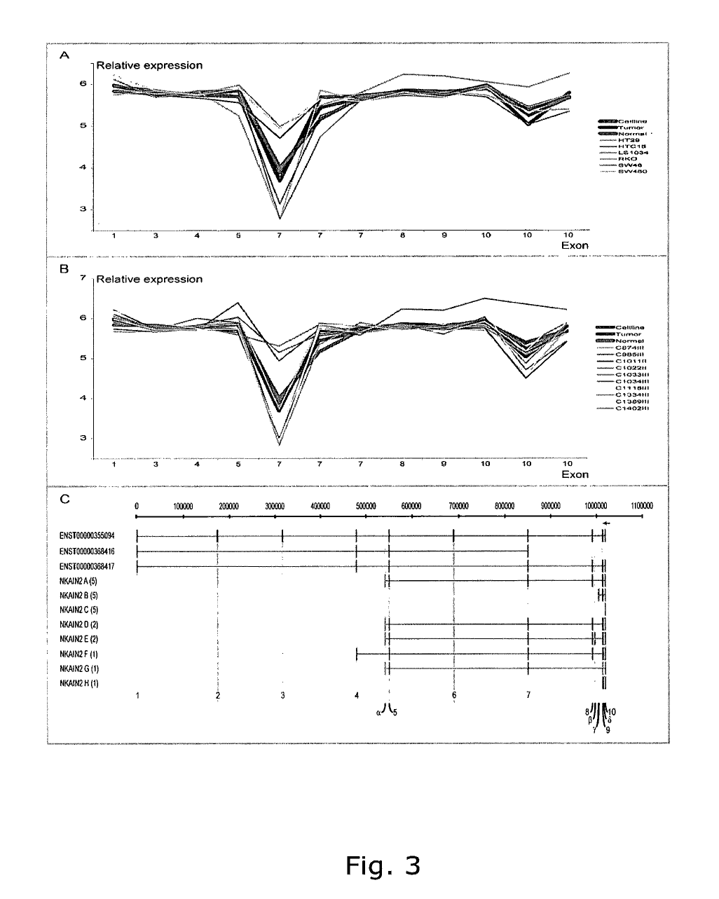

The same type of exon expression profile was found for NKAIN2 in both a cell line (LS1034) and a primary tumour (C1033III). These profiles show a higher expression of exons eight, nine, and ten in L51034 (FIG. 3A) and C1033III (FIG. 3B) compared to the other cell lines and primary tumours.

Three transcripts are known for NKAIN2, all of which are transcribed from the same promoter (FIG. 3C). Sequencing of the 5′-RACE products from both LS1034 and C1033III reveals the presence of eight novel transcripts including four novel exons, here denoted α, β, γ, and δ. Exon α is used as first exon in transcripts A, D, E, and G whereas exon γ is the first exon in transcript B. Exons β and δ, on the other hand, are located downstream of exon eight and nine, respectively. In the different transcripts, transcription is initiated at exon α, four, γ, nine, or ten. The Translate tool reveals transcripts A, G, D, F, and E as potentially protein-coding, with open reading frames of up to 173 amino acids, whereas transcripts C, B, and H probably are not.

The exon expression profile for VNN1 in the cell line HT29 deviated from that of the other cell lines by higher expression of exons six and seven (FIG. 4A). Sequencing of the 5′-RACE products from VNN1 revealed three transcript variants in HT29 (FIG. 4B). One transcript variant with seven exons is known for VNN1, but exons one to five in this transcript were never detected in HT29, instead two new exons, α and β, located inside intron number five are present. Transcript A consists of exon α followed by exon β and exon six. The Translate tool indicates that the transcript might encode a protein of 83 amino acids. Transcript B is quite similar to A, but with a 35 basepairs longer exon β. This results in frame shift from the subsequent exon of transcript A, introducing a stop codon, and B is therefore most likely non-coding. In transcript C a short exon α is directly followed by exon six. The Translate tool revealed no open reading frame from this sequence.

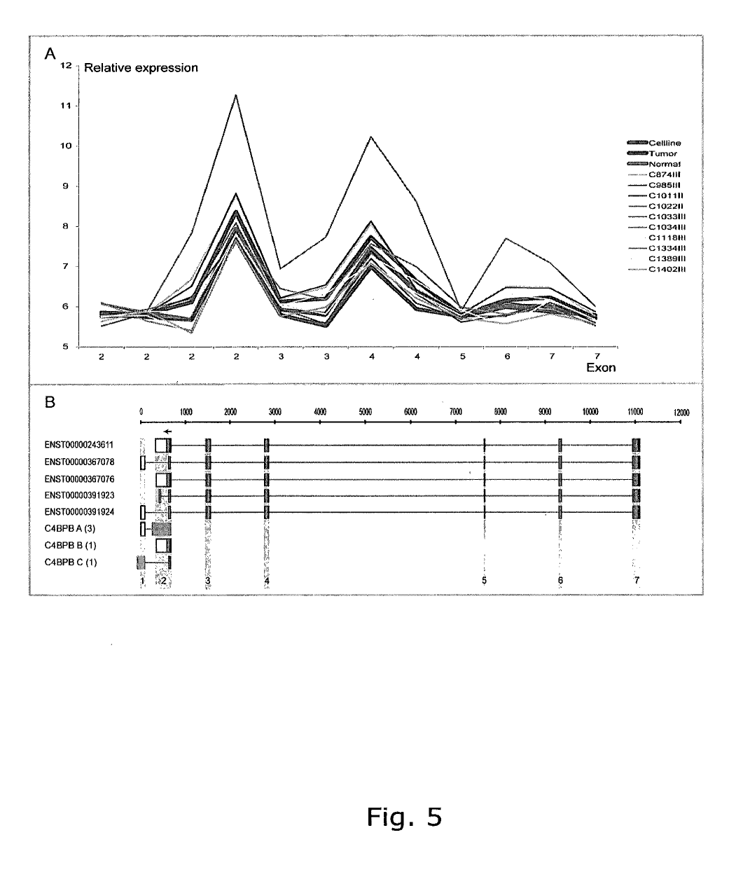

The exon expression profile for C4BPB in C1034III deviated from the other primary tumours by higher expression from the middle of the second exon and throughout the gene (FIG. 5A). Five different transcripts, transcribed from two different promoters, are known for C4BPB (FIG. 5B). Three different transcripts were found by sequencing of the 5′-RACE products from C1034III, all of which seem to be transcribed from the two known promoters (FIG. 5B). Transcript A consists of the reference exon one and an enlarged exon two with additional sequences 5′ to the reference exon. Transcript B starts in exon two, in accordance with both ENST00000243611 and ENST00000367076. Transcript C is similar to ENST00000367078, but with a larger first exon. Since the gene-specific primer is located relatively close to the 5′-end of the gene, we do not have enough information on whether the two new transcripts, A and C, are protein-coding.

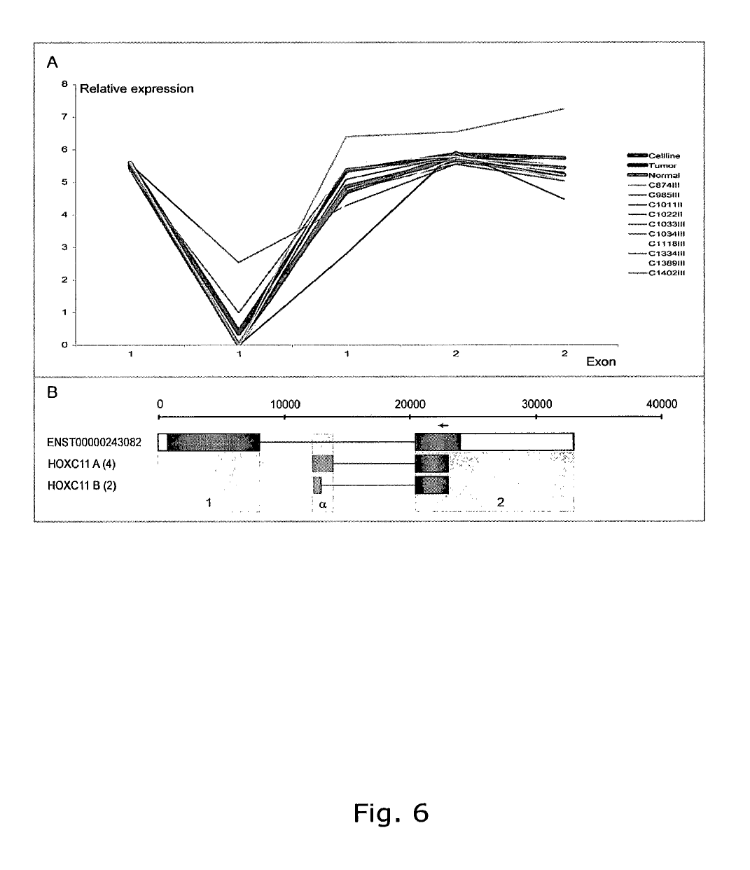

The exon expression profile for HOXC11 in the primary tumour C1402III deviates from the profile of the other tumours with higher expression from the end of exon one and throughout the gene (FIG. 6A). One transcript with two exons is known for HOXC11 (FIG. 6B). Sequencing of the 5′-RACE products revealed two novel transcripts in C1402III (FIG. 6B). These transcripts consist of a novel exon, here denoted α, of variable length, spliced to exon two in the known transcript. The Translate tool indicates that transcript A, with the large exon α, exhibits an open reading frame encoding up to 119 amino acids with multiple possible initiation codons. The C-terminal end of the putative peptide generated from transcript A is identical to the C-terminal end of the peptide generated from ENST00000243082. Transcript B has a short exon α and only a quite short open reading frame encoding 38 amino acids, identical to the last part of the open reading frame in transcript A.

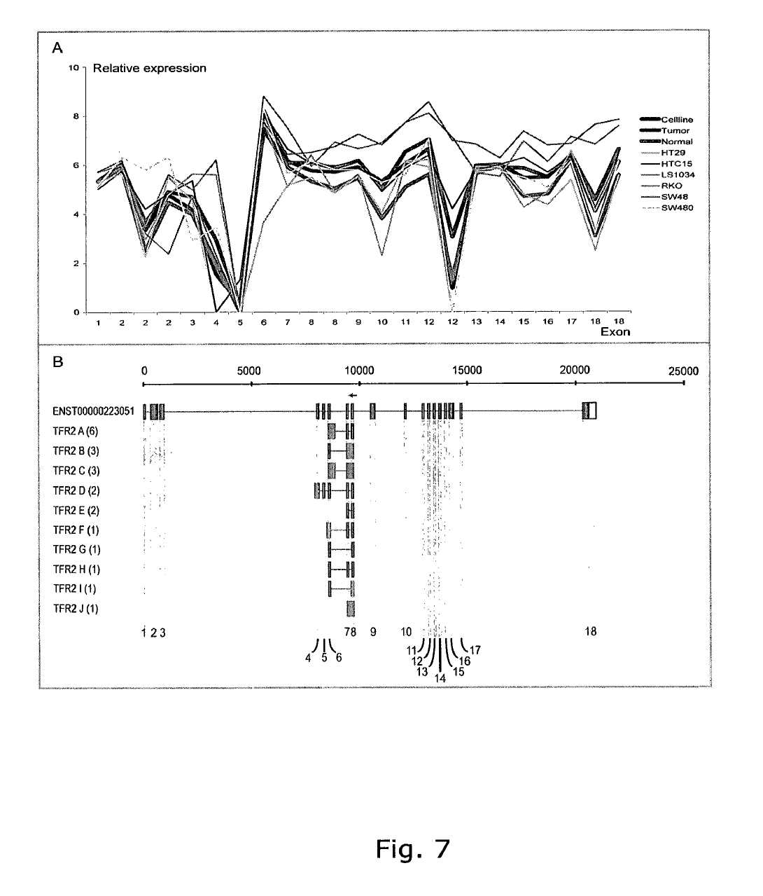

Two cell lines, RKO and SW48, had similar exon expression profiles for TFR2. These profiles deviated from those seen in the other cell lines by higher expression of exon eight and throughout the gene (FIG. 7A). One transcript with 18 exons is known for TFR2, and sequencing of the 5′-RACE products from RKO and SW48 revealed ten novel transcripts (FIG. 7B). Exons one, two, and three were never present in these transcripts, and instead, all transcripts were initiated from exons four, six, and seven. The transcripts differ with regard to the amount of intron sequence included around the known exons. The Translate tool indicates an open reading frame in transcripts A, E, F, and H encoding 46 amino acids and an open reading frame encoding 160 amino acids in transcript D. For all these five transcripts, no stop codon is encoded and the open reading frame continues into the exon(s) downstream of the primer location. No open reading frames were found for transcripts B, C, G, I, and J.

The exon expression profile of SERPINB7 in the LS1034 cell line deviated from the other cell lines in exons five to nine (FIG. 8A). Two transcript variants are known for SERPINB7 with a total of nine exons, where the first two are non-coding (FIG. 8B). Sequencing of the 5′-RACE products revealed three variants in LS1034. Transcript B exhibits a novel first exon located inside intron number two. The Translate tool indicates that the transcript variant encodes the same protein as the two known transcripts, but has a different 5′-UTR. Transcript A is identical to ENST00000398019. Transcript C only includes exons four to six and the Translate tool reveals that no open reading frame is encoded by the transcript.

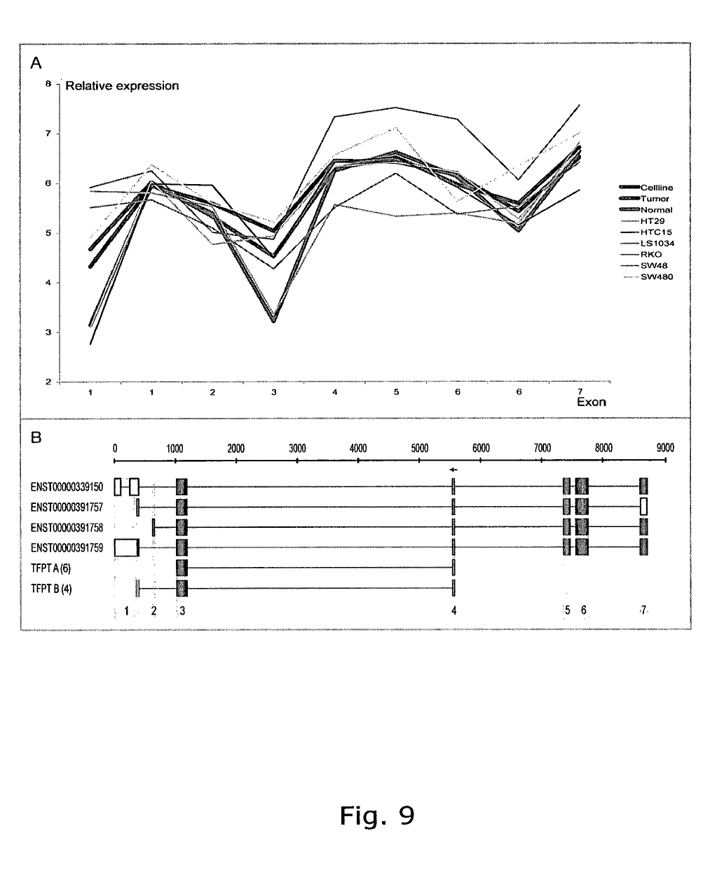

The exon expression profile for TFPT in SW48 shows higher expression in exons four, five, six, and seven compared to the other cell lines (FIG. 9A). Four transcripts, transcribed from three different promoters and with a total of seven exons, are known for TFPT (FIG. 9B). Sequencing of the 5′-RACE products revealed the presence of two transcripts in SW48 (FIG. 9B). Transcript A is transcribed from exon three and the Translate tool indicates that no open reading frame is encoded by the transcript. Transcript B, on the other hand, is similar to one of the known transcripts (ENST00000301757), but with a larger first exon.

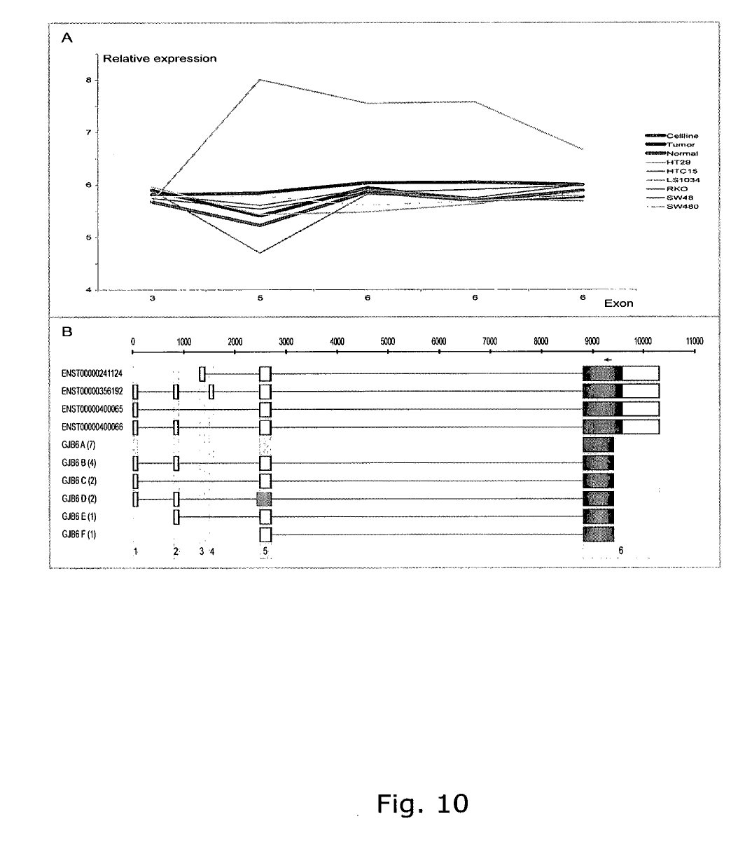

The exon expression profile for GJB6 in HT29 deviated from the other cell lines by having higher expression in exons five and six (FIG. 10A).

Four transcripts with a total of six exons are known for GJB6. Sequencing of the 5′-RACE products revealed the presence of six transcript variants in HT29 (FIG. 10B). Transcript A only includes the last exon, and do not encode an open reading frame. Transcripts B and C, are identical to two of the known protein-coding variants (ENST00000400066 and ENST00000400065, respectively). Transcript D presents the same exon composition as ENST00000400066 but the sequence of exon five is 21 basepairs longer on its 5′-end, which induces seven new amino acids upstream of the coding region. Transcript E and F are initiated in exons two and five, respectively, and the Translate tool indicates that they encode an intact protein, but have a different 5′-UTR.

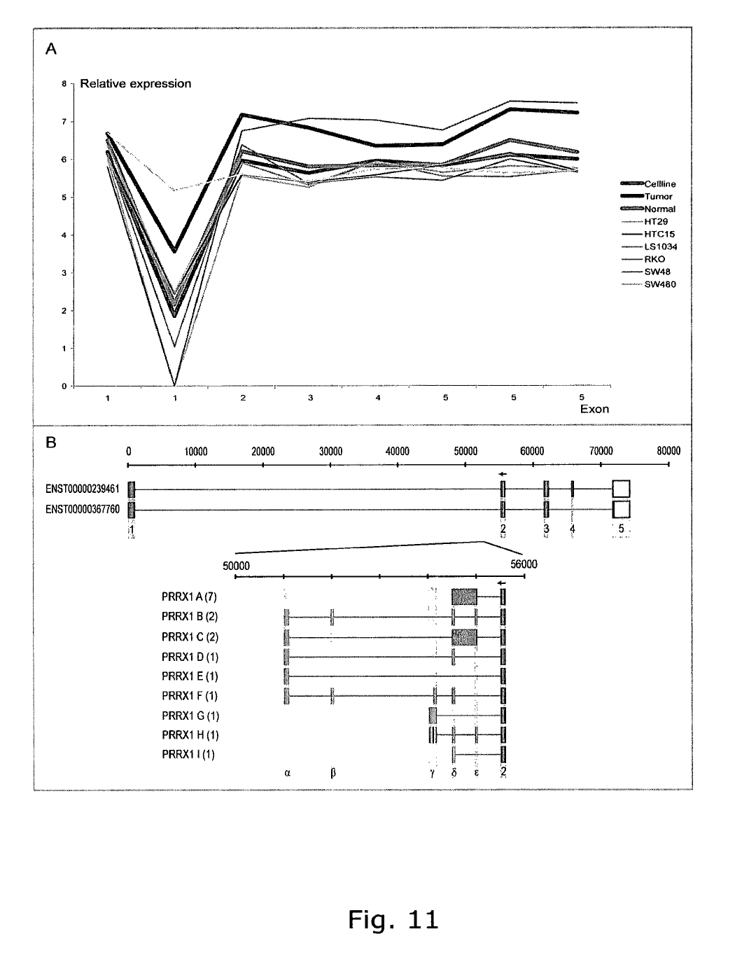

The exon expression profile for PRRX1 revealed higher expression of exons two to five in SW48 as compared to the other cell lines (FIG. 11A). Two transcripts with a total of five exons are known for PRRX1, and sequencing of the 5′-RACE products from SW48 revealed nine transcript variants with a total of five novel exons localised in the 3′-end of intron one (FIG. 11B). Exon one is not present in any of the transcripts, and instead, transcription is initiated at exons α, γ, and δ. The novel exons are spliced together in multiple ways to create the nine different transcript structures identified. The Translate tool indicates the presence of open reading frames in transcripts A and B which might encode up to 83 amino acids. No stop codons were found in these frames, indicating the presence of more coding exon(s) 3′ of the primer location. None of the other transcripts seem to contain open reading frames.

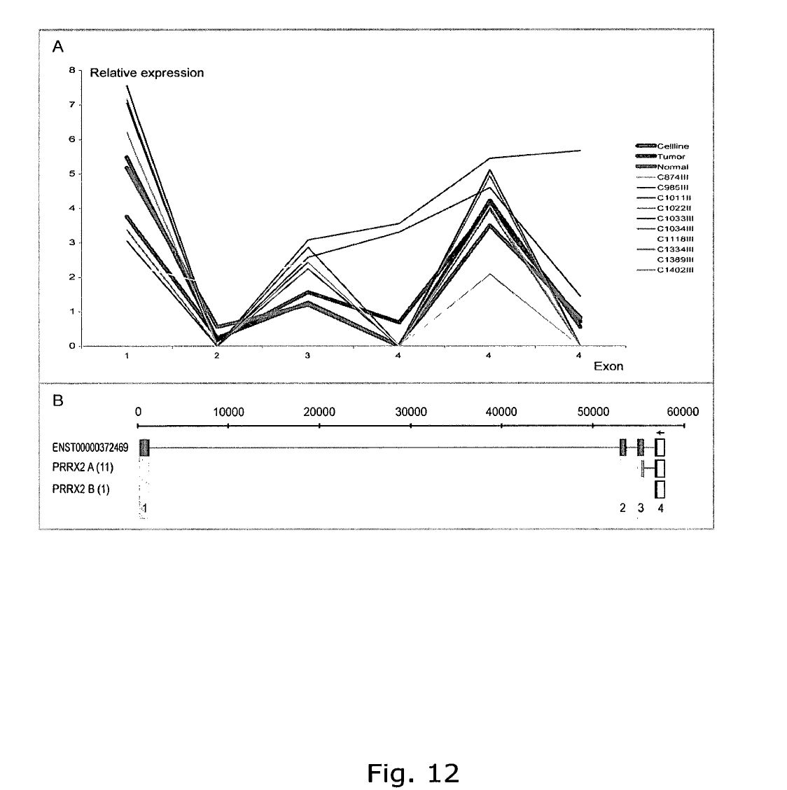

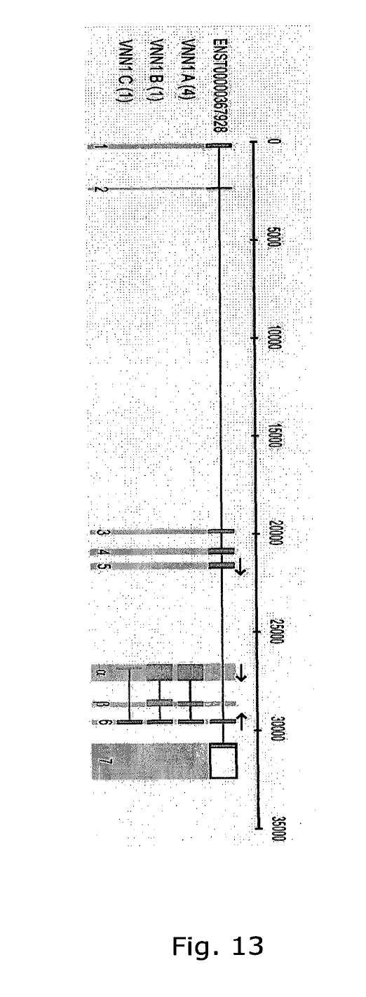

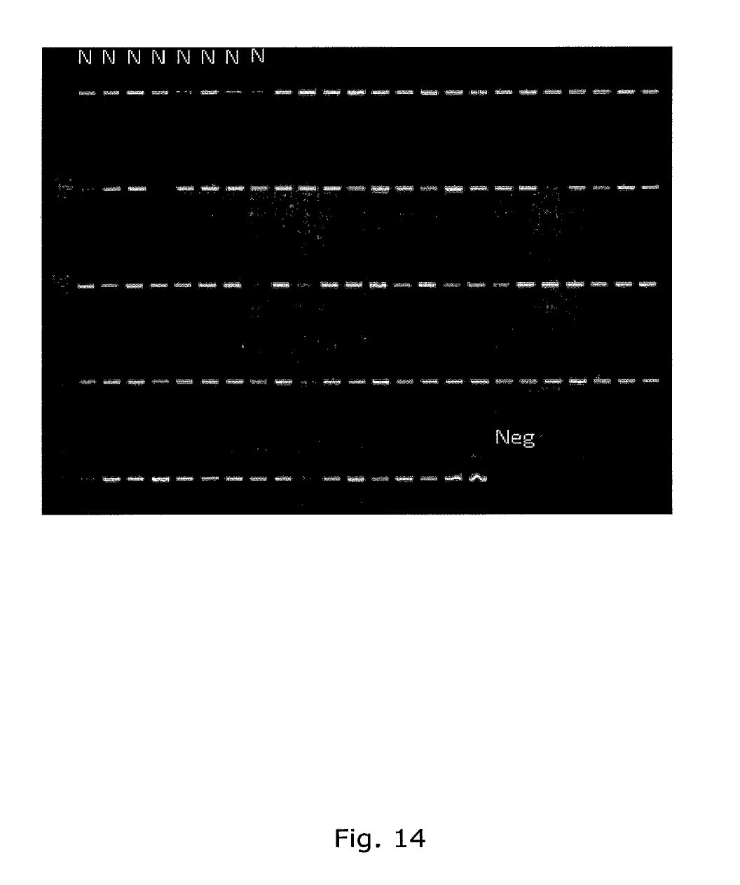

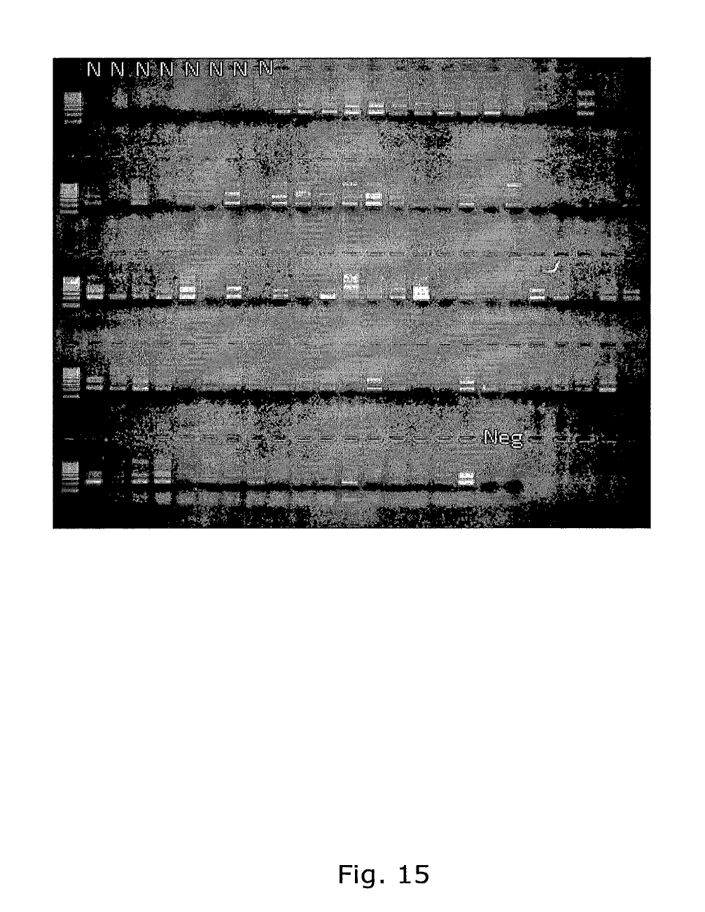

The exon expression profile for PRRX2 in the primary tumour sample C1033III deviates from the other samples by having higher expression in the last exon of the gene (FIG. 12A). One transcript, consisting of four exons, is known for PRRX2, and sequencing of the 5′-RACE products revealed two novel transcript variants, A and B (FIG. 12B). Transcript A includes parts of exon three spliced to exon four, whereas transcript B only consists of exon four. Eleven clones exhibited transcript A, and transcription was initiated at the exact same location for all clones (Appendix II). The Translate tool indicates that none of the transcripts are protein-coding.

### Methodological Considerations

Three starting points were used for the candidate gene selection in the search for novel transcript variants, including a search for fusion genes; genes with outlier expression profiles, known and putative 3′ fusion gene partners and members of the ETS gene family. A fusion gene usually leads to the overexpression of the downstream fusion partner and a fusion gene is usually only present in a subset of cancer samples. The formation of a fusion gene therefore leads to overexpression of the downstream partner gene in only some of the samples, giving rise to an outlier expression profile. Previously, cancer outlier profile analysis has been used to calculate outlier profiles in the search for novel fusion genes (Tomlins et al., Science 2005). Known and putative 3′ fusion gene partners and ETS gene family members were included because of their known susceptibility for undergoing rearrangements and because the same fusion genes (and in particular the same fusion gene partners) can be present in different cancer types.

Analysis of the longitudinal exon expression profile turned to be an important step in the process of enriching for genes with alterations in their transcript structures. If every exon in a gene is under the control of the same promoter, we would expect the exon expression levels to be similar throughout the gene. If, on the other hand, a gene has a second promoter or is the downstream partner in a fusion gene (and thus has downstream exons under the control of a new promoter), the exons downstream of the new promoter/breakpoint will be under the control of a different promoter than the upstream exons. The 5′-portion of the original gene is therefore regulated by one promoter and the 3′-portion by another, leading to different expression of the two parts. This may give rise to longitudinal exon expression profiles looking like the ones seen in FIG. 2A to FIG. 12A, where exons in the 3′-end of a gene have higher expression than the 5′-exons in certain samples as compared to others.

To investigate the transcript structure upstream of the altered exon expression, 5′-RACE was used. One debate concerning RACE methods is whether the entire beginning of the transcript is reached. For the SMART RACE kit used in the present project, it has been reported that 70-90% of the products correspond to the actual 5′-end of the mRNA. The majority of transcripts found in this project may therefore be considered to include the 5′-end of the mRNA. This is also supported by findings shown in Appendix II, where different clones for the same transcript start at the exact same base, indicating that this is the first base to be transcribed into mRNA. An example can be seen in Appendix II for PRRX2 transcript A, where all eleven clones started with the same nucleotide.

Multiple transcripts are found for the majority of genes. The gene-specific primers used in the RACE setup anneal to a particular exon. By use of the exon microarray expression profiles, gene-specific primers could be designed to anneal in exons indicated to be highly expressed, and therefore most likely also included in a potential novel transcript variant initiated from a novel and strong promoter.

Two steps in the process from mRNA to sequenced 5′-cDNA ends were essential for success: Firstly, since the RACE method only applies one gene-specific primer, it is necessary to perform nested RACE with a nested gene-specific primer to ensure gene-specific RACE products. Secondly, it is necessary to separate nested RACE products on an agarose gel, followed by elution of individual bands, prior to cloning. Abrogating this step will favour cloning of short products. Some of the adenosine overhangs produced by the PCR reaction, and necessary for cloning into the TOPO vectors, are lost during the gel elution step, thus making the cloning reaction less effective. Accordingly, the amount of transformation mix had to be increased to ensure sufficient growth of transformed bacteria.

### Novel Exons and Transcripts

Among the 11 genes investigated in the laboratory because of exon expression profiles deviating in the 3′-end of the transcripts, five were initially included as candidate genes due to outlier expression profiles in tissues from colorectal cancer (TFR2, SERPINB7, C4BPB, VNN1, GJB6) and six due to their known participation as fusion gene partners in other cancer types (RAD51L1, NKAIN2, HOXC11, TFPT, PRRX1, and PRRX2). In total, laboratory investigations of the 11 genes lead to the discovery of 57 novel transcript variants, including 22 novel exons and 34 putative novel promoters in colorectal cancer. In the following each gene and its transcript variants will be discussed in more detail.

Large discrepancies are seen in different human genome databases with regards to, for instance, what is considered a transcript variant and the nomenclature of exons and transcripts. Therefore, throughout the project one genomic database, Ensembl, have been used to asses the different transcripts and exons known for a given gene. Ensembl, which is curated by the European Bioinformatics Institute, is considered a comprehensive, well-annotated and stable database, where annotated genes and transcripts are based on mRNA and protein sequences deposited into public databases from the scientific community.

For RAD51L1, the transcription start sites of the herein identified novel transcript variants indicate the presence of three novel promoters, at exons denoted α, β, and γ. The exon expression profile for RAD51L1 (FIG. 2) shows higher expression of the last exons in the investigated cell line as compared to the others and therefore indicate that one or both of the alternative promoters are more activated than the reference promoters. The investigated cell line, SW48, also has higher expression of exon two compared to the other cell lines. This can not be explained by the transcripts described in this project because exons one to seven are not present in any of them. The high expression in exon number two might be explained by transcripts which do not contain exon eight, and therefore are not detected with the RACE primed for this exon.

For NKAIN2, the novel exon α is used as first exon in four of the sequenced transcripts and indicate the presence of a novel promoter. Promoters might also be present at exons four, γ, nine and ten, as these are the first exons in the other four transcripts. The exon expression profiles of the cell line and tumour sample investigated deviate most strikingly from the other cell lines and tumour samples in exon eight, nine, and ten. In addition, they both also have the highest expression in exon five, as compared to samples of the same kind, which is in line with the presence of this exon in five transcripts.

The exon expression profile for VNN1 in HT29 was quite striking (FIG. 4) with the higher expression of exons six and seven as compared to the average expression of the ten tumour samples, which are somewhat upregulated compared to normal samples and cell lines. Three transcript variants with two novel exons were found. For all novel transcript variants of VNN1 expression starts in the novel exon α, indicating the presence of a novel promoter. To account for the high expression of exons six and seven, the promoter used to generate these transcripts must be more active than the normal promoter in VNN1.

The enlarged exon two seen in transcript A of C4BPB might constitute a longer 5′-UTR and thereby affect its stability and/or regulation of translation. Transcript C might be the same as ENST00000367078. The first exon is bigger in transcript C, but this might be due to use of different TSSs and thus, the promoter is not necessarily a novel one.

Both of the novel transcripts seen for HOXC11 consist of a version of exon α, spliced to exon two in the reference transcript. This indicates the presence of a novel promoter at exon α. The possible protein encoded by transcript A, might be a truncated version of the known protein product of ENST00000243082 or a novel protein with identical C-terminal end.

The novel transcript D seen in TFR2 consists of exons four to eight and was only found in the RKO cell line. The exon expression profiles for the two investigated cell lines deviate most from the other cell lines in exons eight to ten, but the presence of exon four in transcript D is in concordance with the peak seen at this position in the exon expression profile for RKO. The drop in expression seen for exon five for all cell lines might be due to a non-functioning probeset. All transcripts are initiated from either exon four, six, or seven, indicating the presence of novel promoters in these regions.

Two novel and one known transcripts were found for SERPINB7 (FIG. 8). For SERPINB7, the first exon seen in transcript B is likely non-coding and can give the potentially encoded protein a different 5′-UTR than the known isoforms of the gene. This might affect the stability and regulation of the encoded protein.

The exon expression profile for TFPT in SW48 shows high expression of exon one, but lower expression of exons two and three. Exon two is not present in the two transcripts seen in SW48 and might therefore explain the drop in the expression profile. Exon three, on the other hand, is present in both transcripts. This drop in expression is seen, in various degrees, in this location for all the cell lines and may be due to a probeset not working properly. The enlarged first exon in transcript B might be due to alternative TSS use as compared to the known transcript, and not indicate the presence of a novel promoter.

The entire coding region of GJB6 is located in exon 6. The enlarged fifth exon seen in transcript D alters the 5′-UTR and might therefore affect the stability and/or regulation of translation. Transcripts E and F differ from the reference transcripts and indicate the presence of new promoters in front of exons two and five, respectively. The potential proteins encoded by these transcripts are identical, but the transcripts exhibit different 5′-UTR as compared to the known proteins and might therefore be regulated differently. None of the transcripts sequenced from the HT29 cell line includes exon 3, thus explaining the drop seen at this position in the exon expression profile.

In the novel transcript variants seen for PRRX1, transcription is initiated at exons α, γ, and δ indicating the presence of three novel promoters. The exon expression profile for the investigated cell line shows continuous high expression of PRRX1 in exons three, four, and five. This indicates the presence of all these exons in the full-length transcripts and is in concordance with the lack of stop codons upstream of the primer location in transcripts A and B. To account for the elevated expression of exons two to five, one or more of the novel promoters found in the investigated cell line must be more active than the normal promoter for PRRX1.

Eleven clones containing transcript A of PRRX2 were sequenced, all of which were of the exact same length because transcription was initiated at the exact same nucleotide. This indicates that the far 5′-end of the transcripts were reached using 5′-RACE and therefore also supports the findings of a wider repertoire of promoters for the other genes investigated in this project.

The Translate tool used to translate nucleotide sequences to peptide sequences of potential proteins has been used to evaluate whether or not different transcripts have the possibility to be protein-coding. The transcripts referred to as non-coding have been of two types; either with many stop codons dispersed throughout the nucleotide sequence, in all three reading frames, or a transcript sequence with no start codon. The latter type was found in transcripts from TFR2, SERPINB7, TFPT, and GJB6. The nucleotide sequences from these transcripts were typically containing an open reading frame, but did not include start codon for this frame. Nevertheless, these transcripts may as well represent sequences where the 5′-end of the cDNA has not been reached.

True non-coding transcripts may as well be functionally relevant to the cells. Over the past few years, several long non-coding RNAs have been discovered. Many of these RNAs control the activity of protein-coding genes and do so in a variety of ways without necessarily being dependent on the exact sequence of the RNA. For example, as seen from the DHFR gene, a non-coding RNA generated from one promoter in a gene can regulate the transcription of protein-coding transcripts generated from another promoter within the same gene.

Nonsense-mediated mRNA decay represents a posttranscriptional process which selectively recognises and degrades mRNAs with truncated open reading frames.

The novel transcripts detected in this project are clearly not degraded, as their corresponding genes were included in the study based on high mRNA levels. This is yet another indication that they may have functional implications to the cells.

The transcripts described in this example display 34 potentially novel promoters. This includes both transcripts potentially encoding the reference proteins but containing different 5′-UTR (as seen for GJB6, transcripts E and F) and transcripts potentially encoding novel proteins (as seen for RAD51L1, transcripts B and F). Heterogeneous 5′-UTRs can affect the stability and translation efficiency of the mRNAs and thereby affect the amount of protein present in a cell, whereas isoforms of the same gene may have different functions. The potential proteins encoded by transcripts identified in this project may therefore introduce effects to a cancer cell which are different to those of the proteins encoded by the reference transcripts.

As seen from Appendix II, the exact TSSs for the same type of transcripts within different clones differ by some nucleotides. This is in accordance with the findings that most human promoters lack one distinct TSS, but instead consist of a series of closely located TSSs spread over around 50 to 100 basepairs. For some transcripts, the TSSs seen in Appendix II are separated by more than 100 basepairs, and may therefore indicate the presence of more than one core promoter.

Summarised, the exon expression levels for 508 genes were investigated. Eleven of the genes had deviating exon expression profiles indicating qualitative changes in the transcript structure and were therefore investigated in the laboratory. No new fusion gene was found, but 57 novel transcript variants including 22 novel exons and 34 putative promoters were identified from colorectal cancer cell lines and tissue samples. Thus, in conclusion, we consider our novel strategy for identification of novel transcript variants in colorectal cancer as successful. The novel transcripts will be further investigated in our laboratory to elucidate their prevalence and clinical relevance in colorectal cancer, as well as their cancer-specificity.

### Example 2

According to the latest version of Ensembl (release 56, September 2009), there is only one transcript annotated for VNN1. This variant, ENST00000367928, has seven exons. Three new transcript variants were found by sequencing of the 5′-RACE products from HT29 and include two new exons inside intron number five of ENST00000367928. To distinguish transcripts including the novel exons from the ENST00000367928 transcripts, an RT-PCR assay was developed with two specific forward primers and a common reverse primer. The forward primer targeting ENST00000367928 is specific to the annotated exon 5 and the forward primer targeting the novel transcripts target the common region in exon {alpha}.

RT-PCR of VNN1 with primers specifically binding to the ENST00000367928 exon 5 to 6 from 8 normal colon mucosa (marked “N”), 105 colorectal cancers, 2 negative controls (marked “Neg”). PCR-product at the expected length was detected for all samples.

RT-PCR of novel exons within VNN1 with one of the primers specifically binding to the novel exon {alpha} and the exon 6 in ENST00000367928 from 8 normal colon mucosa (marked “N”), 105 colorectal cancers, 2 negative controls (marked “Neg”). PCR-products at the expected lengths according to the 5′RACE experiments, and one additional band, were detected for 87% of the colorectal cancers, but not for any of the normal colon mucosa or the negative controls.

All three transcripts (VNN1 A, B and C) originate partly from within the genomic portion annotated as intron 5, between exons 5 (ENSE00000764053) and 6 (ENSE00000764052), of the VNN1 gene (ENSG00000112299; ENST00000367928). VNN1-intron 5 is located 133,005,645 to 133,013,361 basepairs from the p-telomere of chromosome 6 (Ensembl release 56). The VNN1 gene is transcribed from the minus-strand; hence, the sequence starts further away from the p-telomere than it ends. The start and end positions of the transcripts can be found in Table-A-II-3.

### Tables

TABLE 1

| Cycling conditions for 5′-RACE. | |
|---|---|
| Temperature | Time |

| | | | |
|---|---|---|---|
| 94° C. | 30 sec | | |
| | | {close oversize brace} | 5 cycles |
| 72° C. | 3 min | | |
| 94° C. | 30 sec | | |
| 70° C. | 30 sec | {close oversize brace} | 5 cycles |
| 72° C. | 3 min | | |
| 94° C. | 30 sec | | |
| 68° C. | 30 sec | {close oversize brace} | 25 cycles |
| 72° C. | 3 min | | |

| | |
|---|---|
| 72° C. | 7 min |

TABLE APPENDIX I
Primers

| Gene | Name | Type | Length | Sequence | Tm (° C.) | GC (%) |
|---|---|---|---|---|---|---|
| ABL1 | ABL1_ex2_rev | Reverse | 20 | ACCCTGAGGCTCAAAGTCAG | 59.5 | 55 |
| ABL1 | ABL1_ex3_rev | Reverse | 23 | TTCCCCATTGTGATTATAGCCTA | 64.0 | 39 |
| BCR | BCR_ex1_forw | Forward | 20 | CAACAGTCCTTCGACAGCAG | 59.6 | 55 |
| BCR | BCR_ex13_forw | Forward | 21 | CAGATGCTGACCAACTCGTGT | 64.0 | 52 |
| BIRC5 | BIRC5-6′FAM-R | Reverse | 20 | TCTCCGCAGTTTCCTCAAAT | 59.8 | 45 |
| BIRC5 | BIRC5-EX2-L-F | Forward | 19 | GAGGCTGGCTTCATCCACT | 60.4 | 58 |
| BIRC5 | BIRC5-EX1- F | Forward | 20 | AGAACTGGCCCTTCTTGGAG | 60.8 | 55 |
| BIRC5 | BIRC5-EX2-K-F | Forward | 20 | GCCCAGTGTTTCTTCTGCTT | 59.5 | 50 |
| BIRC5 | BIRC5_ex4_Rev | Reverse | 20 | TCTCCGCAGTTTCCTCAAAT | 59.8 | 45 |
| C4BPB | C4BPB_ex1_F | Forward | 26 | CCTTGCTGGGAAGCCCTAACTCTGGA | 71.7 | 58 |
| C4BPB | C4BPB_ex2_R | Reverse | 25 | ACGCAACCATAAGACAGCACGCACA | 70.6 | 52 |
| C4BPB | C4BPB_ex2_nest_R | Reverse | 25 | GGCTGGAATTCACCCAGCTCAGACA | 70.5 | 56 |
| CST1 | CST1_ex1_F | Forward | 25 | TGCGGGTACTAAGAGCCAGGCAACA | 70.9 | 56 |
| CST1 | CST1_ex3_R | Reverse | 24 | CGAATGGCCTGGCACAGATCCCTA | 71.0 | 58 |
| CST1 | CST1_ex3_nest_R | Reverse | 27 | TGACACCTGGATTTCACCAGGGACCTT | 71.7 | 52 |
| ETV6 | ETV6_ex5_forw | Forward | 20 | CACTCCGTGGATTTCAAACA | 59.5 | 45 |
| FZD10 | FZD10_ex1_F | Forward | 25 | TTTATGCTGCTGGTGGTGGGGATCA | 71.3 | 52 |
| FZD10 | FZD10_ex1_R | Reverse | 25 | CCGTGGTGAGTTTTCTGGGGATGCT | 71.3 | 56 |
| FZD10 | FZD10_ex1_nest_R | Reverse | 25 | GCCGCCAGGATCTTCCAGTAATCCA | 71.3 | 56 |
| GJB6 | GJB6_ex3_F | Forward | 25 | TTCGGATAGAGGGGTCGCTGTGGTG | 72.1 | 60 |
| GJB6 | GJB6_ex3_R | Reverse | 25 | GCAGCATGCAAATCACAGACGCAGA | 71.2 | 52 |
| GJB6 | GJB6_ex3_nest_R | Reverse | 25 | AACAAGGTTGGGGCAGGGGTCAATC | 72.0 | 56 |
| GPR177 | GPR177_ex2_R | Reverse | 20 | GGAGGGGAATGTGAACAGAA | 57.0 | 50 |
| GPR177 | GPR177_ex1_F | Forward | 20 | TCTGCTCGTGTTCCAAATCA | 57.1 | 45 |
| HOXB13 | HOXB13_ex1_F | Forward | 25 | CAGCCAGATGTGTTGCCAGGGAGAA | 71.2 | 56 |
| HOXB13 | HOXB13_ex2_R | Reverse | 25 | CTTGCGCCTCTTGTCCTTGGTGATG | 70.9 | 56 |
| HOXB13 | HOXB13_ex2_R_alt2 | Reverse | 28 | TAAGGGGTAGCGCTGTTCTTCACCTTGG | 72.5 | 54 |
| HOXC11 | HOXC11_ex1_F | Forward | 25 | ACAAATCCCAGCTCGTCCGGTTCAG | 71.4 | 56 |
| HOXC11 | HOXC11_ex2_R | Reverse | 25 | CCCTGGCCACAGTCCAGTTTTCCAC | 71.6 | 60 |
| HOXC11 | HOXC11_ex2_nest_R | Reverse | 25 | CCGGTCTGCAGGTTACAGCAGAGGA | 70.6 | 60 |
| Hs.446400 | Hs.446400_F | Forward | 20 | CAGAGCTGCATCCTTATGGT | 55.1 | 50 |
| Hs.446400 | Hs.446400_R | Reverse | 20 | AGCTGCAAGTTGTTGTTCCA | 56.5 | 45 |
| MIER1 | MIER1_ex9_F | Forward | 22 | CCATCAGAAGACTGGAAAAAGG | 58.3 | 45 |
| MIER1 | MIER1_ex10_R | Reverse | 22 | TGCTTCTACACCCTTCTCATCA | 57.5 | 45 |
| MTHFD2L | MTHFD2L_ex5_F | Forward | 20 | GACCCAAGAGTCAGCGGTAT | 56.5 | 55 |
| MTHFD2L | MTHFD2L_ex7_R | Reverse | 20 | GATCTTCCAGCCACAACCAC | 57.4 | 55 |
| NKAIN2 | NKAIN2_ex8_F | Forward | 27 | TGGCTATCAAGGGCCTCAGAAGACATC | 70.0 | 52 |
| NKAIN2 | NKAIN2_ex10_R | Reverse | 25 | CAGGAAATCCAAGATGGGCGTGTCC | 71.5 | 56 |
| NKAIN2 | NKAIN2_ex10_nest_R | Reverse | 25 | CAAGTGGAATTGGTGTGTGCGTGCT | 70.0 | 52 |
| PBX1 | PBX1_ex3_rev | Reverse | 21 | TGCTCCACTGAGTTGTCTGAA | 59.1 | 48 |
| PBX1 | PBX1_ex5_rev | Reverse | 20 | GGGTTGCTGAGATGGGAATA | 59.9 | 50 |
| PRRX1 | PRRX1_ex_F | Forward | 25 | TAGACCTGGAGGAAGCCGGGGACAT | 71.9 | 60 |
| PRRX1 | PRRX1_ex_R | Reverse | 25 | TAATCGGTGGGTCTCGGAGCAGGAC | 71.3 | 60 |
| PRRX1 | PRRX1_ex_nest_R | Reverse | 25 | GTGTCCGCTCAAAGACACGCTCCAA | 71.4 | 56 |
| PRRX1 | PRRX1_int1_ex3_R | Reverse | 25 | CCCAGCTTTGGTGGCACTTCTGTGA | 71.3 | 56 |
| PRRX1 | PRRX1_int1_ex4_R | Reverse | 28 | TCAGGGAAAACGTGAAACTCCTCTTGTC | 69.2 | 46 |
| PRRX2 | PRRX2_ex3_F | Forward | 25 | GCCCACCGCCCTGAGTCCAGATTAT | 72.2 | 60 |
| PRRX2 | PRRX2_ex4_R | Reverse | 25 | AGGTCCTTGGCAGGCTCTTCCACCT | 71.4 | 60 |
| PRRX2 | PRRX2_ex4_nest_R | Reverse | 25 | CAAGGGTTGTGGGCTGCAGTCTCTG | 71.0 | 60 |
| RAD51L1 | RAD51L1_ex5_F | Forward | 27 | CCCACCAACATGGGAGGATTAGAAGGA | 71.2 | 52 |
| RAD51L1 | RAD51L1_ex8_R | Reverse | 25 | AGCTGGAGACACCAGGTCTGCCTGA | 70.3 | 60 |
| RAD51L1 | RAD51L1_ex8_nest_R | Reverse | 25 | CTGAGAAGCCAGGGCTCCACTCAGA | 70.0 | 60 |
| RUNX1 | RUNX1_ex2_rev | Reverse | 20 | CGTGGACGTCTCTAGAAGGA | 58.0 | 55 |
| SERPINB7 | SERPINB7_ex3_F | Forward | 25 | TTGGGCGCTCAAGATGACTCCCTCT | 71.2 | 56 |
| SERPINB7 | SERPINB7_ex5_R | Reverse | 25 | GTCAACTCGCTCCACTTTGGCATCG | 70.9 | 56 |
| SERPINB7 | SERPINB7_ex6_R | Reverse | 26 | GAAGGCTGATTGCCACTTGCCTTTGA | 71.5 | 50 |
| TCF3 | TCF3_ex15_forw | Forward | 19 | CACCCTCCCTGACCTGTCT | 60.0 | 63 |
| TCF3 | TCF3_ex17_forw | Forward | 20 | GTGACATCAACGAGGCCTTT | 60.1 | 50 |
| TFPT | TFPT_ex3_F | Forward | 25 | CACATCCTGGAGAGCGAGCTGGAGA | 70.8 | 60 |
| TFPT | TFPT_ex4_R | Reverse | 25 | TCCTGCTGCAGCCTCCGAGTTATCC | 71.7 | 60 |
| TFPT | TFPT_ex4_nest_R | Reverse | 24 | CCTGTTCAGGACCCGCTCGTTCAC | 70.8 | 63 |
| TFR2 | TFR2_ex7_F | Forward | 25 | TCAGGACTTCGGGGCTCAAGGAGTG | 71.6 | 60 |
| TFR2 | TFR2_ex8_R | Reverse | 25 | GCTGGGAAGGCCTGATGATGCAACT | 71.5 | 56 |
| TFR2 | TFR2_ex8_nest_R | Reverse | 25 | TGTAGGGGTCTCCAGTTCCCAGGTG | 69.4 | 60 |
| Universal | NUP | Forward | 23 | AAGCAGTGGTATCAACGCAGAGT | 60.8 | 48 |
| Universal | UPM-Long | Forward | 45 | CTAATACGACTCACTATAGGGCAAGCAGT | 80.4 | 47 |
| | | | | GGTATCAACGCAGAGT | | |
| Universal | UPM-Short | Forward | 22 | CTAATACGACTCACTATAGGGC | 51.3 | 45 |
| USP11 | USP11_eks6_R | Reverse | 18 | GCCTGGCTGACCCTTGAA | 58.8 | 61 |
| USP11 | USP11_ex5_F | Forward | 18 | GAGCGGTTTCTGGTGGAG | 55.7 | 61 |
| VNN1 | VNN1_ex5_F | Forward | 25 | TGCACACTGTGGAAGGGCGCTATTA | 69.7 | 52 |
| VNN1 | VNN1_ex7_R | Reverse | 27 | GGCTTCAGACTAAACAAGCGTCCGTCA | 70.8 | 52 |
| VNN1 | VNN1_ex6_nest_R | Reverse | 25 | CTGGGTTCCGAAAGTGCCACTGAGG | 71.8 | 60 |
| WIF1 | WIF1_ex9_F | Forward | 26 | GAACCTGCCATGAACCCAACAAATGC | 71.4 | 50 |
| WIF1 | WIF1_ex10_R | Reverse | 25 | GCCGCTCCTCGGCCTTTTTAAGTGA | 72.5 | 56 |
| WIF1 | WIF1_ex9_nest_R | Reverse | 25 | ATGGCAGGTTCCATGTGCACCACAG | 71.7 | 56 |
| ZDHHC20 | ZDHHC20_ex1_F | Forward | 18 | CTGGAGCGTCCGAGTCAC | 56.3 | 67 |
| ZDHHC20 | ZDHHC20_ex2_R | Reverse | 22 | CAACGGTCTTTCCATTTTCTTC | 58.5 | 41 |

### Tables-Appendix-II

### Abbreviations:

T=Primary tumour sample
C=Cell line
N=Number of times sequenced

TABLE A-II-1
Exon positions from RAD51L1.

| | | | Exon 1 | | Exon 2 | | Exon 3 | | Exon 4 | |
|---|---|---|---|---|---|---|---|---|---|---|
| Sequence identifier | N | C | Start | End | Start | End | Start | End | Start | End |
| ENST00000342389 | | | 1 | 62 | 3,751 | 3,836 | 5,673 | 5,786 | 15,289 | 15,405 |
| ENST00000344360 | | | | | 3,753 | 3,836 | 5,673 | 5,786 | 15,289 | 15,405 |
| ENST00000390683 | | | | | 3,753 | 3,836 | 5,673 | 5,786 | 15,289 | 15,405 |
| ENST00000402498 | | | 1 | 62 | 3,751 | 3,836 | 5,673 | 5,786 | 15,289 | 15,405 |
| ENST00000403044 | | | 1 | 62 | 3,751 | 3,836 | 5,673 | 5,786 | 15,289 | 15,405 |
| RAD51L1 A | 2 | SW48 | | | | | | | | |
| RAD51L1 B | 1 | SW48 | | | | | | | | |
| RAD51L1 C | 1 | SW48 | | | | | | | | |
| RAD51L1 D | 1 | SW48 | | | | | | | | |
| RAD51L1 E | 1 | SW48 | | | | | | | | |
| RAD51L1 F | 1 | SW48 | | | | | | | | |
| | | | Exon 5 | | Exon 6 | | Exon 7 | | Exon α | |
| Sequence identifier | N | C | Start | End | Start | End | Start | End | Start | End |
| ENST00000342389 | | | 45,212 | 45,348 | 66,078 | 66,197 | 67,230 | 67,413 | | |
| ENST00000344360 | | | 45,212 | 45,348 | 66,078 | 66,197 | 67,230 | 67,413 | | |
| ENST00000390683 | | | 45,212 | 45,348 | 66,078 | 66,197 | 67,230 | 67,413 | | |
| ENST00000402498 | | | 45,212 | 45,348 | 66,078 | 66,197 | 67,230 | 67,413 | | |
| ENST00000403044 | | | 45,212 | 45,348 | 66,078 | 66,197 | 67,230 | 67,413 | | |
| RAD51L1 A | 2 | SW48 | | | | | | | | |
| RAD51L1 B | 1 | SW48 | | | | | | | | |
| RAD51L1 C | 1 | SW48 | | | | | | | 170,719 | 170,815 |
| RAD51L1 D | 1 | SW48 | | | | | | | 170,771 | 170,815 |
| RAD51L1 E | 1 | SW48 | | | | | | | 170,771 | 170,815 |
| RAD51L1 F | 1 | SW48 | | | | | | | | |
| | | | Exon β | | Exon γ | | Exon δ | | Exon ε | |
| Sequence identifier | N | C | Start | End | Start | End | Start | End | Start | End |
| ENST00000342389 | | | | | | | | | | |
| ENST00000344360 | | | | | | | | | | |
| ENST00000390683 | | | | | | | | | | |
| ENST00000402498 | | | | | | | | | | |
| ENST00000403044 | | | | | | | | | | |
| RAD51L1 A | 2 | SW48 | 180,425 | 180,522 | | | | | | |
| RAD51L1 B | 1 | SW48 | 180,466 | 180,522 | | | | | 296,028 | 296,321 |
| RAD51L1 C | 1 | SW48 | | | | | | | | |
| RAD51L1 D | 1 | SW48 | | | | | | | | |
| RAD51L1 E | 1 | SW48 | | | | | | | 296,028 | 296,321 |
| RAD51L1 F | 1 | SW48 | | | 190,417 | 190,440 | 269,418 | 269,498 | 296,028 | 296,321 |

| | | |
|---|---|---|
| Exon ζ | Exon η | Exon 8 |

| Sequence identifier | N | C | Start | End | Start | End | Start | End | Start positions |
|---|---|---|---|---|---|---|---|---|---|
| ENST00000342389 | | | | | | | 472,093 | 472,189 | |
| ENST00000344360 | | | | | | | 472,093 | 472,189 | |
| ENST00000390683 | | | | | | | 472,093 | 472,189 | |
| ENST00000402498 | | | | | | | 472,093 | 472,189 | |
| ENST00000403044 | | | | | | | 472,093 | 472,189 | |
| RAD51L1 A | 2 | SW48 | | | | | 472,093 | 472,146 | 180,425, 180,459 |
| RAD51L1 B | 1 | SW48 | | | | | 472,093 | 472,146 | 180,466, 180,473 |
| RAD51L1 C | 1 | SW48 | 328,645 | 328,748 | 353,296 | 353,411 | 472,093 | 472,146 | 170,719 |
| RAD51L1 D | 1 | SW48 | | | | | 472,093 | 472,146 | 170,771 |
| RAD51L1 E | 1 | SW48 | | | | | 472,093 | 472,146 | 170,771 |
| RAD51L1 F | 1 | SW48 | | | | | 472,093 | 472,146 | 190,417 |

Start/end sequence positions are indicated relative to start of exon 1 (ENSG00000182185; transcribed from plus strand; start position: 67,356,262 bp from chromosome 14 p-telomere; Ensembl release 50).

TABLE A-II-2
Exon positions from NKAIN2.

| | | | Exon 1 | | Exon 2 | | Exon 3 | |
|---|---|---|---|---|---|---|---|---|
| Sequence identifier | N | C/T | Start | End | Start | End | Start | End |
| ENST00000355094 | | | 4 | 114 | 167,076 | 167,103 | 317,766 | 317,801 |
| ENST00000368416 | | | 1 | 114 | | | | |
| ENST00000368417 | | | 1 | 114 | | | | |
| NKAIN2 A | 5 | C1033III | | | | | | |
| | | and | | | | | | |
| | | LS1034 | | | | | | |
| NKAIN2 B | 5 | LS1034 | | | | | | |
| NKAIN2 C | 5 | C1033III | | | | | | |
| NKAIN2 D | 2 | C1033III | | | | | | |
| | | and | | | | | | |
| | | LS1034 | | | | | | |
| NKAIN2 E | 2 | LS1034 | | | | | | |
| NKAIN2 F | 1 | C1033 | | | | | | |
| NKAIN2 G | 1 | C1033III | | | | | | |
| NKAIN2 H | 1 | LS1034 | | | | | | |
| | | | Exon 4 | | Exon α | | Exon 5 | |
| Sequence identifier | N | C/T | Start | End | Start | End | Start | End |
| ENST00000355094 | | | 478,866 | 479,003 | | | 551,128 | 551,208 |
| ENST00000368416 | | | 478,866 | 479,003 | | | 551,128 | 551,208 |
| ENST00000368417 | | | 478,866 | 479,003 | | | 551,128 | 551,208 |
| NKAIN2 A | 5 | C1033III | | | 544,294 | 544,565 | 551,128 | 551,208 |
| | | and | | | | | | |
| | | LS1034 | | | | | | |
| NKAIN2 B | 5 | LS1034 | | | | | | |
| NKAIN2 C | 5 | C1033III | | | | | | |
| NKAIN2 D | 2 | C1033III | | | 544,294 | 544,565 | 551,128 | 551,208 |
| | | and | | | | | | |
| | | LS1034 | | | | | | |
| NKAIN2 E | 2 | LS1034 | | | 544,294 | 544,565 | 551,128 | 551,208 |
| NKAIN2 F | 1 | C1033 | 478,866 | 479,003 | | | 551,128 | 551,208 |
| NKAIN2 G | 1 | C1033III | | | 544,294 | 544,565 | 551,128 | 551,208 |
| NKAIN2 H | 1 | LS1034 | | | | | | |
| | | | Exon 6 | | Exon 7 | | Exon 8 | |
| Sequence identifier | N | C/T | Start | End | Start | End | Start | End |
| ENST00000355094 | | | 686,247 | 686,253 | 854,053 | 854,247 | 987,200 | 987,260 |
| ENST00000368416 | | | | | 854,047 | 854,819 | | |
| ENST00000368417 | | | | | 854,047 | 854,247 | 987,200 | 987,260 |
| NKAIN2 A | 5 | C1033III | | | 854,047 | 854,247 | 987,200 | 987,260 |
| | | and | | | | | | |
| | | LS1034 | | | | | | |
| NKAIN2 B | 5 | LS1034 | | | | | | |
| NKAIN2 C | 5 | C1033III | | | | | | |
| NKAIN2 D | 2 | C1033III | | | 854,047 | 854,247 | 987,200 | 987,260 |
| | | and | | | | | | |
| | | LS1034 | | | | | | |
| NKAIN2 E | 2 | LS1034 | | | 854,047 | 854,247 | 987,200 | 987,260 |
| NKAIN2 F | 1 | C1033 | | | 854,047 | 854,247 | 987,200 | 987,260 |
| NKAIN2 G | 1 | C1033III | | | 854,047 | 854,247 | | |
| NKAIN2 H | 1 | LS1034 | | | | | | |
| | | | Exon β | | Exon γ | | Exon 9 | |
| Sequence identifier | N | C/T | Start | End | Start | End | Start | End |
| ENST00000355094 | | | | | | | 1,014,248 | 1,014,329 |
| ENST00000368416 | | | | | | | | |
| ENST00000368417 | | | | | | | 1,014,248 | 1,014,329 |
| NKAIN2 A | 5 | C1033III | | | | | 1,014,248 | 1,014,329 |
| | | and | | | | | | |
| | | LS1034 | | | | | | |
| NKAIN2 B | 5 | LS1034 | | | 1,010,112 | 1,010,192 | 1,014,248 | 1,014,329 |
| NKAIN2 C | 5 | C1033III | | | | | | |
| NKAIN2 D | 2 | C1033III | | | | | 1,014,248 | 1,014,329 |
| | | and | | | | | | |
| | | LS1034 | | | | | | |
| NKAIN2 E | 2 | LS1034 | 990,705 | 990,747 | | | 1,014,248 | 1,014,329 |
| NKAIN2 F | 1 | C1033 | | | | | 1,014,248 | 1,014,329 |
| NKAIN2 G | 1 | C1033III | | | | | 1,014,291 | 1,014,329 |
| NKAIN2 H | 1 | LS1034 | | | | | 1,014,248 | 1,014,329 |

| | | | Exon δ | | Exon 10 | | |
|---|---|---|---|---|---|---|---|
| Sequence identifier | N | C/T | Start | End | Start | End | Start position |
| ENST00000355094 | | | | | 1,019,081 | 1,021,477 | |
| ENST00000368416 | | | | | | | |
| ENST00000368417 | | | | | 1,019,081 | 1,021,518 | |
| NKAIN2 A | 5 | C1033III | | | 1,019,081 | 1,019,288 | 544,470, 544,470, |
| | | and | | | | | 544,406, 544,470, |
| | | LS1034 | | | | | 544,294 |
| NKAIN2 B | 5 | LS1034 | | | 1,019,081 | 1,019,288 | 1,010,112 |
| | | | | | | | for all five |
| | | | | | | | clones |
| | | | | | | | 1,019,091, |
| | | | | | | | 1,019,226, |
| | | | | | | | 1,019,226, |
| | | | | | | | 1,019,226, |
| NKAIN2 C | 5 | C1033III | | | 1,019,081 | 1,019,288 | 1,019,081 |
| NKAIN2 D | 2 | LS1034 | 1,014,947 | 1015035 | 1,019,081 | 1,019,288 | 544,470, 544,455 |
| NKAIN2 E | 2 | LS1034 | 1,014,947 | 1015035 | 1,019,081 | 1,019,288 | clones |
| NKAIN2 F | 1 | C1033 | 1,014,947 | 1015035 | 1,019,081 | 1,019,288 | 478,941 |
| NKAIN2 G | 1 | C1033III | | | 1,019,081 | 1,019,288 | 544,470 |
| NKAIN2 H | 1 | LS1034 | 1,014,947 | 1015035 | 1,019,081 | 1,019,288 | 1,014,248 |

Start/end sequence positions are indicated relative to start of exon 1 (ENSG00000188580; transcribed from plus strand; start position: 124,166,985 bp from chromosome 6 p-telomere; Ensembl release 50).

TABLE A-II-3
Exon positions from VNN1.

| | | | Exon 1 | | Exon 2 | | Exon 3 | | Exon 4 | |
|---|---|---|---|---|---|---|---|---|---|---|
| Sequence identifier | N | C | Start | End | Start | End | Start | End | Start | End |
| ENST00000367928 | | | 1 | 224 | 2,211 | 2,342 | 19,868 | 20,061 | 20,735 | 21,027 |
| VNN1 A | 4 | HT29 | | | | | | | | |
| VNN1 B | 1 | HT29 | | | | | | | | |
| VNN1 C | 1 | HT29 | | | | | | | | |

| | | | Exon 5 | | Exon α | | Exon β | | Exon 6 | | |
|---|---|---|---|---|---|---|---|---|---|---|---|
| Sequence identifier | N | C | Start | End | Start | End | Start | End | Start | End | Start positions |
| ENST00000367928 | | | 21,466 | 21,828 | | | | | 29,545 | 29,716 | |
| VNN1 A | 4 | HT29 | | | 26,645 | 27,450 | 28,645 | 28,796 | 29,545 | 29,659 | 26,645, 26,670, |
| | | | | | | | | | | | 26,662, 26,676 |
| VNN1 B | 1 | HT29 | | | 26,645 | 27,450 | 28,610 | 28,796 | 29,545 | 29,659 | 26,675 |
| VNN1 C | 1 | HT29 | | | 26,680 | 26,788 | | | 29,545 | 29,659 | 26,680 |

Start/end sequence positions are indicated relative to start of exon 1 (ENSG00000112299; transcribed from minus strand; start position: 133,076,881 bp from chromosome 6 p-telomere; Ensembl release 50).

TABLE A-II-4
Exon positions from C4BPB.

| | | | Exon 1 | | Exon 2 | | |
|---|---|---|---|---|---|---|---|
| Sequence identifier | N | T | Start | End | Start | End | Start positions |
| ENST00000243611 | | | | | 372 | 723 | |
| ENST00000367076 | | | | | 372 | 723 | |
| ENST00000367078 | | | 1 | 80 | 615 | 723 | |
| ENST00000391923 | | | | | 416* | *723 | |
| ENST00000391924 | | | 1 | 80 | 615 | 723 | |
| C4BPB A | 3 | C1034III | 1 | 80 | 232 | 641 | 1, −13, −11 |
| C4BPB B | 1 | C1034III | | | 372 | 641 | 372 |
| C4BPB C | 1 | C1034III | −53 | 80 | 615 | 641 | −53 |

Start/end sequence positions are indicated relative to start of exon 1 (ENSG00000123843; transcribed from plus strand; start position: 205,328,835 bp from chromosome 1 p-telomere; Ensembl release 50).
*ENST00000391923 lacks base pairs 496-614

TABLE A-II-5
Exon positions from HOXC11.

| | | | Exon 1 | | Exon α | | Exon 2 | | |
|---|---|---|---|---|---|---|---|---|---|
| Sequence identifier | N | T | Start | End | Start | End | Start | End | Start positions |
| ENST00000243082 | | | 1 | 798 | | | 2,055 | 3,292 | |
| HOXC11 A | 4 | C1402III | | | 1,244 | 1,398 | 2,055 | 2,300 | 1,254, 1,281, 1,244, 1,244 |
| HOXC11 B | 2 | C1402III | | | 1,254 | 1,300 | 2,055 | 2,300 | 1,254, 1,254 |

Start/end sequence positions are indicated relative to start of exon 1 (ENSG00000123388; transcribed from plus strand; start position: 52,653,177 bp from chromosome 12 p-telomere; Ensembl release 50).

TABLE A-II-6
Exon positions from TFR2.

| | | | Exon 1 | | Exon 2 | | Exon 3 | | Exon 4 | |
|---|---|---|---|---|---|---|---|---|---|---|
| Sequence identifier | N | C | Start | End | Start | End | Start | End | Start | End |
| ENST00000223051 | | | 1 | 74 | 322 | 575 | 678 | 865 | 7,995 | 8,135 |
| TFR2 A | 6 | SW48 and | | | | | | | | |
| | | RKO | | | | | | | | |
| TFR2 B | 3 | SW48 | | | | | | | | |
| | | SW48 and | | | | | | | | |
| TFR2 C | 3 | RKO | | | | | | | | |
| TFR2 D | 2 | RKO | | | | | | | 7,938 | 8,135 |
| TFR2 E | 2 | RKO | | | | | | | | |
| TFR2 F | 1 | RKO | | | | | | | | |
| TFR2 G | 1 | SW48 | | | | | | | | |
| TFR2 H | 1 | RKO | | | | | | | | |
| TFR2 I | 1 | RKO | | | | | | | | |
| TFR2 J | 1 | SW48 | | | | | | | | |

| | | | Exon 5 | | Exon 6 | | Exon 7 | | Exon 8 | | |
|---|---|---|---|---|---|---|---|---|---|---|---|
| Sequence identifier | N | C | Start | End | Start | End | Start | End | Start | End | Start positions |
| ENST00000223051 | | | 8,211 | 8,322 | 8,428 | 8,550 | 9,353 | 9,469 | 9,606 | 9,745 | |
| TFR2 A | 6 | SW48 and | | | 8,428 | 8,772 | 9,353 | 9,469 | 9,606 | 9,633 | 8,541, 8,541, 8,549, |
| | | RKO | | | | | | | | | 8,542, 8,536, 8,546 |
| TFR2 B | 3 | SW48 | | | 8,428 | 8,550 | 9,353 | 9,605 | 9,606 | 9,633 | 8,498, 8,517, 8,517 |
| TFR2 C | 3 | SW48 and | | | 8428* | *8772 | 9353** | **9605 | 9,606 | 9,633 | 8,526, 8,549, 8,546 |
| | | RKO | | | | | | | | | |
| TFR2 D | 2 | RKO | 8,211 | 8,322 | 8,428 | 8,550 | 9,353 | 9,469 | 9,606 | 9,633 | 7,938, 7,938 |
| TFR2 E | 2 | RKO | | | | | 9,353 | 9,469 | 9,606 | 9,633 | 9,360, 9,395 |
| TFR2 F | 1 | RKO | | | 8,404 | 8,550 | 9,353 | 9,469 | 9,606 | 9,633 | 8,404 |
| TFR2 G | 1 | SW48 | | | 8,428 | 8,550 | | | 9,606 | 9,633 | 8,502 |
| TFR2 H | 1 | RKO | | | 8,428 | 8,550 | 9,353 | 9,469 | 9,606 | 9,633 | 8,502 |
| TFR2 I | 1 | RKO | | | 8,428 | 8,550 | 9,571 | 9,605 | 9,606 | 9,633 | 8,486 |
| TFR2 J | 1 | SW48 | | | | | 9,353 | 9,605 | 9,606 | 9,633 | 9,395 |

Start/end sequence positions are indicated relative to start of exon 1 (ENSG00000106327; transcribed from minus strand; start position: 100,077,109 bp from chromosome 7 p-telomere; Ensembl release 50).
*One clone lacks base pairs 8551-8714
**One clone lacks base pairs 9470-9570

TABLE A-II-7
Exon positions from SERPINB7.

| | | | Exon 1 | | Exon 2 | | Exon α | | Exon 3 | |
|---|---|---|---|---|---|---|---|---|---|---|
| Sequence identifier | N | C | Start | End | Start | End | Start | End | Start | End |
| ENST00000336429 | | | 1 | 78 | | | | | 29,313 | 29,498 |
| ENST00000398019 | | | | | 22,336 | 22,674 | | | 29,313 | 29,498 |
| SERPINB7 A | 6 | LS1034 | | | 22,336 | 22,674 | | | 29,313 | 29,498 |
| SERPINB7 B | 4 | LS1034 | | | | | 24,736 | 24,783 | 29,313 | 29,498 |
| SERPINB7 C | 1 | LS1034 | | | | | | | | |

| | | | Exon 4 | | Exon 5 | | Exon 6 | | |
|---|---|---|---|---|---|---|---|---|---|
| Sequence identifier | N | C | Start | End | Start | End | Start | End | Start positions |
| ENST00000336429 | | | 39,351 | 39,401 | 40,119 | 40,238 | 43,224 | 43,341 | |
| ENST00000398019 | | | 39,351 | 39,401 | 40,119 | 40,238 | 43,224 | 43,341 | |
| SERPINB7 A | 6 | LS1034 | 39,351 | 39,401 | 40,119 | 40,238 | 43,224 | 43,277 | 22,382, 22,336, |
| | | | | | | | | | 22,339, 22,339, |
| | | | | | | | | | 22,388, 22,495 |
| SERPINB7 B | 4 | LS1034 | 39,351 | 39,401 | 40,119 | 40,238 | 43,224 | 43,277 | 24,739, 24,739, |
| | | | | | | | | | 24,736, 24,739 |
| SERPINB7 C | 1 | LS1034 | 39,351 | 39,401 | 40,119 | 40,238 | 43,224 | 43,277 | 39,395 |

Start/end sequence positions are indicated relative to start of exon 1 (ENSG00000166396; transcribed from plus strand; start position: 59,571,257 bp from chromosome 18 p-telomere; Ensembl release 50).

TABLE A-II-8
Exon positions from TFPT.

| | | | Exon 1 | | Exon 2 | | Exon 3 | | Exon 4 | | |
|---|---|---|---|---|---|---|---|---|---|---|---|
| Sequence identifier | N | C | Start | End | Start | End | Start | End | Start | End | Start positions |
| ENST00000339150 | | | 19* | *429 | | | 976 | 1,234 | 5,552 | 5,622 | |
| ENST00000391757 | | | 388 | 429 | | | 976 | 1,234 | 5,552 | 5,622 | |
| ENST00000391758 | | | | | 602 | 636 | 976 | 1,234 | 5,552 | 5,622 | |
| ENST00000391759 | | | 1 | 429 | | | 976 | 1,234 | 5,552 | 5,622 | |
| TFPT A | 6 | SW48 | | | | | 976 | 1,234 | 5,552 | 5,575 | 1,117, 1,121, 1,118, |
| | | | | | | | | | | | 1,117, 1,114, 1,114 |
| TFPT B | 4 | SW48 | 331 | 429 | | | 976 | 1,234 | 5,552 | 5,575 | 331, 331, 331, 355 |

Start/end sequence positions are indicated relative to start of exon 1 (ENSG00000105619; transcribed from minus strand; start position: 59,310,867 bp from chromosome 19 p-telomere; Ensembl release 50).
*ENST00000339150 lacks base pairs 163-268

TABLE A-II-9
Exon positions from GJB6.

| | | | Exon 1 | | Exon 2 | | Exon 3 | |
|---|---|---|---|---|---|---|---|---|
| Sequence identifier | N | C | Start | End | Start | End | Start | End |
| ENST00000241124 | | | | | | | 1,337 | 1,452 |
| ENST00000356192 | | | 1 | 124 | 813 | 936 | | |
| ENST00000400065 | | | 15 | 124 | | | | |
| ENST00000400066 | | | 17 | 124 | 813 | 936 | | |
| GJB6 A | 7 | HT29 | | | | | | |
| GJB6 B | 4 | HT29 | 1 | 124 | 813 | 936 | | |
| GJB6 C | 2 | HT29 | 1 | 124 | | | | |
| GJB6 D | 2 | HT29 | 1 | 124 | 813 | 936 | | |
| GJB6 E | 1 | HT29 | | | 813 | 936 | | |
| GJB6 F | 1 | HT29 | | | | | | |

| | | | Exon 4 | | Exon 5 | | Exon 6 | | |
|---|---|---|---|---|---|---|---|---|---|
| Sequence identifier | N | C | Start | End | Start | End | Start | End | Start positions |
| ENST00000241124 | | | | | 2,569 | 2,738 | 8,823 | 10,355 | |
| ENST00000356192 | | | 1,511 | 1,620 | 2,569 | 2,738 | 8,823 | 10,355 | |
| ENST00000400065 | | | | | 2,569 | 2,738 | 8,823 | 10,347 | |
| ENST00000400066 | | | | | 2,569 | 2,738 | 8,823 | 10,347 | |
| GJB6 A | 7 | HT29 | | | | | 8,823 | 9,371 | 8,917, 8,917, 8,917, |
| | | | | | | | | | 9,122, 8,917, 8,916, |
| | | | | | | | | | 9,137 |
| GJB6 B | 4 | HT29 | | | 2,569 | 2,738 | 8,823 | 9,371 | 103, 112, 103, 110 |
| GJB6 C | 2 | HT29 | | | 2,569 | 2,738 | 8,823 | 9,371 | 103, 103 |
| GJB6 D | 2 | HT29 | | | 2,548 | 2,738 | 8,823 | 9,371 | 98, 98 |
| GJB6 E | 1 | HT29 | | | 2,569 | 2,738 | 8,823 | 9,371 | 861 |
| GJB6 F | 1 | HT29 | | | 2,524 | 2,738 | 8,823 | 9,371 | 2,524 |

Start/end sequence positions are indicated relative to start of exon 1 (ENSG00000121742; transcribed from minus strand; start position: 19,704,456 bp from chromosome 13 p-telomere; Ensembl release 50).

TABLE A-II-10
Exon positions from PRRX1.

| | | | Exon 1 | | Exon α | | Exon β | | Exon γ | |
|---|---|---|---|---|---|---|---|---|---|---|
| Sequence identifier | N | C | Start | End | Start | End | Start | End | Start | End |
| ENST00000239461 | | | 1 | 288 | | | | | | |
| ENST00000367760 | | | 1 | 288 | | | | | | |
| PRRX1 A | 7 | SW48 | | | | | | | | |
| PRXX1 B | 2 | SW48 | | | 50,433 | 50,663 | 51,315 | 51,367 | | |
| PRRX1 C | 2 | SW48 | | | 50,433 | 50,663 | | | | |
| PRRX1 D | 1 | SW48 | | | 50,433 | 50,663 | | | | |
| PRRX1 E | 1 | SW48 | | | 50,433 | 50,663 | | | | |
| PRRX1 F | 1 | SW48 | | | 50,433 | 50,663 | 51,315 | 51,367 | 53,778 | 53,840 |
| PRRX1 G | 1 | SW48 | | | | | | | 53,387 | 53,840 |
| PRRX1 H | 1 | SW48 | | | | | | | 53,387** | 53,840** |
| PRRX1 I | 1 | SW48 | | | | | | | | |

| | | | Exon δ | | Exon ε | | Exon 2 | | |
|---|---|---|---|---|---|---|---|---|---|
| Sequence identifier | N | C | Start | End | Start | End | Start | End | Start positions |
| ENST00000239461 | | | | | | | 55,555 | 55,731 | |
| ENST00000367760 | | | | | | | 55,555 | 55,731 | |
| PRRX1 A | 7 | SW48 | 53,969* | * | * | 54,761* | 55,555 | 55,663 | 54,492, 54,079, |
| | | | | | | | | | 54,627, 54,536, |
| | | | | | | | | | 54,495, 54,491, |
| | | | | | | | | | 54,356 |
| PRXX1 B | 2 | SW48 | 53,969 | 54,104 | 54,658 | 54,761 | 55,555 | 55,663 | 50,433, 50,433 |
| PRRX1 C | 2 | SW48 | 53,969* | * | * | 54,761* | 55,555 | 55,663 | 50,523, 50,507 |
| PRRX1 D | 1 | SW48 | 53,969 | 54,104 | | | 55,555 | 55,663 | 50,606 |
| PRRX1 E | 1 | SW48 | | | | | 55,555 | 55,663 | 50,494 |
| PRRX1 F | 1 | SW48 | 53,969 | 54,104 | | | 55,555 | 55,663 | 50,543 |
| PRRX1 G | 1 | SW48 | | | | | 55,555 | 55,663 | 53,450 |
| PRRX1 H | 1 | SW48 | 53,969 | 54,104 | 54,658 | 54,761 | 55,555 | 55,663 | 53,387 |
| PRRX1 I | 1 | SW48 | 53,969 | 54,104 | | | 55,555 | 55,663 | 54,037 |

Start/end sequence positions are indicated relative to start of exon 1 (ENSG00000116132; transcribed from plus strand; start position: 168,899,937 bp from chromosome 1 p-telomere; Ensembl release 50).
*Exon δ in sequence PRXX1 B and PRRX1 C is a retention of the intron between exons δ and ε.
**The exon lacks bases 53,625-53,778

TABLE A-II-11
Exon positions from PRRX2.

| | | | Exon 1 | | Exon 2 | | Exon 3 | | Exon 4 | | Start |
|---|---|---|---|---|---|---|---|---|---|---|---|
| Sequence identifier | N | T | Start | End | Start | End | Start | End | Start | End | positions |
| ENST00000372469 | | | 1 | 486 | 53,591 | 53,779 | 54,956 | 55,135 | 56,577 | 57,031 | |
| PRRX2 A | 11 | C1033III | | | | | 55,074 | 55,135 | 56,577 | 56,922 | 55,074 for all |
| | | | | | | | | | | | 11 clones |
| PRRX2 B | 1 | C1033III | | | | | | | 56,689 | 56,922 | 56,689 |

Start/end sequence positions are indicated relative to start of exon 1 (ENSG00000167157; transcribed from plus strand; start position: 131,467,741 bp from chromosome 9 p-telomere; Ensembl release 50).

1. A method for the detection of abnormal gene expression of at least one gene, wherein said at least one gene is VNN1, said method comprising:
a) identifying an expression level of at least one RNA transcript variant of said at least one gene in a sample obtained from a test subject,
b) comparing the expression level of said at least one RNA transcript variant of said at least one gene with a reference obtained from a reference subject,
c) selecting a desired sensitivity,
d) selecting a desired specificity,
e) classifying the test subject as one likely to have an abnormal gene expression, if the expression level of said at least one RNA transcript variant of said at least one gene in the sample obtained from the test subject is different from the reference, and classifying the test subject as one unlikely to have an abnormal gene expression, if the expression level of said at least one RNA transcript variant of said at least one gene is equal to the reference.

2-12. (canceled)

13. A method for the detection of abnormal gene expression of at least one gene, wherein said at least one gene is VNN1, said method comprising determining an expression level of at least one RNA transcript variant of said at least one gene in a sample obtained from a test subject.

14. The method according to claim 13 further comprising:
b) comparing the expression level of said at least one RNA transcript variant of said at least one gene with a reference obtained from a reference subject,
c) selecting a desired sensitivity,
d) selecting a desired specificity,
e) classifying the test subject as one likely to have an abnormal gene expression, if the expression level of said at least one RNA transcript variant of said at least one gene in the sample obtained from a test subject is different from the reference, and classifying the test subject as one unlikely to have an abnormal gene expression, if the expression level of said at least one RNA transcript variant of said at least one gene is equal to the reference.

15. The method according to claim 1, wherein the expression level of said at least one RNA transcript variant of said gene in the test subject is higher than the reference subject.

16. The method according to claim 1, wherein said sample is selected from the group consisting of blood, serum, plasma, faeces, tissue biopsy, and culture cells.

17. The method according to claim 1, wherein the abnormal expression pattern is indicative of the presence of cancer, a precursor to cancer, an inflammatory disease, a viral infection or a metabolic disease in the test subject.

18. The method according to claim 17, wherein the cancer is colorectal cancer or the precursor to cancer is colorectal adenomas.

19. The method according to claim 1, wherein the VNN1 RNA transcript variant is selected from the group consisting of VNN1 A (SEQ ID NO:15), VNN1 B (SEQ ID NO: 16), and VNN1 C (SEQ ID NO: 17).

20. The method according to claim 1, wherein the VNN1 RNA transcript variant comprises one or more of the exons selected from the group consisting of VNN1α (SEQ ID NO:131), VNN1α′ (SEQ ID NO:132), VNN1α″ (SEQ ID NO133), VNN1β (SEQ ID NO:134), and VNN1β′ (SEQ ID NO:135).

21. A method of using an RNA transcript variant for diagnosing, prognosing, or monitoring the progression of a disease in a subject comprising:
identifying the amount of an RNA transcript variant in a sample from said subject, wherein said RNA transcript variant is selected from the group consisting of (SEQ ID NO:15), (SEQ ID NO:16), (SEQ ID NO:17), (SEQ ID NO:18), (SEQ ID NO:131), (SEQ ID NO:132), (SEQ ID NO:133), (SEQ ID NO:134), and (SEQ ID NO:135); and comparing the amount of the RNA transcript variant in the sample to a reference level for said RNA transcript variant.

22. The method according to claim 21, wherein said disease is cancer.

23. The method according to claim 22, wherein said cancer is colorectal cancer.
